(19) United States
(12) Patent Application Publication
(10) Pub. No.: US 2004/0180048 A1
(43) Pub. Date: Sep. 16, 2004

(54) Neuronal and retinal gene expression patterns

(75) Inventors: Donald Jeffery Zack, Baltimore, MD (US); Abigail Shoshanna Hackam, Baltimore, MD (US)

Correspondence Address: BANNER & WITCOFF, 1001 G STREET N W, SUITE 1100, WASHINGTON, DC, 20001, US

(73) Assignee: The Johns Hopkins University, Baltimore, MD

(21) Appl. No.: 10/618,084

(22) Filed: Jul. 14, 2003

Related U.S. Application Data
(60) Provisional application No. 60/395,460, filed on Jul. 12, 2002.

(51) Int. Cl. A61K 39/395; C12N 5/08

(52) U.S. Cl. 424/143.1; 435/368

(57) ABSTRACT

The retinal degeneration (rd1) mutant mouse exhibits rapid rod photoreceptor degeneration caused by a mutation in the rod photoreceptor-specific gene cGMP phosphodiesterase β (PDE). One intriguing aspect of the rd1 phenotype is a secondary wave of cone photoreceptor death that follows loss of rods. In this study, we investigated gene expression changes associated with the progression of photoreceptor degeneration in rd1 mice using a custom retina microarray. The microarray contains 5,376 DNA fragments that correspond to mouse genes known or postulated to be involved in normal retinal function, development, or disease. Gene expression in rd1 retina was compared with age-matched wild-type controls at three time-points corresponding to critical stages in retina degeneration: peak of rod degeneration, early in cone degeneration and during cone degeneration. Statistical significance analyses demonstrated that approximately 3% of the genes on the microarray were differentially expressed, including known genes and genes that had not been previously implicated in degeneration. Interestingly, there was less overlap in the genes that were upregulated at each stage of degeneration, suggesting the involvement of distinct molecular pathways. Genes involved in transport, signalling and cytoskeleton were differentially expressed during rod degeneration whereas genes involved in growth and proliferation, oxidative stress and protein modification were increased prior to and during cone degeneration. These results provide clues to underlying molecular processes occurring during photoreceptor degeneration, and provide direction for future cell-based studies.

This application claims the benefit of provisional application serial No. 60/395,460 filed Jul. 12, 2002, the disclosure of which is expressly incorporated herein.

### TECHNICAL FIELD OF THE INVENTION

This invention is related to the area of neuronal cell death. In particular, it relates to genes which are characteristically dyregulated in neuronal cells, including retinal cells, which are subjected to a lethal challenge.

### BACKGROUND OF THE INVENTION

The molecular events contributing to retinal disease are highly complex, reflecting the interdependence among the different cell types in the retina and the intimate cellular contacts between photoreceptors and the surrounding epithelium. Mutations in over 80 genes have been implicated in retinal degenerations and over 50 additional disease loci have been mapped (RetNet, URL address: www host server, sph.uth.tmc.edu domain name, Retnet/home.htm directory). Many of the disease-causing genes have been identified as key players in retina function, including enzymes involved in phototransduction and the visual cycle, transcription factors that control retina-specific gene expression, and structural proteins that support the unique disk membrane structure of photoreceptors (TINS review). Despite knowledge of the functions of many of these proteins, the cellular and molecular pathways leading from the primary mutation to photoreceptor death are not well understood.

Naturally occurring and experimentally generated rodent models have been valuable for exploring the functional and histopathological changes that occur in retina disease {Chang 2002; Br J Ophth review}. One well-studied model is the retinal degeneration (rd1) mutant mouse, which has a deficiency in the rod photoreceptor-specific cGMP phosphodiesterase β-subunit (PDE). The phenotype of the rd1 mutant illustrates several questions applicable to retina diseases in general. First, although early changes in cGMP and calcium dynamics have been described in homozygous rd1/rd1 mice {Lolley 1977; Faber 1994; Farber 1995}, the molecular events that result in photoreceptor apoptosis are unclear. Secondly, a secondary wave of degeneration of the less prevalent cone photoreceptors occurs after the loss of rods. The basis for this non-autonomous cone degeneration, which is also observed in humans, has been hypothesized to involve diffusible or contact-mediated factors from neighboring cells {Huang 1993; Sahel ref}. However, the molecular events and underlying gene expression changes contributing to this phenomenon are not known. Thirdly, abundant morphological and molecular changes have been measured in other retinal cells during and after photoreceptor degeneration (Italian, Boston refs), but the mechanisms behind these changes, and role of these cells in promoting or protecting photoreceptors from apoptosis, is not understood.

The coordinated expression of multiple genes is expected to contribute to rod and cone photoreceptor death and to the reactive changes occurring in other retina cells. The recent completion of genome sequencing efforts and the accumulation of tissue libraries and EST databases has resulted in dramatic advances in the ability to rapidly characterize gene expression. A number of recent studies have used various gene profiling methodologies to identify novel retina genes and to investigate the pathogenesis of retina disease {Swaroop and Zack, 2002; Wilson 2002}. These methods differ in their application. For example, two techniques, data mining of cDNA libraries {Sinha 2000; Wistow, G. Bernstein, S. L., 2002; Stoehr 2002; Katsanis 2002} and SAGE (serial analysis of gene expression) {Blackshaw et al. 2001 }, are highly suited for quantitation of gene expression under a single experimental state. In comparison, microarray technologies have the advantage of being able to measure gene expression changes across multiple experimental conditions or different disease states. Several types of microarrays, including oligonucleotide and cDNA, custom and commercial, glass and membrane, have been used in investigations of retina disease {Faijo 2002; Livesay 2000; Kennan 2002; Jun 2001; Joussen 2001; Buraczynska 2002}. Important insights into pathogenic pathways have been gained from such studies, and in at least one example, a new retina disease-causing gene was identified using microarray analysis {Kennan, 2002}.

One aspect arising from the microarray analyses above has been the recognition that custom cDNA and commercial oligonucleotide microarrays have different strengths. Although commercial arrays typically have an order of magnitude more genes, custom arrays may be more appropriate for some research questions. A critical component of microarrays is their dependence, and therefore potential limitation, on the identity of the genes on the array. Many commercially available arrays often do not have a large representation of genes expressed in a tissue as highly specialized as the retina. Low representation of genes from the retina could curtail the discovery of novel retina genes and hinder exploration of disease mechanisms. Secondly, the considerable cost of commercial arrays limits the number of experiments that can be performed. However, once established, custom arrays can be generated at a reasonable cost, increasing the number of replicates and time-points, and consequently improving the statistical analyses.

Finally, recently published calibration experiments demonstrated that fold-change measurements using custom cDNA arrays were more predictable and accurate, and had less uncontrolled bias than oligonucleotide arrays {Yuen, 2002}.

There is a continuing need in the art to characterize degenerating or dying neuronal cells relative to normal neuronal cells so that any differences can be exploited for therapeutic and diagnostic benefits.

### SUMMARY OF THE INVENTION

A first embodiment of the invention provides a method for inhibiting neuronal cell death in a mammalian subject. An effective amount of an isolated molecule comprising an antibody variable region is administered to a subject in need thereof. The antibody variable region specifically binds to a neuronal marker (NM) protein selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone R11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family. A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KLAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6. Neuronal cell death is thereby inhibited.

A second embodiment of the invention provides a method for preventing neuronal cell death in a mammal. A nucleic acid molecule comprising a coding sequence for a NM protein is administed to the mammal. The coding sequence is selected from the group consisting of: NM Mus musculus retinal S antigen; Mus musculus neural retina leucine zipper gene; M musculus photoreceptor specific protein PSP G145; IMAGE 4507893 5; Mus musculus domesticus phosducin; IMAGE 4507284 5; Danio rerio brain type fatty acid binding protein; M musculus X linked juvenile retinoschisis protein; M musculus guanine nucleotide binding protein beta 1 Gnb1; Mus musculus TPA regulated locus; Mouse nuclear protein mdm 1; IMAGE 4511806 5; M musculus male germ cell associated kinase; heat shock protein 60 kDaMm 1777; no match17; NCI CGAP BC3 Mus musculus cDNA clone IMAGE 3976794; no homo16; Homo sapiens CGI 45 protein; ESTsMm 44103; Mouse opsin MOPS; IMAGE 4225062 5; Mm 100212; H sapiens fer fps fes related tyrosine kinase phosphoprotein NCP94 FER; IMAGE 4505626 5 602393946F1 NIH MGC 94; solute carrier family 12 member 2Mm 4168; Mus musculus BUB2 like protein 1 HBLP1 mRNA complete cdsMm 104771; hemoglobin Y homeodomain protein crx; promininMm 6250; no homo13; IMAGE 1279184 5; Human microfibril associated glycoprotein 4; Mm 70462; no match A; Rattus sp mRNA for BHF 1; ribosomal protein S24Mm 16775; Stratagene mouse Tcell 937311 IMAGE 1002041; NCI CGAP Kid14 Mus IMAGE 4236354 5; R norvegicus retinoblastoma binding protein 9; Mus musculus exostoses multiple 1 Ext1; selectin endothelial cell ligandMm 488; ESTs Weakly similar to HYPOTHETICAL 16 1 KD PROTEIN IN SEC17 QCR1 INTERGENIC REGION Saccharomyces cerevisiae Mm 27114; ESTs Highly similar to KIAA0824 protein H sapiens Mm 34579; Mus musculus ribospsmal protein L10A Rp110a; R norvegicus ribonucleoprotein F; clone 1110007F23; no match38; M musculus Srp20 gene; homeodomain interacting protein kinase 2Mm 20934; FSHD region gene 1Mm 67; UI M BH3 ari c 10 0 UI s1 NIH BMAP M S4; Homo sapiens CED 6 protein CED 6; Mus musculus RIKEN clone 0610009E22; RAB18 member RAS oncogene familyMm 22660; no match5; Mus musculus prominin Prom; ribosomal protein L12Mm 70127; ESTs Highly similar to ELONGATION FACTOR 1 DELTA Homo sapiens Mm 21086; ESTs Highly similar to HYPOTHETICAL 37 2 KD PROTEIN C12C2 09C IN CHROMOSOME I Schizosaccharomyces pombe Mm 21383; clone 3021401C12; M musculus very long chain acyl CoA dehydrogenase; vitronectinMm 3667; ESTs Weakly similar to LIV 1 protein H sapiens Mm 41214; Mus musculus dopamine receptor 4; no match7; ATPase H transporting lysosomal vacuolar proton pump noncatalytic accessory protein 1 110 160 kDa Mm 20869; Rattus norvegicus partial mRNA for CRM1 protein; eukaryotic translation elongation factor 1 alpha 1Mm 16317; Human karyopherin beta2 importin; ESTs Moderately similar to hypothetical protein H sapiens Mm 22878; Homo sapiens PAC clone RP4 687K1; UI M AO1 aeh e 11 0 UI r1 NIH BMAP MPG N; high mobility group protein 14Mm 2756; ESTsMm 31374; R norvegicus aryl hydrocarbon interacting protein like 1; UI M CG0p bmu h 08 0 UI s1 NIH BMAP Ret4 S2; RAB10 member RAS oncogene familyMm 9455; Mus musculus early development regulator 2; no match83; Mus musculus topoisomerase DNA II beta; alpha tubulin; Homo sapiens MTA1 L1; retinitis pigmentosa GTPase regulator interacting protein 1 Mm 21662; Mus musculus FXYD dom containing ion transport regulator 5; Mus musculus cytochrome P450 3A25 CYP3A25 mRNA complete cdsMm 26993; IMAGE 4505626 5; RNA polymerase II transcriptional coactivatorMm 966; ESTs Highly similar to CAAX prenyl protease H sapiens Mm 34399; Soares mammary gland NbMMG IMAGE 1347586; clone 2700067D09; ESTs Weakly similar to defline not available 5901802 D melanogaster Mm 35127; torsin family 1 member AMm 29151; Mm 23086; M musculus brain cyclic nucleotide gated K; Mus musculus N myc downstream regulated 1; Homo sapiens splicing factor 3b subunit 3; Mus musculus mRNA for Lim homeodomain protein Islet1Mm 42242; Mouse mRNA for syntaxin 3D 1; Mus musculus chromosome 7 clone 19K5; ES18 proteinMm 23296; ESTs Highly similar to KIAA0729 protein H sapiens Mm 13148; ESTsMm 33949; Rat transcription factor RZR beta gene; ESTs Moderately similar to hypothetical protein H sapiens Mm 30235; Homo sapiens KLAA0009 gene product; no match X; ESTs Moderately similar to MYOSIN LIGHT CHAIN KINASE Dictyostelium discoideum Mm 1881; serum glucocorticoid regulated kinaseMm 28405; ESTs Weakly similar to cappuccino D melanogaster Mm 41762; regulator of G protein signaling 9Mm 38548; ESTsMm 34351; ESTsMm 32460; Mm 44404; ESTsMm 37515; Mus musculus cytochrome P450 2f2 Cyp2f2; Finkel Biskis Reilly murine sarcoma virus FBR MuSV ubiquitously expressed fox derived Mm 4890; guanylate cyclase activator la retina Mm 16224; human CRX control; adducin 2 beta Mm 104155; mouse CRX control; NRL control; Mus musculus ELOVL4; Mus musculus N myc downstream regulated 3; lactate dehydrogenase 1 A chainMm 26504; ESTs Moderately similar to stromelysin PDGF responsive element binding protein transcription factor M musculus Mm 38372; ESTsMm 11285; M musculus chr 10 clone RP21 39C4; ESTs Highly similar to 40 KD PEPTIDYL PROLYL CIS TRANS ISOMERASE Homo sapiens Mm 30242; NIH BMAP Ret4 S2 Mus UI M CG0p big e 08 0 UI 3; Soares mammary gland NMLMG IMAGE 3467149; glycosylphosphatidylinositol 1 homolog human Mm 6354; Rattus norvegicus NMDA receptor subunit NR2; ESTsMm 33788; Mus musculus hexokinase 1 Hk1; inosine 5 phosphate dehydrogenase 2Mm 6065; N myc downstream regulated 3Mm 36775; no match V; villin 2Mm 4551; Rattus norvegicus TM6P1 TM6P1; Mus musculus mRNA for heterogeneous nuclear ribonucleoprotein HMm 21740; ESTsMm 103333; Mus musculus retinal taurine transporter; Mus musculus poly rC binding protein; ESTs Weakly similar to nuclear poly C binding protein M musculus Mm 29707; ESTs Weakly similar to similar to 1 acyl glycerol 3 phosphate acyltransferases C elegans Mm 24117; Mm 27013; pre B cell leukemia transcription factor 3Mm 7331; ESTsMm 21299; Mus musculus kinectin 1; Mus musculus drebrin A mRNA complete cdsMm 104044; H3087H01 5 NIA Mouse 15K cDNA Clone Set; SAC483 Mouse e14 5 developing pituitary gland; cloneE130113K08; Mus musculus major histocompatibility locus class II region Fas binding protein Daxx DAXX gene partial cds Bing1 BING1 tapasin tapasin RalGDS like factor RLF KE2 KE2 BING4 BING4 beta1 3 galactosyl transferase beta1 3 galactosylMm 20926; Mus musculus aquaporin 1; acyl Coenzyme A dehydrogenase very long chainMm 18630; Mouse proprotein convertase 4; M musculus activating transcription factor 4 Atf4; guanine nucleotide binding protein beta 5Mm 4702; phosducin control; ESTsMm 38578; Barstead bowel MPLRB9 IMAGE 1095982; M musculus stromal cell derived factor recep; ESTs Weakly similar to E04F6 2 gene product C elegans Mm 18889; IMAGE 963149 5; syntaxin binding protein 1 Mm 3129; solute carrier family 16 monocarboxylic acid transporters member 1Mm 9086; ESTs Highly similar to TRICARBOXYLATE TRANSPORT PROTEIN PRECURSOR Rattus norvegicus Mm 22679; Bcl2 likeMm 3882; Soares mouse p3NMF19 5 IMAGE 493296; Mus musculus beta galactosidase complex; H sapiens ADP ribosylation factor binding protein GGA2; Mm 31266; IMAGE 560050 5; Mus musculus DXHXS6673E protein DXHXS6673E mRNA complete cdsMm 23458; M musculus mRNA for hair keratin mHb6; Mus musculus thyroglobulin; ESTs Moderately similar to KIAA0956 protein H sapiens Mm 11428; H3050H05 3 NIA Mouse 15K cDNA Clone Set; ESTs Moderately similar to signal recognition particle 54K protein M musculus Mm 32508; Mouse PSD 95 SAP90A; ESTsMm 29308; alkaline phosphatase 2 liverMm 1265; Homo sapiens 12 seeders BAC RP11 19E18; ESTsMm 41269; ESTsMm 86724; Homo sapiens 12q13 1 PAC RPCI1 228P16; serine threonine kinase receptor associated proteinMm 22584; UI M BZ0 axl a 11 0 UI s1 NIH BMAP MHI2; Mus musculus poly rC binding protein 2; IMAGE 4503171 5; ESTsMm 35430; activating transcription factor 4Mm 641; Mouse serine threonine phosphatase 2C; GAPDH control; Human mRNA for KIAA0299; ESTs Weakly similar to proline rich protein M musculus Mm 41665; megakaryocyte associated tyrosine kinaseMm 2918; homer neuronal immediate early gene 2Mm 228; peroxisomal farnesylated proteinMm 29198; blank; zinc finger protein 238Mm 27962; ESTs Highly similar to PHENYLALANYL TRNA SYNTHETASE BETA CHAIN CYTOPLASMIC Saccharomyces cerevisiae Mm 27403; Rat microtubule associated protein 2 MAP2; timeless homolog Drosophila Mm 6458; kinectin 1Mm 3110; phosphatidylinositol membrane associatedMm 1860; R norvegicus CDP diacylglycerol synthase; Homo sapiens DKFZp434A132; Mus musculus hematopoietic zinc finger; mitogen activated protein kinase kinase 7Mm 3906; H3110G03 3 NIA Mouse 15K cDNA; ESTs Highly similar to HYPOTHETICAL 47 9 KD PROTEIN B0303 3 IN CHROMOSOME III Caenorhabditis elegans Mm 30147; ESTs Highly similar to CELL GROWTH REGULATING NUCLEOLAR PROTEIN M musculus Mm 28560; no match W; Mouse endogenous murine leukemia virus polytropic provirus DNA; clone1110013A05; aryl hydrocarbon receptorMm 4452; peroxisome proliferator activated receptor alphaMm 1373; Mus musculus LAG protein Lag Rattus NMDA receptor glutamate binding subunit; Mus musculus syntaxin binding protein 1; Mus musculus MAP kinase phosphatase 6; Rattus norvegicus retina specific protein PAL; no match33; Mus musculus myc box dependent interacting pro; Murine leukemia virus erv1 envelope protein; cytochrome c oxidase subunit VIIa 3Mm 2151; proteasome prosome macropain subunit alpha type 3Mm 1007; Homo sapiens mRNA cDNA DKFZp434N1615; Mus musculus TCR beta locus; ESTs Weakly similar to LOK M musculus Mm 74661; small inducible cytokine subfamily A member 22Mm 12895; ESTsMm 23682; no match I; no match H; high mobility group protein I isoform CMm 3953; protein kinase cAMP dependent catalytic alphaMm 22479; Mus musculus phosphatidylinositol membrane associated; no match G; Mouse heparin binding epidermal growth factor like; Homo sapiens cDNA DKFZp586B0924; Mouse magnesium dependent protein; ESTs Weakly similar to ZW10 interactor Zwint H sapiens Mm 38994; ESTsMm 30480; H sapiens ADP ribosylation factor GTPase activating protein 1; Mus elongation of very long chain fatty acids; Mouse Y box binding protein 1 DNA binding MSY 1; Homo sapiens KIAA0249 gene product; Mus musculus Ran binding protein 2; Mus musculus histidine decarboxylase cluster; Homo sapiens cDNA FLJ21612 fis clone COL07355; UI M BH2 3 aqc g 10 0 UI 5; Rattus norvegicus APP binding protein 1; Mus musculus beta site APP cleaving enzyme; DNA methyltransferase cytosine 5 Mm 7814; no match66; ESTs Weakly similar to Lpi2p S cerevisiae Mm 21859; R norvegicus phosphatidylinositol synthase; ribonuclease L 2 5 oligoisoadenylate synthetase dependent inhibitorMm 5831; Mm 104074; H sapiens protein phosphatase 2A regulatory subunit B; H3147A11 5 NIA Mouse 15K cDNA Clone Set; Mus musculus Y box transcription factor; Mouse gene for basigin; Homo sapiens mRNA for FLJ00042 protein; R norvegicus nup155 nucleoporin 155kD; tubby like protein 1 Mm 42102; R norvegicus RNA binding protein SiahBP; UI M BZ0 axj h 06 0 UI 3; and Mus musculus pyruvate kinase 3. Neuronal cell death in the mammal is thereby inhibited or prevented.

A third embodiment of the invention is a method for preventing neuronal cell death in a mammal. A purified human NM protein selected from the group consisting of: NM Mus musculus retinal S antigen; Mus musculus neural retina leucine zipper gene; M musculus photoreceptor specific protein PSP G145; IMAGE 4507893 5; Mus musculus domesticus phosducin; IMAGE 4507284 5; Danio rerio brain type fatty acid binding protein; M musculus X linked juvenile retinoschisis protein; M musculus guanine nucleotide binding protein beta 1 Gnb1; Mus musculus TPA regulated locus; Mouse nuclear protein mdm 1; IMAGE 4511806 5; M musculus male germ cell associated kinase; heat shock protein 60 kDaMm 1777; no match17; NCI CGAP BC3 Mus musculus cDNA clone IMAGE 3976794; no homo16; Homo sapiens CGI 45 protein; ESTsMm 44103; Mouse opsin MOPS; IMAGE 4225062 5; Mm 100212; H sapiens fer fps fes related tyrosine kinase phosphoprotein NCP94 FER; IMAGE 4505626 5 602393946F1 NIH MGC 94; solute carrier family 12 member 2Mm 4168; Mus musculus BUB2 like protein 1 HBLP1 mRNA complete cdsMm 104771; hemoglobin Y beta like embryonic chainMm 35830; erythrocyte protein band 4 1Mm 30038; no match55; Mus musculus MYLE protein mRNA complete cdsMm 41091; RIKEN full length enriched adult male hypothalamus musculus cDNA clone A230050E13; NCI CGAP Mam6 Mus IMAGE 3500058; Mus musculus mRNA for GTP binding protein drg2 gene Mm 41803; Homo sapiens mRNA for KIAA1549 protein; Mus musculus karyopherin importin alpha 2 Kpna2; UI M BZ1 bkv b 01 0 UI 3; no match B; ESTsMm 939; Mus musculus cDNA sequence AF244542; IMAGE 1348390 5; solute carrier family 30 zinc transporter member 3Mm 1396; no match110; Mus musculus homeodomain protein crx; promininMm 6250; no homo13; IMAGE 1279184 5; Human microfibril associated glycoprotein 4; Mm 70462; no match A; Rattus sp mRNA for BHF 1; ribosomal protein S24Mm 16775; Stratagene mouse Tcell 937311 IMAGE 1002041; NCI CGAP Kid14 Mus IMAGE 4236354 5; R norvegicus retinoblastoma binding protein 9; Mus musculus exostoses multiple 1 Ext1; selectin endothelial cell ligandMm 488; ESTs Weakly similar to HYPOTHETICAL 16 1 KD PROTEIN IN SEC17 QCR1 INTERGENIC REGION Saccharomyces cerevisiae Mm 27114; ESTs Highly similar to KIAA0824 protein H sapiens Mm 34579; Mus musculus ribospsmal protein L10A Rp110a; R norvegicus ribonucleoprotein F; clone 11 10007F23; no match38; M musculus Srp20 gene; homeodomain interacting protein kinase 2Mm 20934; FSHD region gene 1Mm 67; UI M BH3 ari c 10 0 UI s1 NIH BMAP M S4; Homo sapiens CED 6 protein CED 6; Mus musculus RIKEN clone 0610009E22; RAB18 member RAS oncogene familyMm 22660; no match5; Mus musculus prominin Prom; ribosomal protein L12Mm 70127; ESTs Highly similar to ELONGATION FACTOR 1 DELTA Homo sapiens Mm 21086; ESTs Highly similar to HYPOTHETICAL 37 2 KD PROTEIN C12C2 09C IN CHROMOSOME I Schizosaccharomyces pombe Mm 21383; clone 3021401C12; M musculus very long chain acyl CoA dehydrogenase; vitronectinMm 3667; ESTs Weakly similar to LIV 1 protein H sapiens Mm 41214; Mus musculus dopamine receptor 4; no match7; ATPase H transporting lysosomal vacuolar proton pump noncatalytic accessory protein 1 110 160 kDa Mm 20869; Rattus norvegicus partial mRNA for CRM1 protein; eukaryotic translation elongation factor 1 alpha 1Mm 16317; Human karyopherin beta2 importin; ESTs Moderately similar to hypothetical protein H sapiens Mm 22878; Homo sapiens PAC clone RP4 687K1; UI M AO1 aeh e 11 0 UI r1 NIH BMAP MPG N; high mobility group protein 14Mm 2756; ESTsMm 31374; R norvegicus aryl hydrocarbon interacting protein like 1; UI M CG0p bmu h 08 0 UI s1 NIH BMAP Ret4 S2; RAB10 member RAS oncogene familyMm 9455; Mus musculus early development regulator 2; no match83; Mus musculus topoisomerase DNA II beta; alpha tubulin; Homo sapiens MTA1 L1; retinitis pigmentosa GTPase regulator interacting protein 1 Mm 21662; Mus musculus FXYD dom containing ion transport regulator 5; Mus musculus cytochrome P450 3A25 CYP3A25 mRNA complete cdsMm 26993; IMAGE 4505626 5; RNA polymerase II transcriptional coactivatorMm 966; ESTs Highly similar to CAAX prenyl protease H sapiens Mm 34399; Soares mammary gland NbMMG IMAGE 1347586; clone 2700067D09; ESTs Weakly similar to defline not available 5901802 D melanogaster Mm 35127; torsin family 1 member AMm 29151; Mm 23086; M musculus brain cyclic nucleotide gated K; Mus musculus N myc downstream regulated 1; Homo sapiens splicing factor 3b subunit 3; Mus musculus mRNA for Lim homeodomain protein Islet1Mm 42242; Mouse mRNA for syntaxin 3D 1; Mus musculus chromosome 7 clone 19K5; ES18 proteinMm 23296; ESTs Highly similar to KIAA0729 protein H sapiens Mm 13148; ESTsMm 33949; Rat transcription factor RZR beta gene; ESTs Moderately similar to hypothetical protein H sapiens Mm 30235; Homo sapiens KIAA0009 gene product; no match X; ESTs Moderately similar to MYOSIN LIGHT CHAIN KINASE Dictyostelium discoideum Mm 1881; serum glucocorticoid regulated kinaseMm 28405; ESTs Weakly similar to cappuccino D melanogaster Mm 41762; regulator of G protein signaling 9Mm 38548; ESTsMm 34351; ESTsMm 32460; Mm 44404; ESTsMm 37515; Mus musculus cytochrome P450 2f2 Cyp2f2; Finkel Biskis Reilly murine sarcoma virus FBR MuSV ubiquitously expressed fox derived Mm 4890; guanylate cyclase activator 1a retina Mm 16224; human CRX control; adducin 2 beta Mm 104155; mouse CRX control; NRL control; Mus musculus ELOVL4; Mus musculus N myc downstream regulated 3; lactate dehydrogenase 1 A chainMm 26504; ESTs Moderately similar to stromelysin PDGF responsive element binding protein transcription factor M musculus Mm 38372; ESTsMm 11285; M musculus chr 10 clone RP21 39C4; ESTs Highly similar to 40 KD PEPTIDYL PROLYL CIS TRANS ISOMERASE Homo sapiens Mm 30242; NIH BMAP Ret4 S2 Mus UI M CG0p big e 08 0 UI 3; Soares mammary gland NMLMG IMAGE 3467149; glycosylphosphatidylinositol 1 homolog human Mm 6354; Rattus norvegicus NMDA receptor subunit NR2; ESTsMm 33788; Mus musculus hexokinase 1 Hk1; inosine 5 phosphate dehydrogenase 2Mm 6065; N myc downstream regulated 3Mm 36775; no match V; villin 2Mm 4551; Rattus norvegicus TM6P1 TM6P1; Mus musculus mRNA for heterogeneous nuclear ribonucleoprotein HMm 21740; ESTsMm 103333; Mus musculus retinal taurine transporter; Mus musculus poly rC binding protein; ESTs Weakly similar to nuclear poly C binding protein M musculus Mm 29707; ESTs Weakly similar to similar to 1 acyl glycerol 3 phosphate acyltransferases C elegans Mm 24117; Mm 27013; pre B cell leukemia transcription factor 3Mm 7331; ESTsMm 21299; Mus musculus kinectin 1; Mus musculus drebrin A mRNA complete cdsMm 104044; H3087H01 5 NIA Mouse 15K cDNA Clone Set; SAC483 Mouse e14 5 developing pituitary gland; cloneE130113K08; Mus musculus major histocompatibility locus class II region Fas binding protein Daxx DAXX gene partial cds Bing1 BING1 tapasin tapasin RalGDS like factor RLF KE2 KE2 BING4 BING4 beta1 3 galactosyl transferase beta1 3 galactosylMm 20926; Mus musculus aquaporin 1; acyl Coenzyme A dehydrogenase very long chainMm 18630; Mouse proprotein convertase 4; M musculus activating transcription factor 4 Atf4; guanine nucleotide binding protein beta 5Mm 4702; phosducin control; ESTsMm 38578; Barstead bowel MPLRB9 IMAGE 1095982; M musculus stromal cell derived factor recep; ESTs Weakly similar to E04F6 2 gene product C elegans Mm 18889; IMAGE 963149 5; syntaxin binding protein 1 Mm 3129; solute carrier family 16 monocarboxylic acid transporters member 1Mm 9086; ESTs Highly similar to TRICARBOXYLATE TRANSPORT PROTEIN PRECURSOR Rattus norvegicus Mm 22679; Bcl2 likeMm 3882; Soares mouse p3NMF19 5 IMAGE 493296; Mus musculus beta galactosidase complex; H sapiens ADP ribosylation factor binding protein GGA2; Mm 31266; IMAGE 560050 5; Mus musculus DXHXS6673E protein DXHXS6673E mRNA complete cdsMm 23458; M musculus mRNA for hair keratin mHb6; Mus musculus thyroglobulin; ESTs Moderately similar to KIAA0956 protein H sapiens Mm 11428; H3050H05 3 NIA Mouse 15K cDNA Clone Set; ESTs Moderately similar to signal recognition particle 54K protein M musculus Mm 32508; Mouse PSD 95 SAP90A; ESTsMm 29308; alkaline phosphatase 2 liverMm 1265; Homo sapiens 12 seeders BAC RP11 19E18; ESTsMm 41269; ESTsMm 86724; Homo sapiens 12q13 1 PAC RPCI1 228P16; serine threonine kinase receptor associated proteinMm 22584, UI M BZ0 axl a 11 0 UI s1 NIH BMAP MHI2; Mus musculus poly rC binding protein 2; IMAGE 4503171 5; ESTsMm 35430; activating transcription factor 4Mm 641; Mouse serine threonine phosphatase 2C; GAPDH control; Human mRNA for KIAA0299; ESTs Weakly similar to proline rich protein M musculus Mm 41665; megakaryocyte associated tyrosine kinaseMm 2918; homer neuronal immediate early gene 2Mm 228; peroxisomal farnesylated proteinMm 29198; blank; zinc finger protein 238Mm 27962; ESTs Highly similar to PHENYLALANYL TRNA SYNTHETASE BETA CHAIN CYTOPLASMIC Saccharomyces cerevisiae Mm 27403; Rat microtubule associated protein 2 MAP2; timeless homolog Drosophila Mm 6458; kinectin 1Mm 3110; phosphatidylinositol membrane associatedMm 1860; R norvegicus CDP diacylglycerol synthase; Homo sapiens DKFZp434A132; Mus musculus hematopoietic zinc finger; mitogen activated protein kinase kinase 7Mm 3906; H3110G03 3 NIA Mouse 15K cDNA; ESTs Highly similar to HYPOTHETICAL 47 9 KD PROTEIN B0303 3 IN CHROMOSOME III Caenorhabditis elegans Mm 30147; ESTs Highly similar to CELL GROWTH REGULATING NUCLEOLAR PROTEIN M musculus Mm 28560, no match W; Mouse endogenous murine leukemia virus polytropic provirus DNA; clone1110013A05; aryl hydrocarbon receptorMm 4452; peroxisome proliferator activated receptor alphaMm 1373; Mus musculus LAG protein Lag Rattus NMDA receptor glutamate binding subunit; Mus musculus syntaxin binding protein 1; Mus musculus MAP kinase phosphatase 6; Rattus norvegicus retina specific protein PAL; no match33; Mus musculus myc box dependent interacting pro; Murine leukemia virus erv1 envelope protein; cytochrome c oxidase subunit VIIa 3Mm 2151; proteasome prosome macropain subunit alpha type 3Mm 1007; Homo sapiens mRNA cDNA DKFZp434N1615; Mus musculus TCR beta locus; ESTs Weakly similar to LOK M musculus Mm 74661; small inducible cytokine subfamily A member 22Mm 12895; ESTsMm 23682; no match I; no match H; high mobility group protein I isoform CMm 3953; protein kinase cAMP dependent catalytic alphaMm 22479; Mus musculus phosphatidylinositol membrane associated; no match G; Mouse heparin binding epidermal growth factor like; Homo sapiens cDNA DKFZp586B0924; Mouse magnesium dependent protein; ESTs Weakly similar to ZW10 interactor Zwint H sapiens Mm 38994; ESTsMm 30480; H sapiens ADP ribosylation factor GTPase activating protein 1; Mus elongation of very long chain fatty acids; Mouse Y box binding protein 1 DNA binding MSY 1; Homo sapiens KIAA0249 gene product; Mus musculus Ran binding protein 2; Mus musculus histidine decarboxylase cluster; Homo sapiens cDNA FLJ21612 fis clone COL07355; UI M BH2 3 aqc g 10 0 UI 5; Rattus norvegicus APP binding protein 1; Mus musculus beta site APP cleaving enzyme; DNA methyltransferase cytosine 5 Mm 7814; no match66; ESTs Weakly similar to Lpi2p S cerevisiae Mm 21859; R norvegicus phosphatidylinositol synthase; ribonuclease L 2 5 oligoisoadenylate synthetase dependent inhibitorMm 5831; Mm 104074; H sapiens protein phosphatase 2A regulatory subunit B; H3147A11 5 NIA Mouse 15K cDNA Clone Set; Mus musculus Y box transcription factor; Mouse gene for basigin; Homo sapiens mRNA for FLJ00042 protein; R norvegicus nup155 nucleoporin 155kD; tubby like protein 1 Mm 42102; R norvegicus RNA binding protein SiahBP; UI M BZ0 axj h 06 0 UI 3; and Mus musculus pyruvate kinase 3 is administered to the mammal. Neuronal cell death in the mammal is thereby inhibited or prevented.

A fourth embodiment of the invention is a method of identifying regions of neuronal cell death in a patient. A molecule comprising an antibody variable region is administered to the patient. The molecule is bound to a detectable moiety. The antibody variable region specifically binds to a NM protein selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculu's secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258, ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6. The detectable moiety in the pateint is detected. Regions of neuronal cell death are thereby detected.

A fifth embodiment of the invention is a method of screening for neuronal cell death in a patient. A body fluid collected from the patient is contacted with a molecule comprising an antibody variable region which specifically binds to a NM protein selected from the group consisting of: ESTsMm 40262; Mus musculus calcium binding protein 1; :M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6. Detection of cross-reactive material in the body fluid with the molecule indicates neuronal cell death in the patient.

A sixth embodiment of the invention is method for promoting neuronal cell death in a patient. An NM protein selected from the group consising of: ESTsMm 40262; Mus musculus calcium binding protein l; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6 is administered to the patient. Neuronal cell death in the patient is thereby stimulated.

A seventh embodiment of the invention is a method of promoting neuronal cell death in a patient. A nucleic acid molecule encoding a NM protein is administered to the patient. The NM protein is selected from the group consising of ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6. The NM protein is expressed in the patient and neuronal cell death in the patient is thereby stimulated.

An eighth embodiment of the invention is a method of screening for neuronal cell death in a patient. An NM protein is detected in a body fluid collected from the patient. The NM protein is selected from the group consisting of ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5, Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculusG protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6. Detection of the NM protein indicates neuronal cell death in the patient.

A ninth embodiment of the invention is a method of screening for neuronal cell death in a patient. A nucleic acid encoding an NM protein selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6 is detected in a body fluid of the patient. Detection of the NM protein indicates neuronal cell death in the patient.

A tenth embodiment of the invention is a method to identify candidate drugs for treating neuronal cell death. Cells which express one or more NM genes are contacted with a test compound. The NM genes are selected from the group consisting of ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein I54; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match7l; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344, R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 2746; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6. Expression of said one or more NM genes is detected by hybridization of mRNA of said cells to a nucleic acid probe which is complementary to said mRNA. A test compound is identified as a candidate drug for treating neuronal cell death if it decreases expression of said one or more NM genes.

An eleventh embodiment of the invention is a method to identify candidate drugs for treating neuronal cell death. Cells which express one or more NM proteins are contacted with a test compound. The NM proteins are selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER I M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6. The amount of said one or more NM proteins in said cells is determined. A test compound is identified as a candidate drug for treating tumors if it decreases the amount of one or more NM proteins in said cells.

An eleventh embodiment of the invention is a method to identify candidate drugs for treating neuronal cell death. Cells which express one or more NM proteins are contacted with a test compound. The NM proteins are selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type m; heat shock protein 25 kDa 2 cardiovascular Mm 103612; Mus musculus hypothetical protein I54; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6. Activity of said one or more NM proteins in said cells is determined. A test compound is identified as a candidate drug for treating neuronal cell death if it decreases the activity of one more NM proteins in said cells.

A twelfth embodiment of the invention is a method to identify candidate drugs for treating neuronal cell death. Cells are contacted with a test compound. The cells express one or more NM genes selected from the group consisting of Mus musculus retinal S antigen; Mus musculus neural retina leucine zipper gene; M musculus photoreceptor specific protein PSP G145; IMAGE 4507893 5; Mus musculus domesticus phosducin; IMAGE 4507284 5; Danio rerio brain type fatty acid binding protein; M musculus X linked juvenile retinoschisis protein; M musculus guanine nucleotide binding protein beta 1 Gnb1; Mus musculus TPA regulated locus; Mouse nuclear protein mdm 1; IMAGE 4511806 5; M musculus male germ cell associated kinase; heat shock protein 60 kDaMm 1777; no match17; NCI CGAP BC3 Mus musculus cDNA clone IMAGE 3976794; no homo16; Homo sapiens CGI 45 protein; ESTsMm 44103; Mouse opsin MOPS; IMAGE 4225062 5; Mm 100212; H sapiens fer fps fes related tyrosine kinase phosphoprotein NCP94 FER; IMAGE 4505626 5 602393946F1 NIH MGC 94; solute carrier family 12 member 2Mm 4168; Mus musculus BUB2 like protein 1 HBLP1 mRNA complete cdsMm 104771; hemoglobin Y beta like embryonic chainMm 35830; erythrocyte protein band 4 1Mm 30038; no match55; Mus musculus MYLE protein mRNA complete cdsMm 41091; RIKEN full length enriched adult male hypothalamus musculus cDNA clone A230050E13; NCI CGAP Mam6 Mus IMAGE 3500058; Mus musculus mRNA for GTP binding protein drg2 gene Mm 41803; Homo sapiens mRNA for KLAA1549 protein; Mus musculus karyopherin importin alpha 2 Kpna2; UI M BZ1 bkv b 01 0 UI 3; no match B; ESTsMm 939; Mus musculus cDNA sequence AF244542; IMAGE 1348390 5; solute carrier family 30 zinc transporter member 3Mm 1396; no match110; Mus musculus homeodomain protein crx; promininMm 6250; no homo13; IMAGE 1279184 5; Human microfibril associated glycoprotein 4; Mm 70462; no match A; Rattus sp mRNA for BHF 1; ribosomal protein S24Mm 16775; Stratagene mouse Tcell 937311 IMAGE 1002041; NCI CGAP Kid14 Mus IMAGE 4236354 5; R norvegicus retinoblastoma binding protein 9; Mus musculus exostoses multiple 1 Ext1; selectin endothelial cell ligandMm 488; ESTs Weakly similar to HYPOTHETICAL 16 1 KD PROTEIN IN SEC17 QCR1 INTERGENIC REGION Saccharomyces cerevisiae Mm 27114; ESTs Highly similar to KIAA0824 protein H sapiens Mm 34579; Mus musculus ribospsmal protein L10A Rp110a; R norvegicus ribonucleoprotein F; clone 11 10007F23; no match38; M musculus Srp20 gene; homeodomain interacting protein kinase 2Mm 20934; FSHD region gene 1Mm 67; UI M BH3 ari c 10 0 UI s1 NIH BMAP M S4; Homo sapiens CED 6 protein CED 6; Mus musculus RIKEN clone 0610009E22; RAB18 member RAS oncogene familyMm 22660; no match5; Mus musculus prominin Prom; ribosomal protein L12Mm 70127; ESTs Highly similar to ELONGATION FACTOR 1 DELTA Homo sapiens Mm 21086; ESTs Highly similar to HYPOTHETICAL 37 2 KD PROTEIN C12C2 09C IN CHROMOSOME I Schizosaccharomyces pombe Mm 21383; clone 3021401C12; M musculus very long chain acyl CoA dehydrogenase; vitronectinMm 3667; ESTs Weakly similar to LIV 1 protein H sapiens Mm 41214; Mus musculus dopamine receptor 4; no match7; ATPase H transporting lysosomal vacuolar proton pump noncatalytic accessory protein 1 110 160 kDa Mm 20869; Rattus norvegicus partial mRNA for CRM1 protein; eukaryotic translation elongation factor 1 alpha 1Mm 16317; Human karyopherin beta2 importin; ESTs Moderately similar to hypothetical protein H sapiens Mm 22878; Homo sapiens PAC clone RP4 687K1; UI M AO1 aeh e 11 0 UI r1 NIH BMAP MPG N; high mobility group protein 14Mm 2756; ESTsMm 31374; R norvegicus aryl hydrocarbon interacting protein like 1; UI M CG0p bmu h 08 0 UI s1 NIH BMAP Ret4 S2; RAB10 member RAS oncogene familyMm 9455; Mus musculus early development regulator 2; no match83; Mus musculus topoisomerase DNA II beta; alpha tubulin; Homo sapiens MTA1 L1; retinitis pigmentosa GTPase regulator interacting protein 1 Mm 21662; Mus musculus FXYD dom containing ion transport regulator 5; Mus musculus cytochrome P450 3A25 CYP3A25 mRNA complete cdsMm 26993; IMAGE 4505626 5; RNA polymerase II transcriptional coactivatorMm 966; ESTs Highly similar to CAAX prenyl protease H sapiens Mm 34399; Soares mammary gland NbMMG IMAGE 1347586; clone 2700067D09; ESTs Weakly similar to defline not available 5901802 D melanogaster Mm 35127; torsin family 1 member AMm 29151; Mm 23086; M musculus brain cyclic nucleotide gated K; Mus musculus N myc downstream regulated 1; Homo sapiens splicing factor 3b subunit 3; Mus musculus mRNA for Lim homeodomain protein Islet1Mm 42242; Mouse mRNA for syntaxin 3D 1; Mus musculus chromosome 7 clone 19K5; ES18 proteinMm 23296; ESTs Highly similar to KIAA0729 protein H sapiens Mm 13148; ESTsMm 33949; Rat transcription factor RZR beta gene; ESTs Moderately similar to hypothetical protein H sapiens Mm 30235; Homo sapiens KIAA0009 gene product; no match X; ESTs Moderately similar to MYOSIN LIGHT CHAIN KINASE Dictyostelium discoideum Mm 1881; serum glucocorticoid regulated kinaseMm 28405; ESTs Weakly similar to cappuccino D melanogaster Mm 41762; regulator of G protein signaling 9Mm 38548; ESTsMm 34351; ESTsMm 32460; Mm 44404; ESTsMm 37515; Mus musculus cytochrome P450 2f2 Cyp2f2; Finkel Biskis Reilly murine sarcoma virus FBR MuSV ubiquitously expressed fox derived Mm 4890; guanylate cyclase activator 1a retina Mm 16224; human CRX control; adducin 2 beta Mm 104155; mouse CRX control; NRL control; Mus musculus ELOVL4; Mus musculus N myc downstream regulated 3; lactate dehydrogenase 1 A chainMm 26504; ESTs Moderately similar to stromelysin PDGF responsive element binding protein transcription factor M musculus Mm 38372; ESTsMm 11285; M musculus chr 10 clone RP21 39C4; ESTs Highly similar to 40 KD PEPTIDYL PROLYL CIS TRANS ISOMERASE Homo sapiens Mm 30242; NIH BMAP Ret4 S2 Mus UI M CG0p big e 08 0 UI 3; Soares mammary gland NMLMG IMAGE 3467149; glycosylphosphatidylinositol 1 homolog human Mm 6354; Rattus norvegicus NMDA receptor subunit NR2; ESTsMm 33788; Mus musculus hexokinase 1 Hk1; inosine 5 phosphate dehydrogenase 2Mm 6065; N myc downstream regulated 3Mm 36775; no match V; villin 2Mm 4551; Rattus norvegicus TM6P1 TM6P1; Mus musculus mRNA for heterogeneous nuclear ribonucleoprotein HMm 21740; ESTsMm 103333; Mus musculus retinal taurine transporter; Mus musculus poly rC binding protein; ESTs Weakly similar to nuclear poly C binding protein M musculus Mm 29707; ESTs Weakly similar to similar to 1 acyl glycerol 3 phosphate acyltransferases C elegans Mm 24117; Mm 27013; pre B cell leukemia transcription factor 3Mm 7331; ESTsMm 21299; Mus musculus kinectin 1; Mus musculus drebrin A mRNA complete cdsMm 104044; H3087H01 5 NIA Mouse 15K cDNA Clone Set; SAC483 Mouse e14 5 developing pituitary gland; cloneE130113K08; Mus musculus major histocompatibility locus class II region Fas binding protein Daxx DAXX gene partial cds Bing1 BING1 tapasin tapasin RalGDS like factor RLF KE2 KE2 BING4 BING4 beta1 3 galactosyl transferase beta1 3 galactosylMm 20926; Mus musculus aquaporin 1; acyl Coenzyme A dehydrogenase very long chainMm 18630; Mouse proprotein convertase 4; M musculus activating transcription factor 4 Atf4; guanine nucleotide binding protein beta 5Mm 4702; phosducin control; ESTsMm 38578; Barstead bowel MPLRB9 IMAGE 1095982; M musculus stromal cell derived factor recep; ESTs Weakly similar to E04F6 2 gene product C elegans Mm 18889; IMAGE 963149 5; syntaxin binding protein 1 Mm 3129; solute carrier family 16 monocarboxylic acid transporters member 1Mm 9086; ESTs Highly similar to TRICARBOXYLATE TRANSPORT PROTEIN PRECURSOR Rattus norvegicus Mm 22679; Bcl2 likeMm 3882; Soares mouse p3NMF19 5 IMAGE 493296; Mus musculus beta galactosidase complex; H sapiens ADP ribosylation factor binding protein GGA2; Mm 31266; IMAGE 560050 5; Mus musculus DXHXS6673E protein DXHXS6673E mRNA complete cdsMm 23458; M musculus mRNA for hair keratin mHb6; Mus musculus thyroglobulin; ESTs Moderately similar to KIAA0956 protein H sapiens Mm 11428; H3050H05 3 NIA Mouse 15K cDNA Clone Set; ESTs Moderately similar to signal recognition particle 54K protein M musculus Mm 32508; Mouse PSD 95 SAP90A; ESTsMm 29308; alkaline phosphatase 2 liverMm 1265; Homo sapiens 12 seeders BAC RP11 19E18; ESTsMm 41269; ESTsMm 86724; Homo sapiens 12q13 1 PAC RPCI1 228P16; serine threonine kinase receptor associated proteinMm 22584; UI M BZ0 axl a 11 0 UI s1 NIH BMAP MHI2; Mus musculus poly rC binding protein 2; IMAGE 4503171 5; ESTsMm 35430; activating transcription factor 4Mm 641; Mouse serine threonine phosphatase 2C; GAPDH control; Human mRNA for KIAA0299; ESTs Weakly similar to proline rich protein M musculus Mm 41665; megakaryocyte associated tyrosine kinaseMm 2918; homer neuronal immediate early gene 2Mm 228; peroxisomal farnesylated proteinMm 29198; blank; zinc finger protein 238Mm 27962; ESTs Highly similar to PHENYLALANYL TRNA SYNTHETASE BETA CHAIN CYTOPLASMIC Saccharomyces cerevisiae Mm 27403; Rat microtubule associated protein 2 MAP2; timeless homolog Drosophila Mm 6458; kinectin 1Mm 3110; phosphatidylinositol membrane associatedMm 1860; R norvegicus CDP diacylglycerol synthase; Homo sapiens DKFZp434A132; Mus musculus hematopoietic zinc finger; mitogen activated protein kinase kinase 7Mm 3906; H3110G03 3 NIA Mouse 15K cDNA; ESTs Highly similar to HYPOTHETICAL 47 9 KD PROTEIN B0303 3 IN CHROMOSOME III Caenorhabditis elegans Mm 30147; ESTs Highly similar to CELL GROWTH REGULATING NUCLEOLAR PROTEIN M musculus Mm 28560; no match W; Mouse endogenous murine leukemia virus polytropic provirus DNA; clone1110013A05; aryl hydrocarbon receptorMm 4452; peroxisome proliferator activated receptor alphaMm 1373; Mus musculus LAG protein Lag Rattus NMDA receptor glutamate binding subunit; Mus musculus syntaxin binding protein 1; Mus musculus MAP kinase phosphatase 6; Rattus norvegicus retina specific protein PAL; no match33; Mus musculus myc box dependent interacting pro; Murine leukemia virus erv1 envelope protein; cytochrome c oxidase subunit VIIa 3Mm 2151; proteasome prosome macropain subunit alpha type 3Mm 1007; Homo sapiens mRNA cDNA DKFZp434N1615; Mus musculus TCR beta locus; ESTs Weakly similar to LOK M musculus Mm 74661; small inducible cytokine subfamily A member 22Mm 12895; ESTsMm 23682; no match I; no match H; high mobility group protein I isoform CMm 3953; protein kinase cAMP dependent catalytic alphaMm 22479; Mus musculus phosphatidylinositol membrane associated; no match G; Mouse heparin binding epidermal growth factor like; Homo sapiens cDNA DKFZp586B0924; Mouse magnesium dependent protein; ESTs Weakly similar to ZW10 interactor Zwint H sapiens Mm 38994; ESTsMm 30480; H sapiens ADP ribosylation factor GTPase activating protein 1; Mus elongation of very long chain fatty acids; Mouse Y box binding protein 1 DNA binding MSY 1; Homo sapiens KIAA0249 gene product; Mus musculus Ran binding protein 2; Mus musculus histidine decarboxylase cluster; Homo sapiens cDNA FLJ21612 fis clone COL07355; UI M BH2 3 aqc g 10 0 UI 5; Rattus norvegicus APP binding protein 1; Mus musculus beta site APP cleaving enzyme; DNA methyltransferase cytosine 5 Mm 7814; no match66; ESTs Weakly similar to Lpi2p S cerevisiae Mm 21859; R norvegicus phosphatidylinositol synthase; ribonuclease L 2 5 oligoisoadenylate synthetase dependent inhibitorMm 5831; Mm 104074; H sapiens protein phosphatase 2A regulatory subunit B; H3147A11 5 NIA Mouse 15K cDNA Clone Set; Mus musculus Y box transcription factor; Mouse gene for basigin; Homo sapiens mRNA for FLJ00042 protein; R norvegicus nup155 nucleoporin 155kD; tubby like protein 1 Mm 42102; R norvegicus RNA binding protein SiahBP; UI M BZ0 axj h 06 0 UI 3; and Mus musculus pyruvate kinase 3. Expression of said one or more N\M genes is detected by hybridization of mRNA of said cells to a nucleic acid probe which is complementary to said mRNA. A test compound is identified as a candidate drug for treating neuronal cell death if it increases expression of said one or more NM genes.

A thirteenth embodiment of the invention is a method for identifying candidate drugs for treating neuronal cell death. Cells which express one or more NM proteins are contacted with a test compound. The NM proteins are selected from the group consisting of: Mus musculus retinal S antigen; Mus musculus neural retina leucine zipper gene; M musculus photoreceptor specific protein PSP G145; IMAGE 4507893 5; Mus musculus domesticus phosducin; IMAGE 4507284 5; Danio rerio brain type fatty acid binding protein; M musculus X linked juvenile retinoschisis protein; M musculus guanine nucleotide binding protein beta 1 Gnb1; Mus musculus TPA regulated locus; Mouse nuclear protein mdm 1; IMAGE 4511806 5; M musculus male germ cell associated kinase; heat shock protein 60 kDaMm 1777; no match17; NCI CGAP BC3 Mus musculus cDNA clone IMAGE 3976794; no homo16; Homo sapiens CGI 45 protein; ESTsMm 44103; Mouse opsin MOPS; IMAGE 4225062 5; Mm 100212; H sapiens fer fps fes related tyrosine kinase phosphoprotein NCP94 FER; IMAGE 4505626 5 602393946F1 NIH MGC 94; solute carrier family 12 member 2Mm 4168; Mus musculus BUB2 like protein 1 HBLP1 mRNA complete cdsMm 104771; hemoglobin Y beta like embryonic chainMm 35830; erythrocyte protein band 4 1Mm 30038; no match55; Mus musculus MYLE protein mRNA complete cdsMm 41091; RIKEN full length enriched adult male hypothalamus musculus cDNA clone A230050E13; NCI CGAP Mam6 Mus IMAGE 3500058; Mus musculus mRNA for GTP binding protein drg2 gene Mm 41803; Homo sapiens mRNA for KIAA1549 protein; Mus musculus karyopherin importin alpha 2 Kpna2; UI M BZ1 bkv b 01 0 UI 3; no match B; ESTsMm 939; Mus musculus cDNA sequence AF244542; IMAGE 1348390 5; solute carrier family 30 zinc transporter member 3Mm 1396; no match110; Mus musculus homeodomain protein crx; promininMm 6250; no homo13; IMAGE 1279184 5; Human microfibril associated glycoprotein 4; Mm 70462; no match A; Rattus sp mRNA for BHF 1; ribosomal protein S24Mm 16775; Stratagene mouse Tcell 937311 IMAGE 1002041; NCI CGAP Kid14 Mus IMAGE 4236354 5; R norvegicus retinoblastoma binding protein 9; Mus musculus exostoses multiple 1 Ext1; selectin endothelial cell ligandMm 488; ESTs Weakly similar to HYPOTHETICAL 16 1 KD PROTEIN IN SEC17 QCR1 INTERGENIC REGION Saccharomyces cerevisiae Mm 27114; ESTs Highly similar to KIAA0824 protein H sapiens Mm 34579; Mus musculus ribospsmal protein L10A Rp110a; R norvegicus ribonucleoprotein F; clone 1110007F23; no match38; M musculus Srp20 gene; homeodomain interacting protein kinase 2Mm 20934; FSHD region gene 1Mm 67; UI M BH3 ari c 10 0 UI s1 NIH BMAP M S4; Homo sapiens CED 6 protein CED 6; Mus musculus RIKEN clone 0610009E22; RAB18 member RAS oncogene familyMm 22660; no match5; Mus musculus prominin Prom; ribosomal protein L12Mm 70127; ESTs Highly similar to ELONGATION FACTOR 1 DELTA Homo sapiens Mm 21086; ESTs Highly similar to HYPOTHETICAL 37 2 KD PROTEIN C12C2 09C IN CHROMOSOME I Schizosaccharomyces pombe Mm 21383; clone 3021401C12; M musculus very long chain acyl CoA dehydrogenase; vitronectinMm 3667; ESTs Weakly similar to LIV 1 protein H sapiens Mm 41214; Mus musculus dopamine receptor 4; no match7; ATPase H transporting lysosomal vacuolar proton pump noncatalytic accessory protein 1 110 160 kDa Mm 20869; Rattus norvegicus partial mRNA for CRM1 protein; eukaryotic translation elongation factor 1 alpha 1Mm 16317; Human karyopherin beta2 importin; ESTs Moderately similar to hypothetical protein H sapiens Mm 22878; Homo sapiens PAC clone RP4 687K1; UI M AO1 aeh e 11 0 UI r1 NIH BMAP MPG N; high mobility group protein 14Mm 2756; ESTsMm 31374; R norvegicus aryl hydrocarbon interacting protein like 1; UI M CG0p bmu h 08 0 UI s1 NIH BMAP Ret4 S2; RAB10 member RAS oncogene familyMm 9455; Mus musculus early development regulator 2; no match83; Mus musculus topoisomerase DNA II beta; alpha tubulin; Homo sapiens MTA1 L1; retinitis pigmentosa GTPase regulator interacting protein 1 Mm 21662; Mus musculus FXYD dom containing ion transport regulator 5; Mus musculus cytochrome P450 3A25 CYP3A25 mRNA complete cdsMm 26993; IMAGE 4505626 5; RNA polymerase II transcriptional coactivatorMm 966; ESTs Highly similar to CAAX prenyl protease H sapiens Mm 34399; Soares mammary grand NbMMG IMAGE 1347586; clone 2700067D09; ESTs Weakly similar to defline not available 5901802 D melanogaster Mm 35127; torsin family 1 member AMm 29151; Mm 23086; M musculus brain cyclic nucleotide gated K; Mus musculus N myc downstream regulated 1; Homo sapiens splicing factor 3b subunit 3; Mus musculus mRNA for Lim homeodomain protein Islet1Mm 42242; Mouse mRNA for syntaxin 3D 1; Mus musculus chromosome 7 clone 19K5; ES18 proteinMm 23296; ESTs Highly similar to KIAA0729 protein H sapiens Mm 13148; ESTsMm 33949; Rat transcription factor RZR beta gene; ESTs Moderately similar to hypothetical protein H sapiens Mm 30235; Homo sapiens KIAA0009 gene product; no match X; ESTs Moderately similar to MYOSIN LIGHT CHAIN KINASE Dictyostelium discoideum Mm 1881; serum glucocorticoid regulated kinaseMm 28405; ESTs Weakly similar to cappuccino D melanogaster Mm 41762; regulator of G protein signaling 9Mm 38548; ESTsMm 34351; ESTsMm 32460; Mm 44404; ESTsMm 37515; Mus musculus cytochrome P450 2f2 Cyp2f2; Finkel Biskis Reilly murine sarcoma virus FBR MuSV ubiquitously expressed fox derived Mm 4890; guanylate cyclase activator 1a retina Mm 16224; human CRX control; adducin 2 beta Mm 104155; mouse CRX control; NRL control; Mus musculus ELOVL4; Mus musculus N myc downstream regulated 3; lactate dehydrogenase 1 A chainMm 26504; ESTs Moderately similar to stromelysin PDGF responsive element binding protein transcription factor M musculus Mm 38372; ESTsMm 11285; M musculus chr 10 clone RP21 39C4; ESTs Highly similar to 40 KD PEPTIDYL PROLYL CIS TRANS ISOMERASE Homo sapiens Mm 30242; NIH BMAP Ret4 S2 Mus UI M CG0P big e 08 0 UI 3; Soares mammary gland NMLMG IMAGE 3467149; glycosylphosphatidylinositol 1 homolog human Mm 6354; Rattus norvegicus NMDA receptor subunit NR2; ESTsMm 33788; Mus musculus hexokinase 1 Hk1; inosine 5 phosphate dehydrogenase 2Mm 6065; N myc downstream regulated 3Mm 36775; no match V; villin 2Mm 4551; Rattus norvegicus TM6P1 TM6P1; Mus musculus mRNA for heterogeneous nuclear ribonucleoprotein HMm 21740; ESTsMm 103333; Mus musculus retinal taurine transporter; Mus musculus poly rC binding protein; ESTs Weakly similar to nuclear poly C binding protein M musculus Mm 29707; ESTs Weakly similar to similar to 1 acyl glycerol 3 phosphate acyltransferases C elegans Mm 24117; Mm 27013; pre B cell leukemia transcription factor 3Mm 7331; ESTsMm 21299; Mus musculus kinectin 1; Mus musculus drebrin A mRNA complete cdsMm 104044; H3087H01 5 NIA Mouse 15K cDNA Clone Set; SAC483 Mouse e14 5 developing pituitary gland; cloneE130113K08; Mus musculus major histocompatibility locus class II region Fas binding protein Daxx DAXX gene partial cds Bing1 BING1 tapasin tapasin RalGDS like factor RLF KE2 KE2 BING4 BING4 beta1 3 galactosyl transferase beta1 3 galactosylMm 20926; Mus musculus aquaporin 1; acyl Coenzyme A dehydrogenase very long chainMm 18630; Mouse proprotein convertase 4; M musculus activating transcription factor 4 Atf4; guanine nucleotide binding protein beta 5Mm 4702; phosducin control; ESTsMm 38578; Barstead bowel MPLRB9 IMAGE 1095982; M musculus stromal cell derived factor recep; ESTs Weakly similar to E04F6 2 gene product C elegans Mm 18889; IMAGE 963149 5; syntaxin binding protein 1 Mm 3129; solute carrier family 16 monocarboxylic acid transporters member 1Mm 9086; ESTs Highly similar to TRICARBOXYLATE TRANSPORT PROTEIN PRECURSOR Rattus norvegicus Mm 22679; Bcl2 likeMm 3882; Soares mouse p3NMF19 5 IMAGE 493296; Mus musculus beta galactosidase complex; H sapiens ADP ribosylation factor binding protein GGA2; Mm 31266; IMAGE 560050 5; Mus musculus DXHXS6673E protein DXHXS6673E mRNA complete cdsMm 23458; M musculus mRNA for hair keratin mHb6; Mus musculus thyroglobulin; ESTs Moderately similar to KIAA0956 protein H sapiens Mm 11428; H3050H05 3 NIA Mouse 15K cDNA Clone Set; ESTs Moderately similar to signal recognition particle 54K protein M musculus Mm 32508; Mouse PSD 95 SAP90A; ESTsMm 29308; alkaline phosphatase 2 liverMm 1265; Homo sapiens 12 seeders BAC RP11 19E18; ESTsMm 41269; ESTsMm 86724; Homo sapiens 12q13 1 PAC RPCI1 228P16; serine threonine kinase receptor associated proteinMm 22584; UI M BZ0 axl a 11 0 UI s1 NIH BMAP MHI2; Mus musculus poly rC binding protein 2; IMAGE 4503171 5; ESTsMm 35430; activating transcription factor 4Mm 641; Mouse serine threonine phosphatase 2C; GAPDH control; Human mRNA for KIAA0299; ESTs Weakly similar to proline rich protein M musculus Mm 41665; megakaryocyte associated tyrosine kinaseMm 2918; homer neuronal immediate early gene 2Mm 228; peroxisomal farnesylated proteinMm 29198; blank; zinc finger protein 238Mm 27962; ESTs Highly similar to PHENYLALANYL TRNA SYNTHETASE BETA CHAIN CYTOPLASMIC Saccharomyces cerevisiae Mm 27403; Rat microtubule associated protein 2 MAP2; timeless homolog Drosophila Mm 6458; kinectin 1Mm 3110; phosphatidylinositol membrane associatedMm 1860; R norvegicus CDP diacylglycerol synthase; Homo sapiens DKFZp434A132; Mus musculus hematopoietic zinc finger; mitogen activated protein kinase kinase 7Mm 3906; H3110G03 3 NIA Mouse 15K cDNA; ESTs Highly similar to HYPOTHETICAL 47 9 KD PROTEIN B0303 3 IN CHROMOSOME III Caenorhabditis elegans Mm 30147; ESTs Highly similar to CELL GROWTH REGULATING NUCLEOLAR PROTEIN M musculus Mm 28560; no match W; Mouse endogenous murine leukemia virus polytropic provirus DNA; clone1110013A05; aryl hydrocarbon receptorMm 4452; peroxisome proliferator activated receptor alphaMm 1373; Mus musculus LAG protein Lag Rattus NMDA receptor glutamate binding subunit; Mus musculus syntaxin binding protein 1; Mus musculus MAP kinase phosphatase 6; Rattus norvegicus retina specific protein PAL; no match33; Mus musculus myc box dependent interacting pro; Murine leukemia virus erv1 envelope protein; cytochrome c oxidase subunit VIIa 3Mm 2151; proteasome prosome macropain subunit alpha type 3Mm 1007; Homo sapiens mRNA cDNA DKFZp434N1615; Mus musculus TCR beta locus; ESTs Weakly similar to LOK M musculus Mm 74661; small inducible cytokine subfamily A member 22Mm 12895; ESTsMm 23682; no match I; no match H; high mobility group protein I isoform CMm 3953; protein kinase cAMP dependent catalytic alphaMm 22479; Mus musculus phosphatidylinositol membrane associated; no match G; Mouse heparin binding epidermal growth factor like; Homo sapiens cDNA DKFZp586B0924; Mouse magnesium dependent protein; ESTs Weakly similar to ZW10 interactor Zwint H sapiens Mm 38994; ESTsMm 30480; H sapiens ADP ribosylation factor GTPase activating protein 1; Mus elongation of very long chain fatty acids; Mouse Y box binding protein 1 DNA binding MSY 1; Homo sapiens KIAA0249 gene product; Mus musculus Ran binding protein 2; Mus musculus histidine decarboxylase cluster; Homo sapiens cDNA FLJ21612 fis clone COL07355; UI M BH2 3 aqc g 10 0 UI 5; Rattus norvegicus APP binding protein 1; Mus musculus beta site APP cleaving enzyme; DNA methyltransferase cytosine 5 Mm 7814; no match66; ESTs Weakly similar to Lpi2p S cerevisiae Mm 21859; R norvegicus phosphatidylinositol synthase; ribonuclease L 2 5 oligoisoadenylate synthetase dependent inhibitorMm 5831; Mm 104074; H sapiens protein phosphatase 2A regulatory subunit B; H3147A1 1 5 NIA Mouse 15K cDNA Clone Set; Mus musculus Y box transcription factor; Mouse gene for basigin; Homo sapiens mRNA for FLJ00042 protein; R norvegicus nup155 nucleoporin 155kD; tubby like protein 1 Mm 42102; R norvegicus RNA binding protein SiahBP; UI M BZ0 axj h 06 0 UI 3; and Mus musculus pyruvate kinase 3. The amount of said one or more NM proteins in said cells is determined. A test compound is identified as a candidate drug for treating neuronal cell death if it increases the amount of one more NM proteins in said cells.

A fourteenth embodiment of the invention is a method to identify candidate drugs for treating neuronal cell death. Cells are contacted with a test compound. The cells express one or more NM proteins selected from the group consisting of: Mus musculus retinal S antigen; Mus musculus neural retina leucine zipper gene; M musculus photoreceptor specific protein PSP G145; IMAGE 4507893 5; Mus musculus domesticus phosducin; IMAGE 4507284 5; Danio rerio brain type fatty acid binding protein; M musculus X linked juvenile retinoschisis protein; M musculus guanine nucleotide binding protein beta 1 Gnb1; Mus musculus TPA regulated locus; Mouse nuclear protein mdm 1; IMAGE 4511806 5; M musculus male germ cell associated kinase; heat shock protein 60 kDaMm 1777; no match17; NCI CGAP BC3 Mus musculus cDNA clone IMAGE 3976794; no homo16; Homo sapiens CGI 45 protein; ESTsMm 44103; Mouse opsin MOPS; IMAGE 4225062 5; Mm 100212; H sapiens fer fps fes related tyrosine kinase phosphoprotein NCP94 FER; IMAGE 4505626 5 602393946F1 NIH MGC 94; solute carrier family 12 member 2Mm 4168; Mus musculus BUB2 like protein 1 HBLP1 mRNA complete cdsMm 104771; hemoglobin Y beta like embryonic chainMm 35830; erythrocyte protein band 4 1Mm 30038; no match55; Mus musculus MYLE protein mRNA complete cdsMm 41091; RIKEN full length enriched adult male hypothalamus musculus cDNA clone A230050E13; NCI CGAP Mam6 Mus IMAGE 3500058; Mus musculus mRNA for GTP binding protein drg2 gene Mm 41803; Homo sapiens mRNA for KIAA1549 protein; Mus musculus karyopherin importin alpha 2 Kpna2; UI M BZ1 bkv b 01 0 UI 3; no match B; ESTsMm 939; Mus musculus cDNA sequence AF244542; IMAGE 1348390 5; solute carrier family 30 zinc transporter member 3Mm 1396; no match110; Mus musculus homeodomain protein crx; promininMm 6250; no homo13; IMAGE 1279184 5; Human microfibril associated glycoprotein 4; Mm 70462; no match A; Rattus sp mRNA for BHF 1; ribosomal protein S24Mm 16775; Stratagene mouse Tcell 937311 IMAGE 1002041; NCI CGAP Kid14 Mus IMAGE 4236354 5; R norvegicus retinoblastoma binding protein 9; Mus musculus exostoses multiple 1 Ext1; selectin endothelial cell ligandMm 488; ESTs Weakly similar to HYPOTHETICAL 16 1 KD PROTEIN IN SEC17 QCR1 INTERGENIC REGION Saccharomyces cerevisiae Mm 27114; ESTs Highly similar to KIAA0824 protein H sapiens Mm 34579; Mus musculus ribospsmal protein L10A Rp110a; R norvegicus ribonucleoprotein F; clone 1110007F23; no match38; M musculus Srp20 gene; homeodomain interacting protein kinase 2Mm 20934; FSHD region gene 1Mm 67; UI M BH3 ari c 10 0 UI s1 NIH BMAP M S4; Homo sapiens CED 6 protein CED 6; Mus musculus RIKEN clone 0610009E22; RAB18 member RAS oncogene familyMm 22660; no match5; Mus musculus prominin Prom; ribosomal protein L12Mm 70127; ESTs Highly similar to ELONGATION FACTOR 1 DELTA Homo sapiens Mm 21086; ESTs Highly similar to HYPOTHETICAL 37 2 KD PROTEIN C12C2 09C IN CHROMOSOME I Schizosaccharomyces pombe Mm 21383; clone 3021401C12; M musculus very long chain acyl CoA dehydrogenase; vitronectinMm 3667; ESTs Weakly similar to LIV 1 protein H sapiens Mm 41214; Mus musculus dopamine receptor 4; no match7; ATPase H transporting lysosomal vacuolar proton pump noncatalytic accessory protein 1 110 160 kDa Mm 20869; Rattus norvegicus partial mRNA for CRM1 protein; eukaryotic translation elongation factor 1 alpha 1Mm 16317; Human karyopherin beta2 importin; ESTs Moderately similar to hypothetical protein H sapiens Mm 22878; Homo sapiens PAC clone RP4 687K1; UI M AO1 aeh e 11 0 UI r1 NIH BMAP MPG N; high mobility group protein 14Mm 2756; ESTsMm 31374; R norvegicus aryl hydrocarbon interacting protein like 1; UI M CG0p bmu h 08 0 UI s1 NIH BMAP Ret4 S2; RAB10 member RAS oncogene familyMm 9455; Mus musculus early development regulator 2; no match83; Mus musculus topoisomerase DNA II beta; alpha tubulin; Homo sapiens MTA1 L1; retinitis pigmentosa GTPase regulator interacting protein 1 Mm 21662; Mus musculus FXYD dom containing ion transport regulator 5; Mus musculus cytochrome P450 3A25 CYP3A25 mRNA complete cdsMm 26993; IMAGE 4505626 5; RNA polymerase II transcriptional coactivatorMm 966; ESTs Highly similar to CAAX prenyl protease H sapiens Mm 34399; Soares mammary gland NbMMG IMAGE 1347586; clone 2700067D09; ESTs Weakly similar to defline not available 5901802 D melanogaster Mm 35127; torsin family 1 member AMm 29151; Mm 23086; M musculus brain cyclic nucleotide gated K; Mus musculus N myc downstream regulated 1; Homo sapiens splicing factor 3b subunit 3; Mus musculus mRNA for Lim homeodomain protein Islet1Mm 42242; Mouse mRNA for syntaxin 3D 1; Mus musculus chromosome 7 clone 19K5; ES18 proteinMm 23296; ESTs Highly similar to KIAA0729 protein H sapiens Mm 13148; ESTsMm 33949; Rat transcription factor RZR beta gene; ESTs Moderately similar to hypothetical protein H sapiens Mm 30235; Homo sapiens KIAA0009 gene product; no match X; ESTs Moderately similar to MYOSIN LIGHT CHAIN KINASE Dictyostelium discoideum Mm 1881; serum glucocorticoid regulated kinaseMm 28405; ESTs Weakly similar to cappuccino D melanogaster Mm 41762; regulator of G protein signaling 9Mm 38548; ESTsMm 34351; ESTsMm 32460; Mm 44404; ESTsMm 37515; Mus musculus cytochrome P450 2f2 Cyp2f2; Finkel Biskis Reilly murine sarcoma virus FBR MuSV ubiquitously expressed fox derived Mm 4890; guanylate cyclase activator 1 a retina Mm 16224; human CRX control; adducin 2 beta Mm 104155; mouse CRX control; NRL control; Mus musculus ELOVL4; Mus musculus N myc downstream regulated 3; lactate dehydrogenase 1 A chainMm 26504; ESTs Moderately similar to stromelysin PDGF responsive element binding protein transcription factor M musculus Mm 38372; ESTsMm 11285; M musculus chr 10 clone RP21 39C4; ESTs Highly similar to 40 KD PEPTIDYL PROLYL CIS TRANS ISOMERASE Homo sapiens Mm 30242; NIH BMAP Ret4 S2 Mus UI M CG0p big e 08 0 UI 3; Soares mammary gland NMLMG IMAGE 3467149; glycosylphosphatidylinositol 1 homolog human Mm 6354; Rattus norvegicus NMDA receptor subunit NR2; ESTsMm 33788; Mus musculus hexokinase 1 Hk1; inosine 5 phosphate dehydrogenase 2Mm 6065; N myc downstream regulated 3Mm 36775; no match V; villin 2Mm 4551; Rattus norvegicus TM6P1 TM6P1; Mus musculus mRNA for heterogeneous nuclear ribonucleoprotein HMm 21740; ESTsMm 103333; Mus musculus retinal taurine transporter; Mus musculus poly rC binding protein; ESTs Weakly similar to nuclear poly C binding protein M musculus Mm 29707; ESTs Weakly similar to similar to 1 acyl glycerol 3 phosphate acyltransferases C elegans Mm 24117; Mm 27013; pre B cell leukemia transcription factor 3Mm 7331; ESTsMm 21299; Mus musculus kinectin 1; Mus musculus drebrin A mRNA complete cdsMm 104044; H3087H01 5 NIA Mouse 15K cDNA Clone Set; SAC483 Mouse e14 5 developing pituitary gland; cloneE130113K08; Mus musculus major histocompatibility locus class II region Fas binding protein Daxx DAXX gene partial cds Bing1 BING1 tapasin tapasin RalGDS like factor RLF KE2 KE2 BING4 BING4 beta1 3 galactosyl transferase beta1 3 galactosylMm 20926; Mus musculus aquaporin 1; acyl Coenzyme A dehydrogenase very long chainMm 18630; Mouse proprotein convertase 4; M musculus activating transcription factor 4 Atf4; guanine nucleotide binding protein beta 5Mm 4702; phosducin control; ESTsMm 38578; Barstead bowel MPLRB9 IMAGE 1095982; M musculus stromal cell derived factor recep; ESTs Weakly similar to E04F6 2 gene product C elegans Mm 18889; IMAGE 963149 5; syntaxin binding protein 1 Mm 3129; solute carrier family 16 monocarboxylic acid transporters member 1Mm 9086; ESTs Highly similar to TRICARBOXYLATE TRANSPORT PROTEIN PRECURSOR Rattus norvegicus Mm 22679; Bcl2 likeMm 3882; Soares mouse p3NMF19 5 IMAGE 493296; Mus musculus beta galactosidase complex; H sapiens ADP ribosylation factor binding protein GGA2; Mm 31266; IMAGE 560050 5; Mus musculus DXHXS6673E protein DXHXS6673E mRNA complete cdsMm 23458; M musculus mRNA for hair keratin mHb6; Mus musculus thyroglobulin; ESTs Moderately similar to KIAA0956 protein H sapiens Mm 11428; H3050H05 3 NIA Mouse 15K cDNA Clone Set; ESTs Moderately similar to signal recognition particle 54K protein M musculus Mm 32508; Mouse PSD 95 SAP90A; ESTsMm 29308; alkaline phosphatase 2 liverMm 1265; Homo sapiens 12 seeders BAC RP11 19E18; ESTsMm 41269; ESTsMm 86724; Homo sapiens 12q13 1 PAC RPCI1 228P16; serine threonine kinase receptor associated proteinMm 22584; UI M BZ0 axl a 11 0 UI s1 NIH BMAP MHI2; Mus musculus poly rC binding protein 2; IMAGE 4503171 5; ESTsMm 35430; activating transcription factor 4Mm 641; Mouse serine threonine phosphatase 2C; GAPDH control; Human mRNA for KIAA0299; ESTs Weakly similar to proline rich protein M musculus Mm 41665; megakaryocyte associated tyrosine kinaseMm 2918; homer neuronal immediate early gene 2Mm 228; peroxisomal farnesylated proteinMm 29198; blank; zinc finger protein 238Mm 27962; ESTs Highly similar to PHENYLALANYL TRNA SYNTHETASE BETA CHAIN CYTOPLASMIC Saccharomyces cerevisiae Mm 27403; Rat microtubule associated protein 2 MAP2; timeless homolog Drosophila Mm 6458; kinectin 1Mm 3110; phosphatidylinositol membrane associatedMm 1860; R norvegicus CDP diacylglycerol synthase; Homo sapiens DKFZp434A132; Mus musculus hematopoietic zinc finger; mitogen activated protein kinase kinase 7Mm 3906; H3110G03 3 NIA Mouse 15K cDNA; ESTs Highly similar to HYPOTHETICAL 47 9 KD PROTEIN B0303 3 IN CHROMOSOME III Caenorhabditis elegans Mm 30147; ESTs Highly similar to CELL GROWTH REGULATING NUCLEOLAR PROTEIN M musculus Mm 28560; no match W; Mouse endogenous murine leukemia virus polytropic provirus DNA; clone1110013A05; aryl hydrocarbon receptorMm 4452; peroxisome proliferator activated receptor alphaMm 1373; Mus musculus LAG protein Lag Rattus NMDA receptor glutamate binding subunit; Mus musculus syntaxin binding protein 1; Mus musculus MAP kinase phosphatase 6; Rattus norvegicus retina specific protein PAL; no match33; Mus musculus myc box dependent interacting pro; Murine leukemia virus erv1 envelope protein; cytochrome c oxidase subunit VIIa 3Mm 2151; proteasome prosome macropain subunit alpha type 3Mm 1007; Homo sapiens mRNA cDNA DKFZp434N1615; Mus musculus TCR beta locus; ESTs Weakly similar to LOK M musculus Mm 74661; small inducible cytokine subfamily A member 22Mm 12895; ESTsMm 23682; no match I; no match H; high mobility group protein I isoform CMm 3953; protein kinase cAMP dependent catalytic alphaMm 22479; Mus musculus phosphatidylinositol membrane associated; no match G; Mouse heparin binding epidermal growth factor like; Homo sapiens cDNA DKFZp586B0924; Mouse magnesium dependent protein; ESTs Weakly similar to ZW10 interactor Zwint H sapiens Mm 38994; ESTsMm 30480; H sapiens ADP ribosylation factor GTPase activating protein 1; Mus elongation of very long chain fatty acids; Mouse Y box binding protein 1 DNA binding MSY 1; Homo sapiens KIAA0249 gene product; Mus musculus Ran binding protein 2; Mus musculus histidine decarboxylase cluster; Homo sapiens cDNA FLJ21612 fis clone COL07355; UI M BH2 3 aqc g 10 0 UI 5; Rattus norvegicus APP binding protein 1; Mus musculus beta site APP cleaving enzyme; DNA methyltransferase cytosine 5 Mm 7814; no match66; ESTs Weakly similar to Lpi2p S cerevisiae Mm 21859; R norvegicus phosphatidylinositol synthase; ribonuclease L 2 5 oligoisoadenylate synthetase dependent inhibitorMm 5831; Mm 104074; H sapiens protein phosphatase 2A regulatory subunit B; H3147AI 1 5 NIA Mouse 15K cDNA Clone Set; Mus musculus Y box transcription factor; Mouse gene for basigin; Homo sapiens mRNA for FLJ00042 protein; R norvegicus nup155 nucleoporin 155kD; tubby like protein 1 Mm 42102; R norvegicus RNA binding protein SiahBP; UI M BZ0 axj h 06 0 UI 3; and Mus musculus pyruvate kinase 3. Activity of said one or more NM proteins in said cells is determined. A test compound is identified as a candidate drug for treating neuronal cell death if it increases the activity of one more NM proteins in said cells.

These and other embodiments which will be apparent to those of skill in the art upon reading the specification provide the art with reagents and methods for detection, diagnosis, therapy, and drug screening pertaining to neuronal cell death and pathological processes involving or requiring neuronal cell death.

### BRIEF DESCRIPTION OF THE DRAWINGS

FIG. 1 shows functional annotation of genes on the mouse custom cDNA array. Percent of genes in each functional class are listed. Not shown in the figure is the 38% of the clones that are ESTs and 10% that were not annotated.

FIG. 2 demonstrates the reproducibility of the microarray. A sample was hybridized to itself in a dye-swap experiment. Only 0.065% of the spots had greater than a two-fold cy5/cy3 ratio; low intenstity signals were more variable. This hybridization also demonstrates that there was very minimal artifactual variation due to dye-bias.

FIG. 3A and FIG. 3B show functional annotation of rd1 at different time-points during degeneration compared to the entire array. FIG. 3A shows all differentially expressed genes treated as a group, and FIG. 3B divides the differentially expressed genes into genes up- and down-regulated genes.

FIG. 4 shows differential expression patterns divided into 8 clusters based on direction of change at different type points.

FIG. 5 shows genes which were differentially expressed at day 14. Duplicate genes at multiple time points were eliminated.

FIG. 6 shows genes which were differentially expressed at day 35. Duplicate genes at multiple time points were eliminated.

FIG. 7 shows genes which were differentially expressed at day 50. Duplicate genes at multiple time points were eliminated.

FIG. 8 shows genes at each of days 14, 35, and 50 which were differentially expressed without eliminating duplicates.

### DETAILED DESCRIPTION OF THE INVENTION

The inventors have developed a custom mouse retina microarray and used it in analyses of gene expression in degenerating rd1 mouse retina. The custom array was designed to expand the scope of possible analyses of normal and diseased retina by including genes known or predicted to be expressed in retinal neurons and glia. By comparing mutant and wild-type RNA profiles during rd1 hotoreceptor degeneration we identified genes and molecular pathways with altered regulation that are involved in neuronal cell death.

Furthermore, by comparing a time-series of degeneration we identified gene expression changes that may mediate disease progression, in particular, the non-cell autonomous death of cone photoreceptors. Finally, an additional finding in this study was the identification of over 150 retina-enriched genes, many of which map to known human disease loci and can be considered as reasonable candidate genes for retina diseases.

The methods of the present invention can be applied to any of the diseases of the retina, retinal pigment epithelium (RPE), and choroid. These include, but are not limited to, ocular neovascularization, ocular inflammation and retinal degenerations. Specific examples of these disease states include diabetic retinopathy, chronic glaucoma, retinal detachment, sickle cell retinopathy, senile macular degeneration, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy and lensectomy, vascular diseases retinal ischemia, choroidal vascular insufficiency, choroidal thrombosis, neovascularization of the optic nerve, diabetic macular edema, cystoid macular edema, retinitis pigmentosa, retinal vein occlusion, proliferative vitreoretinopathy, angioid streak, and retinal artery occlusion, and, neovascularization due to penetration of the eye or ocular injury. Additional relevant disease include the neuropathies, such as Leber's, idiopathic, drug-induced, optic, and ischemic neropathies.

Neurodegenerative disorders more broadly can also be treated and identified using the methods of the present invention. These include disorders of the central nervous system as well as disorders of the peripheral nervous system. Neurodegenerative disorders include, but are not limited to, brain injuries, cerebrovascular diseases and their consequences, Parkinson's disease, corticobasal degeneration, motor neuron disease (including ALS), multiple sclerosis, traumatic brain injury, stroke, post-stroke, post-traumatic brain injury, and small-vessel cerebrovascular disease. Dementias, such as Alzheimer's disease, vascular dementia, dementia with Lewy bodies, frontotemporal dementia and Parkinsonism linked to chromosome 17, frontotemporal dementias (including Pick's disease), progressive nuclear palsy, corticobasal degeneration, Huntington's disease, thalamic degeneration, Creutzfeld-Jakob dementia, HIV dementia, schizophrenia with dementia, and Korsakoff s psychosis, also are neurodegenerative disorders.

Neuronal cell death is a major feature of a variety of human neurological disorders, including the neurodegenerative diseases (such as Alzheimer's, Parkinson's, Huntington's and amyotrophic lateral sclerosis), stroke and trauma. Alzheimer's Disease afflicts about 4 million people in the United States, primarily the elderly. It is characterized by progressive memory loss, disorientation, depression and eventual loss of bodily functions.

Amyotrophic lateral sclerosis, afflicts about 30,000 Americans. It begins after age 40 and results in progressive weakness and paralysis. Huntington's Disease, which afflicts an estimated 25,000 patients in the United States, usually begins between the ages of 30 and 50 and includes violent, involuntary movements. The differentially expressed genes identified herein are applicable to these diseases as well.

Loss of neurons by a degenerative process is a major pathological feature of many human neurological disorders. Neuronal cell death can occur as a result of a variety of conditions including traumatic injury, ischemia, neurodegenerative diseases (e.g., Parkinson's disease, Huntington's disease, Alzheimer's disease, amyotrophic lateral sclerosis (ALS), stroke, or trauma), or as a normal part of tissue development and maintenance. Several inherited disorders produce late onset neuron loss, each of which is highly specific for particular neural cell types. The differentially expressed genes identified herein are applicable to these diseases as well.

Any type of neuronal cells can be used in the practice of the invention, for example, for screening for candicate drugs for treating neuronal cell death and disease resulting therefrom. Such cells include without limitation cells isolated from brain, neuroblastoma, astrocytoma, glioblastoma, medulloblastoma, retinoblastoma, and retina. Such cells can be isolated as is known in the art. Cell lines of these types are available from the American Type Culture Collection, Mannassas, Va. Cells that can differentiate into neurons, such as NT2, and PC12 cells can also be used to advantage.

Isolated and purified nucleic acids, according to the present invention are those which are not linked to those genes to which they are linked in the human genome. Moreover, isolated and purified nucleic acids are not present in a mixture, such as a library, containing a multitude of distinct sequences from distinct genes. They may be, however, linked to other genes such as vector sequences or sequences of other genes to which they are not naturally adjacent. The nucleic acids may represent either the sense or the anti-sense strand. Nucleic acids and proteins although disclosed herein with sequence particularity may be derived from a single individual. Allelic variants which occur in the population of humans are including within the scope of such nucleic acids and proteins. Those of skill in the art are well able to identify allelic variants as being the same gene or protein.

Isolated and purified proteins are not in a cell, and are separated from the normal cellular constituents, such as nucleic acids, lipids, etc. Typically the protein is purified to such an extent that it comprises the predominant species of protein in the composition, such as greater than 50, 60 70, 80, 90, or even 95% of the proteins present.

Using the proteins according to the invention, one of ordinary skill in the art can readily generate or obtain antibodies which specifically bind to the proteins. Such antibodies can be monoclonal or polyclonal. They can be chimeric, humanized, or totally human. Any functional fragment or derivative of an antibody can be used including Fab, Fab′, Fab2, Fab′2, and single chain variable regions. So long as the fragment or derivative retains specificity of binding for the endothelial marker protein it can be used. Antibodies can be tested for specificity of binding by comparing binding to appropriate antigen to binding to irrelevant antigen or antigen mixture under a given set of conditions. If the antibody binds to the appropriate antigen at least 2, 5, 7, and preferably 10 times more than to irrelevant antigen or antigen mixture then it is considered to be specific.

Techniques for making such partially to fully human antibodies are known in the art and any such techniques can be used. According to one such technique, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. See for example, Nina D. Russel, Jose R. F. Corvalan, Michael L. Gallo, C. Geoffrey Davis, Liise-Anne Pirofski. Production of Protective Human Antipneumococcal Antibodies by Transgenic Mice with Human Immunoglobulin Loci Infection and Immunity April 2000, p. 1820-1826; Michael L. Gallo, Vladimir E. Ivanov, Aya Jakobovits, and C. Geoffrey Davis. The human immunoglobulin loci introduced into mice: V (D) and J gene segment usage similar to that of adult humans European Journal of Immunology 30: 534-540, 2000; Larry L. Green. Antibody engineering via genetic engineering of the mouse: XenoMouse strains are a vehicle for the facile generation of therapeutic human monoclonal antibodies Journal of Immunological Methods 231 11-23, 1999; Yang X-D, Corvalan J R F, Wang P, Roy C M-N and Davis C G. Fully Human Anti-interleukin-8 Monoclonal Antibodies: Potential Therapeutics for the Treatment of Inflammatory Disease States. Journal of Leukocyte Biology Vol. 66, pp401-410 (1999); Yang X-D, Jia X-C, Corvalan J R F, Wang P, C G Davis and Jakobovits A. Eradication of Established Tumors by a Fully Human Monoclonal Antibody to the Epidermal Growth Factor Receptor without Concomitant Chemotherapy. Cancer Research Vol. 59, Number 6, pp1236-1243 (1999); Jakobovits A. Production and selection of antigen-specific fully human monoclonal antibodies from mice engineered with human Ig loci. Advanced Drug Delivery Reviews Vol. 31, pp: 33-42 (1998); Green L and Jakobovits A. Regulation of B cell development by variable gene complexity in mice reconstituted with human immunoglobulin yeast artificial chromosomes. J. Exp. Med. Vol. 188, Number 3, pp: 483-495 (1998); Jakobovits A. The long-awaited magic bullets: therapeutic human monoclonal antibodies from transgenic mice. Exp. Opin. Invest. Drugs Vol. 7(4), pp: 607-614 (1998); Tsuda H, Maynard-Currie K, Reid L, Yoshida T, Edamura K, Maeda N, Smithies O, Jakobovits A. Inactivation of Mouse HPRT locus by a 203-bp retrotransposon insertion and a 55-kb gene-targeted deletion: establishment of new HPRT-Deficient mouse embryonic sNM cell lines. Genomics Vol. 42, pp: 413-421 (1997); Sherman-Gold, R. Monoclonal Antibodies: The Evolution from '80s Magic Bullets To Mature, Mainstream Applications as Clinical Therapeutics. Genetic Engineering News Vol. 17, Number 14 (August 1997); Mendez M, Green L, Corvalan J, Jia X-C, Maynard-Currie C, Yang X-d, Gallo M, Louie D, Lee D, Erickson K, Luna J, Roy C, Abderrahim H, Kirschenbaum F, Noguchi M, Smith D, Fukushima A, Hales J, Finer M, Davis C, Zsebo K, Jakobovits A. Functional transplant of megabase human immunoglobulin loci recapitulates human antibody response in mice. Nature Genetics Vol. 15, pp: 146-156 (1997); Jakobovits A. Mice engineered with human immunoglobulin YACs: A new technology for production of fully human antibodies for autoimmunity therapy. Weir's Handbook of Experimental Immunology, The Integrated Immune SysNM Vol. IV, pp: 194.1-194.7 (1996); Jakobovits A. Production of fully human antibodies by transgenic mice. Current Opinion in Biotechnology Vol. 6, No. 5, pp: 561-566 (1995); Mendez M, Abderrahim H, Noguchi M, David N, Hardy M, Green L, Tsuda H, Yoast S, Maynard-Currie C, Garza D, Gemmill R, Jakobovits A, Klapholz S. Analysis of the structural integrity of YACs comprising human immunoglobulin genes in yeast and in embryonic sNM cells. Genomics Vol. 26, pp: 294-307 (1995); Jakobovits A. YAC Vectors: Humanizing the mouse genome. Current Biology Vol. 4, No. 8, pp: 761-763 (1994); Arbones M, Ord D, Ley K, Ratech H, Maynard-Curry K, Otten G, Capon D, Tedder T. Lymphocyte homing and leukocyte rolling and migration are impaired in L-selectin-deficient mice. Immunity Vol. 1, No. 4, pp: 247-260 (1994); Green L, Hardy M, Maynard-Curry K, Tsuda H, Louie D, Mendez M, Abderrahim H, Noguchi M, Smith D, Zeng Y, et. al. Antigen-specific human monoclonal antibodies from mice engineered with human Ig heavy and light chain YACs. Nature Genetics Vol. 7, No. 1, pp: 13-21 (1994); Jakobovits A, Moore A, Green L, Vergara G, Maynard-Curry K, Austin H, Klapholz S. Germ-line transmission and expression of a human-derived yeast artificial chromosome. Nature Vol. 362, No. 6417, pp: 255-258 (1993); Jakobovits A, Vergara G, Kennedy J, Hales J, McGuinness R, Casentini-Borocz D, Brenner D, Otten G. Analysis of homozygous mutant chimeric mice: deletion of the immunoglobulin heavy-chain joining region blocks B-cell development and antibody production. Proceedings of the National Academy of Sciences USA Vol. 90, No. 6, pp: 2551-2555 (1993); Kucherlapati et al., U.S. Pat. No. 6,1075,181.

Antibodies can also be made using phage display techniques. Such techniques can be used to isolate an initial antibody or to generate variants with altered specificity or avidity characteristics. Single chain Fv can also be used as is convenient. They can be made from vaccinated transgenic mice, if desired. Antibodies can be produced in cell culture, in phage, or in various animals, including but not limited to cows, rabbits, goats, mice, rats, hamsters, guinea pigs, sheep, dogs, cats, monkeys, chimpanzees, apes.

Antibodies can be labeled with a detectable moiety such as a radioactive atom, a chromophore, a fluorophore, or the like. Such labeled antibodies can be used for diagnostic techniques, either in vivo, or in an isolated test sample. Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing antitumor agents (e.g., antisense oligonucleotides, plasmids which encode for toxins, methotrexate, etc.); and other antibodies or antibody fragments, such as F(ab).

Those of skill in the art will readily understand and be able to make such antibody derivatives, as they are well known in the art. The antibodies may be cytotoxic on their own, or they may be used to deliver cytotoxic agents to particular locations in the body.

The antibodies can be administered to individuals in need thereof as a form of passive immunization.

Drugs can be screened for the ability to modulate expression of the genes, mRNA, and protein which are identified herein. Cell populations can be contacted with test substances and the expression of neuronal cell death markers determined. Test substances which decrease the expression of up-regulated neuronal cell death markers are candidates for inhibiting neuronal cell death. Conversely, test substances which increase the expression of down-regulated neuronal cell death markers can be identified as candidate drugs for causing neuronal cell death. In cases where a biological or enzymatic activity of a NM is known, agents can be screened for their ability to decrease or increase the activity or amount of activity present in a cell.

Expression can be monitored according to any convenient method. Protein or mRNA can be monitored. Any technique known in the art for monitoring specific genes' expression can be used, including but not limited to ELISAs, SAGE, custom or commercial microarray hybridization, Western blots. Changes in expression of a single marker may be used as a criterion for significant effect as a potential pro-neuronal cell death or anti-cell death agent. However, it also may be desirable to screen for test substances which are able to modulate the expression of groups of such markers, such as modulators of at least 5, 10, 15, or 20 of the relevant markers. Inhibition of NM protein activity can also be used as a drug screen.

Neuronal cell death markers identified herein were identified using available reagents for probes. In some cases these probes are human. In other case they derive from other mammalian species. Each gene has an ortholog in humans, and the human ortholog is to be used for treating humans. When cells, cell lines, and whole animal models of other species are used, it is preferred that the species-appropriate ortholog be used. For example, mouse counterparts to human NMs can be used in mouse models or in cell lines or in vitro to evaluate potential anti-neuronal cell death or pro-neuronal cell death compounds or therapies. Their expression can be monitored as an indication of effect.

Nonetheless, as demonstrated in the examples below, probes for orthologs of other species can be used.

Test substances for screening can come from any source. They can be from libraries of natural products, combinatorial chemical libraries, biological products made by recombinant libraries, etc. The source of the test substances is not critical to the invention. The present invention provides means for screening compounds and compositions which may previously have been overlooked in other screening schemes.

Nucleic acids and the corresponding encoded proteins of the markers of the present invention can be used therapeutically in a variety of modes. The nucleic acids and encoded proteins can be administered by any means known in the art. Such methods include, using liposomes, nanospheres, viral vectors, non-viral vectors comprising polycations, etc. Suitable viral vectors include adenovirus, retroviruses, and sindbis virus. Administration modes can be any known in the art, including parenteral, intravenous, intramuscular, intraperitoneal, topical, intranasal, intrarectal, intrabronchial, etc. Such administrations can be used to reduce or eliminate cell death (down-regulated genes or proteins) or induce cell death (up-regulated genes or proteins). The pathological condition of the patient will determine which type of gene or protein should be used.

Specific biological antagonists of NMs can also be used to therapeutic benefit. For example, antibodies, T cells specific for an NM, antisense to an NM, and ribozymes specific for an NM can be used to restrict, inhibit, reduce, and/or diminish neuronal cell death (up-regulated genes or proteins). Conversely, antagonists of down-regulated genes or proteins can be used to induce or stimulated neuronal cell death. Such antagonists can be administered as is known in the art for these classes of antagonists generally.

Mutations in genes expressed in rod photoreceptors frequently lead to secondary degeneration of cone photoreceptors in human retinitis pigmentosa (RP). It is the death of cones that cause profound vision loss. To gain insight into the pathogenesis of RP we have studied molecular pathways contributing to rod and cone death in rd1 mutant mice. Although rd1 photoreceptor degeneration is far more rapid than any human disease, these mice offer the benefit of investigating molecular mechanisms originating from a defined primary mutation that correlate with a particular histological pattern of cell death. Specific time-points during degeneration were analyzed using a custom cDNA microarray to define genes that are differentially expressed in the retina under conditions where the rods are present or absent. Groups of genes were identified that may play roles in mediating rod photoreceptor degeneration and in the initiation and progression of cone photoreceptor death.

Two methods of assigning physiological significance to the differential expression were employed: statistically, based on clustering the time-dependent expression changes, and biologically, based on functional annotation. Combining the two methods leads to several testable hypotheses that implicate specific genes and pathways in rd1 rod and cone photoreceptor degeneration.

A large group of genes showed increased expression following rod degeneration and remained high during cone degeneration. This category featured genes known to be involved in growth and differentiation, such as the Wnt pathway gene dickkopf 3, membrane glycoprotein M6, insulin-like growth factor binding protein 5 and Bin-1 (also known as myc box dependent interacting protein 1). The appearance of these genes may represent cellular pathways that mediate changes in the tissue architecture in degenerating rd1 retinas, could reflect the hypothesized reentry into the cell cycle of dying cells, or could be related to anti- and pro-apoptotic functions for some of these genes. For example, dickkopf 3 activity in the retina has not been explored but it has been reported to be down-regulated in lung carcinoma, suggesting a role in cellular proliferation. Increased expression of the Wnt-related genes SFRP and frizzled-4 have been identified in human retina degenerations, and Wnt is known to regulate apoptosis in vitro. Membrane glycoprotein M6 was associated with calcium-mediated neuronal differentiation of PC12 cells (Mukobata 2002), and insulin-like growth factor binding proteins are known to mediate neuronal differentiation. The increased expression of the genes described above is consistent with a well-described class of growth factors, the neurotropins, which can protect dying rod photoreceptors in several damage paradigms, such as GDNF in rd1 mice (Frasson, 1999).

It has been hypothesized that the protective effect of neurotrophic factors on photoreceptors occurs indirectly through Muller cells (Harada 2000; others). Although there are reports of increased GFAP (Ekstrom 1998) and immediate-early genes in Muller cells during rod degeneration (Rich, 1987), the role of retina glia cells in cone photoreceptor degeneration in rd1 mice is unclear. Based on the activity of glia in other degeneration conditions, such as light damage (Harada 2002), it is plausible that they may have a function in protecting or promoting degeneration in rd1. The reactive changes in retina glia involve cell shape changes, increased GFAP, increased proliferation capacity, redistribution. Genes that had increased expression on our arrays, particularly genes involved in growth and differentiation, could possibly be part of the glial response. Vimentin, an intermediate filament protein that is associated with Muller cell hypertrophy (Lewis 1995), was increased at P35 and P50. Several immune-related genes, such as the lymphocyte antigen 6 complex, also showed increased expression, consistent with activation of microglia. Interestingly, the pleiotrophin gene, which is found in glia in the brain and is involved in communication with neurons (Silos-Santiago, 1996), was increased at P50. Therefore, it appears possible that the cone degeneration that follows loss of rods may be mediated by intercellular communication involving glia cells.

However, detailed cell localization of the genes will be necessary to demonstrate if glia cells express these genes, and which type of glia, Muller glia, microglia or astrocytes.

Genes that had higher increases in expression in mutant retinas at P35 than the other time-points tested coincide with the early stages of cone degeneration. One example is crystallin beta A4, which was increased 4-fold in mutant P35 retinas, compared with age-matched wildtype. We also observe increased crystallin beta A4 during rod degeneration at P14. In contrast, there was more than a two-fold decreased expression of crystallin beta A4 and another crystallin, alpha 1, at P50. Crystallins are the major structural proteins in the lens, but are increasingly being recognized to be present in other tissues {Bhat, 1989} including retina (Magabo 2000). Several studies have shown that alpha- and beta-crystallins have chaperone activity. Furthermore, both types of crystallins are phosphorylated through cAMP-dependent and -independent pathways (Kantorow, 1997) suggesting a possible signalling function. Although beta-crystallins have not be previously reported in degenerating retina, Jones et al (1998) reported an increase in expression of a-crystallin at P18 in rd1 retinas, a time-point after most rods have died, and did not detect an increase during the peak of rod degeneration (tested at P15). Time-points later than P18 were not analyzed. The increased expression was shown to be in the inner retina, and was suggested to be in ganglion cells and Muller end processes. Interestingly, alpha-crystallins have been demonstrated to inhibit oxidative stress induced apoptosis in RPE cells in culture (Alge 2002). In a lens epithelial cell line, a-crystallin was shown to prevent apoptosis by inhibiting caspase-3 activation (Mao 2001). Furthermore, oxidative stress-induced apoptosis in these cells was associated by decreased a-crystallin (Mao 2001). Therefore, it is possible that non-refractive properties of crystallins are involved in protecting cones from degeneration, possibly through Muller cell-cone cell interactions, through a balance of increased and decreased crystallins.

During cone degeneration at P35 and P50 genes known to be involved in oxidative stress response had increased expression, at a frequency higher than the proportionate amount of this class of genes on the array. For example, antioxidant protein 2, the intermediate filament vimentin, glutathione peroxidase 2, and paraoxonase all have reported roles in stress responses. Oxidative injury has been proposed to contribute to the pathogenesis of AMD, and to be involved in cell death in axotomized ganglion cells and light damaged photoreceptors. In this case, oxidative injury from hyperoxia in rd1 could arise from increased physical proximity of the cone photoreceptors to the choroid due to loss of rods.

Gene expression data obtained from all array projects are obviously limited by the identity of the genes on the arrays. In our study, the arrays were designed to be more representative of retina than many commercial arrays, although it is by no means a complete sampling of all retina genes. The arrays will be continuously supplemented with additional genes, particularly including genes that were not initially available from the libraries used. Since cDNA arrays may not properly differentiate between some splice forms or gene family members, it is possible that a closely related gene may actually have been measured. Further investigation into any of the genes identified here should also include analysis of genes with similar sequence. In this study we used whole retina tissue to address non-cell autonomous expression changes. Future work will take advantage of single cell isolation to address cell-specific changes and to clone low-abundance genes that may be diluted out when sampling the whole tissue.

It is interesting that a different complement of genes is increased during rod death than cone death. This work was focused on exploring cone degeneration, and earlier time-points are necessary to explore gene expression changes involved in initiating rod degeneration. At this point it is impossible to distinguish between primary and secondary changes. However, secondary changes should not be assumed to be benign as they may directly affect the progression of degeneration. In summary, we have identified gene expression changes that are correlated to critical physiological events during retinal degeneration. Future work will explore the precise function of these genes in rd1 retina degeneration. Additionally, a side-line of this study validated the use of the custom murine array as a gene discovery tool. Further analysis on the genes that were mapped to retina disease loci will be used to determine whether these candidate genes are involved in retina disease or can be used as molecular markers of pathogenesis.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

Custom Retina Array Preparation

The retina microarray contains 5376 genes and was assembled from two sources, individual purchased clones and selected clones from a mouse eye cDNA library. The purchased clones were chosen by searching OMIM, PubMed and Unigene EST databases at NCBI {Altschul 1990} for all genes with roles in normal retinal function, development and degeneration, and genes implicated in retinal diseases. In order to include genes that may not have been selected from the database searchers, we also included all murine ESTs expressed in retina and brain that were available in Unigene. The selected clones (3752 unique sequences) were purchased from Research Genetics (Huntsville, Ala.). An additional 1624 clones were derived from a fully sequenced adult mouse eye cDNA library that was generously provided by Drs. Jeremy Nathans and Amir Rattner (Johns Hopkins University). The identities of the clones in the cDNA library were determined by BLAST searches in the NCBI public database {Altschul 1990}, and duplicate clones were eliminated from the array by comparing corresponding Unigene numbers. Clones that gave poor sequence and did not have matches in the database were resequenced to determine their identity. The clone lists were filtered to eliminate duplicates in order to produce a non-redundant clone set. Functional annotation was performed using the SOURCE and GO databases, as well as manual annotation from NCBI PubMed and LocusLink databases.

The clones were rearrayed into 96-well plates and grown overnight in LB/10% glycerol (for bacterial clones) or SM buffer (for phage clones). The clone inserts were PCR amplified from the bacterial or phage stocks in duplicate 100 ul reactions, using primers from flanking vector sequences. The PCR products were purified using Millipore Multiscreen PCR filters and eluted in TE (10 mM Tris-HCl pH 7.5, 0.1 mM EDTA) or water. Amplification efficiency and purification of each clone was verified by analyzing an aliquot of purified product on a 2% agarose gel. The PCR products were suspended in a final concentration of 50% DMSO and arrayed in duplicate onto SuperAmine silylated slides (Telechem, Sunnyvale, Calif.) using the Microgrid II arrayer (Biorobotics) with 100 micron-tip quill pins (Biorobotics, Cambridge, UK). Each gene was printed in duplicate on each array, separated by half the length of the slide, and was considered separately in the analyses. The printing environment was maintained at approximately x% humidity and x C. Spot size, morphology and quality was verified by sybr-green II staining (Molecular Probes) on several slides from each print run prior to use.

### EXAMPLE 2

RNA Isolation and Probe Labeling

All procedures involving mice were carried out in accordance with the statement by the Association for Research in Vision and Ophthalmology for the Use of Animals in Ophthalmic and Vision Research and was approved by the Animal Care and Use Committee at The Johns Hopkins University. Tissue samples were obtained from homozygous rd1/rd1 and age-matched control C57BL/6 mice and processed immediately or flash-frozen and stored at −80 ° C. Retinas were dissected under a microscope to exclude pigmented epithelium and other extraretinal tissue. Total RNA was isolated using phenol-chloroform extraction (Trizol reagent, Invitrogen). Retinas from four animals were pooled for the retina extractions due to the small size of the retina tissue and low RNA yield per retina and also to minimize the effect of biological variability. A reference sample was also made, composed of 30% retina RNA and 70% brain RNA, derived from a mix of strains at various ages, from juvenile to adult. RNA integrity was assessed by gel electrophoresis, A₂₆₀/A₂₈₀ absorbance ratios and by analyzing an aliquot on the Bioanalyzer (Agilent).

### EXAMPLE 3

Hybridization and Data Analysis

Probe preparation and hybridization were based on protocols described previously {Hegde 2000}. A minimum of four replicates was performed for each experiment, and “dye-swaps” were used to minimize the potential for differential dye-effects. The RNA used in replicate experiments for the mutant mice was from different preparations. A reference sample comparison (wildtype versus reference and mutant versus reference) was used for time-points post-natal day (P) 14 and P50 and a direct comparison (wildtype vs mutant) was used at time-point P35. Twenty micrograms of total RNA were treated with 2 U amplification grade DNase I (Ambion) and purified using RNeasy columns (Qiagen). Eluted RNA, free of small RNA species and genomic DNA fragments, was labeled using an indirect dye-incorporation method. Briefly, first-strand cDNA was synthesized from 20 ug total RNA using SuperScript II reverse transcriptase (Invitrogen) with 6 ug random hexamers, 10 mM DTT and aminoallyl dUTP/dNTP solution (final concentrations: 1.25 mM dATP, dCTP and dGTP, 1 mM dTTP, 0.25 mM aminoallyl dUTP (Sigma)), at 42 C for 16 hr. The RNA template was then hydrolyzed with 0.1 M sodium hydroxide, and unincorporated nucleotides were removed using QIAquick PCR purification kit (Qiagen) with Tris-free phosphate wash buffer (4.75 mM K₂HPO₄, 0.25 mM KH₂PO₄, pH 8.0, 80% ethanol) and elution (3.80 mM K₂HPO₄, 0.2 mM KH₂PO₄, pH 8.5) buffer. Eluted DNA was dried under vacuum and resuspended in 4.5 ul 0.1 Na2CO3 buffer, pH 9.0, mixed with an equal volume of monoreactive NHS-cy3 or -cy5 dye (Amersham) resuspended in DMSO then incubated for 2 hr at room temperature. The dye-coupling was terminated with 100 mM sodium acetate and free dye was removed using the QIAquick PCR purification columns. Total pmol of dye incorporation and dye molecules per nucleotide were calculated using A₅₅₀ (for cy3) and A₆₅₀ (for cy5) readings.

The aminoallyl labeled cDNA probes were dried and resuspended in hybridization solution (50% formamide, 5×SSC, 0.1% SDS). The printed slides were UV cross-linked and prehybridized in 5×SSC, 0.1% SDS, 1% BSA for at 42 C for 45 minutes immediately prior to hybridization. Repetitive sequences were blocked in the probe by incubating the probe solution with 10 ug polyA DNA (Pharmacia) and 10 ug Cₒt fraction mouse DNA (Invitrogen), the probe was denatured at 95° C., cooled on ice for 1 minute then added to a prehybridized slide, covered with a washed glass coverslip and incubated at 42° C. overnight, in a vacuum-sealed hybridization chamber (GeneMachine). The hybridized arrays were washed at 42° C. for four minutes in 1×SSC/0.2% SDS, followed by two four minute room temperature washes, in 0.1×SSC/0.2%SDS then 0.1×SSC. The slides were dried with an air can and fluorescent images of the hybridized microarrays were generated by a Scan Array 5000 scanner and Scan Array software (Perkin Elmer).

Spot-finding and image analysis and quantification was performed on the scanned microarray data TIFF files using Imagene software (Biodiscovery, Inc., Marina del Rey, Calif.). Since the quality of low intensity spots are not as good as others, usually the low intensity spots are not included in the analysis. However, because the proportion of low intensity spots is not ignorable in our data, and some of the genes with potential interest are expected to be expressed at low level, we don't want to simply discard those spots in the analysis. We treated the low intensity spots within each array, the low intensity spots flagged by software are floored to a threshold, which is defined by the average intensity of all low intensity spots within the same array. This means that the flagged spots with intensity lower than the threshold will be upgraded to the threshold, while spots with intensity higher than the threshold will remain the original values. This floor method maximized the proportion of informative spots included for later analysis, and at the same time minimized the effect of poor quality data. If a spot has both cy3 and cy5 channels floored, it will not be included in the normalization process. The final log ratio of this spot will be assigned to be zero. For all other spots, we first did loess normalization based on MVA plot. Then we checked the plate effect and pin effect on each array based on box plot. Some plots showed clear decreasing or increasing pattern. Some plots showed within an array, certain plate/pin diverged from the overall distribution of the whole array. For arrays with plate/pin effect, we applied median normalization within each plate/pin, the median log ratio is subtracted from the loess normalized log ratio. Whether plate/pin normalization is necessary is decided slide by slide. Not all arrays have the plate/pin normalization. {Chowers et al, in press}. The image data was normalized using the Genesight software program (Biodiscovery, Inc., Marina del Rey, Calif.) in order to compare across replicate slides, using the mean of all spots on the slide for each fluorescent channel. Local background was subtracted from the spot signals and the data were log₂ transformed. Duplicate spots were printed on each array but they were considered separately in all analyses since they are not completely independent. Statistical analysis of the transformed data was performed using the Significance Analysis of Microarrays (SAM) program created by Tusher et al {Tusher, 2001}.

A clustering method was developed for the analysis due to the fact that our experiments included a limited number of time points and experimental conditions. Statistical t-test scores were calculated between all successive time points as follows: Suppose there are N time points in the experiment, we will calculate t-test scores for N−1 time steps. Considering two successive time points, we defined expression changes of “up”, “down”, or “no change” by choosing a set of threshold for t-test scores. With combinations of different expression changes at each time step, all possible expression patterns can be constructed. For example, for an experiment with 3 time points, we would have 9 different patterns (“up-up”, “up-down”, “no change-up”, and so on). Permutation was performed to evaluate the statistical significance for each pattern. For each expression pattern, we counted the numbers of genes that have satisfied the criteria for the pattern before and after the permutation. The ratio between these two numbers is also called false discovery rate.

### EXAMPLE 4

Quantitative PCR

cDNA was synthesized from 1 ug of total RNA using Thermoscript reverse transcriptase (Invitrogen). Quantitative real-time PCR (QPCR) was then performed using the Lightcycler-FastStart DNA Master SYBR Green I kit (Roche), according to the manufacturer's instructions. Each QPCR assay reaction also included amplification of control genes actin or ARP (acidic ribosomal phosphoprotein P0 (ARP)) {Simpson, 2000} from each cDNA reaction. Primers were chosen from exons separated by large introns, and the PCR reaction quality and specificity was verified by melting curve dissociation analysis and gel electrophoresis of the amplified product. Primer sequences were: ARP-sense 5′-ATCTGCTGCATCTGCTTG-3′ (SEQ ID NO: 1); ARP-antisense 5′-CGACCTGGAAGTCCAACTAC-3′(SEQ ID NO: 2); CACNG4 (calcium channel g4)-sense 5′-ATTACGACCACGACAGCTC-3′(SEQ ID NO: )3; CACNG4-antisense 5′-TTCGTCACGTTTGTCACTG-3′(SEQ ID NO: 4); anti-oxidant protein 2-sense 5′-AGCTGACAGGCACAAAGC-3′(SEQ ID NO: 5); anti-oxidant protein 2-antisense 5′-CAGTAAAGAATCCCGAGA-3′(SEQ ID NO: 6); vimentin-sense 5′-GAAACTGCACGATGAAGAG-3′(SEQ ID NO: 7); vimentin-antisense 5′-TAGGTGGCGATCTCAATGTC-3′(SEQ ID NO: 8); IMAGE 4505626 sense 5′-ATGGCAGGAGCATGAAATG-3′(SEQ ID NO: 9); IMAGE 4505626 antisense 5′-TAGTAGTGGTGATCATGGTG-3′(SEQ ID NO: 10); dickkopf 3-sense 5′-AGACAGTCATTACATCTGTAG-3′(SEQ ID NO: 11); dickkopf3-antisense 5′-GTGATGAGATCCAGCAGCT-3′(SEQ ID NO: 12); Mm.156168-sense 5′-AATGACAGGTGGCTTGAAC-3′(SEQ ID NO: 13); Mm. 156168-antisense 5′-GTGTATAAGCGATACTACGA-3′(SEQ ID NO: 14); CRX sense 5′-CAGGGTTCAGGTTTGGTTC-3′(SEQ ID NO: 15); CRX antisense 5′-CATCTGTGGAGGGTCTTGG-3′(SEQ ID NO: 16). For quantification, a standard curve was generated from at least three two-fold serial dilutions of the cDNA template using. Relative transcript levels of each gene were calculated from the mean of duplicate cDNA dilutions using the second derivative maximum values from the linear regression of cycle number versus log concentration of the amplified gene. Amplification of the control genes was used for normalization.

### EXAMPLE 5

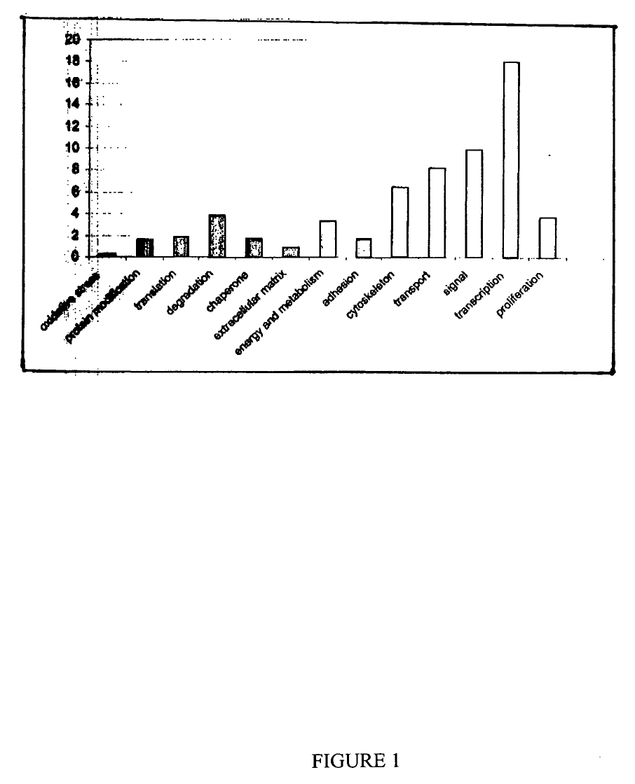

To generate the mouse retina custom array, we were interested in genes that would be likely candidates for involvement in normal and disease processes in the retina. Genes were chosen that belonged to either of two categories: function or tissue distribution. The first group included genes involved in essential cellular mechanisms such as transcription and signaling, genes that had known activities in the retina, such as phototransduction and outer segment structural proteins, and genes implicated directly in disease by identified mutations, or indirectly, as part of general disease processes of retinopathies and neurodegeneration, such as apoptosis and angiogenesis. The genes were selected by searching literature and on-line database resources (NCBI, PubMed, OMIM) (see above). Additionally, we wanted to include all genes expressed in the retina regardless of whether their function was known by selecting all available mouse retina ESTs. Because a limited number of mouse retina libraries were in Unigene at the start of the project, we also selected all genes and ESTs expressed in the brain in order to have a large representation of neuronal and glial genes on the array. Additionally, we included approximately 1,600 clones from a fully sequenced adult mouse eye cDNA library (Drs. J. Nathans and A. Rattner, The Johns Hopkins University). In total, we obtained 5,376 clones for the murine retina non-redundant gene set. The genes were annotated into the classes shown in FIG. 1. Over a third of the genes in the set included ESTs, predicted genes and genes of unknown function.

Numerous optimization and validation studies for probe labeling and hybridization were performed with the printed arrays. For example, suspension of the PCR products in 50% DMSO was shown to produce better spot morphologies and hybridization intensities than SSC-containing buffers (3×SSC or 25%DMSO/3×SSC) (data not shown). It was also determined that aminosilane-coated slides produced more consistent results than polylysine-coating, and various aminoallyl-UTP to TTP ratios were tested to determine which gave the most favorable dye incorporations and signal-to-noise ratios. All subsequent analyses were performed with the optimized protocol described in Materials and Methods.

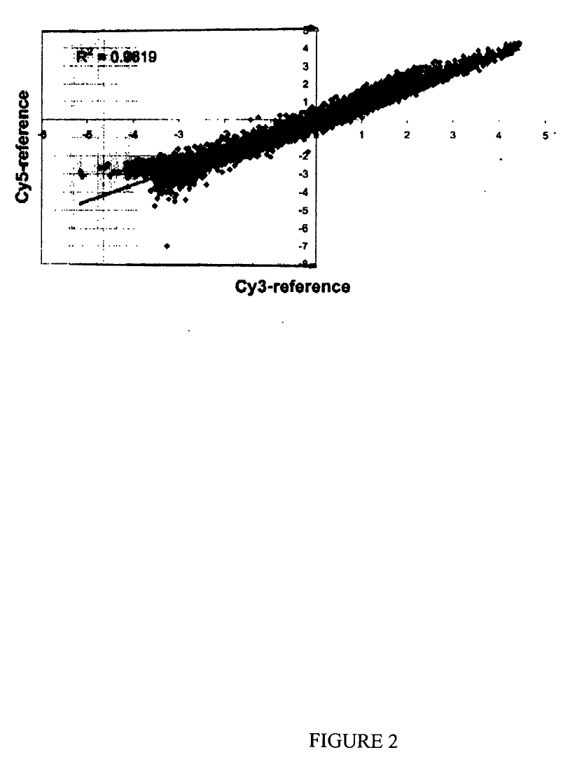

To verify microarray reproducibility we compared identical samples using a self-self hybridization. As shown in the scatterplot in FIG. 2, this hybridization resulted in a high correlation (R²=0.9619). Only 7 genes out of 10752 spots on the slide (each gene printed twice) (0.065%) had greater than a two-fold cy5/cy3 ratio. As expected, low intensity signals were more variable: all the genes that had spurious differential expression were close to background intensity levels. This test also demonstrates that there was very minimal artifactual variation from dye-effect. However, as typically observed in microarray experiments, there was variability in the absolute signal intensities and calculated ratios among the arrays. To compensate for this measurement variability, we determined differential expression using a statistical significance analysis test (see below).

### EXAMPLE 6

Identification of Genes Differentially Expressed During Retina Degeneration in the Mouse

A time-series experiment was performed by comparing the gene expression profiles of normal and degenerating homozygous rd1 retina at P14 retinas (corresponding to the peak of rod degeneration), P35 (at post-rod and early cone degeneration) and P50 (during cone degeneration). By comparing wild-type and mutant retinas this analysis would identify gene expression changes that may contribute to pathogenesis. Also, by comparing different time points during the course of the disease, we would identify genes that may promote progression of degeneration, particularly cone death.

To identify genes altered in the rd1 mice, we used the statistical test Significance Analysis of Microarrays (SAM) {Tusher 2001} to distinguish real gene expression changes from those identified due to chance (see Methods for normalization and statistical protocols used). This statistical tool differentiates true expression differences from changes due to random fluctuations, permitting identification of small, yet potentially biologically significant, expression changes. A false discovery rate (FDR) is calculated by considering the variability in experimental measurements and comparing the expression ratios from the microarrays to ratios calculated by randomly permutating the control and experimental groups. Therefore, gene expression changes were considered significantly different between wildtype and mutant if they were identified in the SAM analysis using a low FDR. Approximately 3% of genes on the array were differentially expressed at least two-fold at each time-point. This small number of genes being changed indicates that our analyses were appropriately stringent and is consistent with other studies using either oligonucleotide or cDNA arrays in which only a fraction of total genes have significant alterations in expression. At P14, with an FDR of 16%, 183 genes were differentially expressed, 63 increased and 120 decreased. At 0.2% FDR at P35 there were 36 genes increased and 182 decreased, and at P50 (FDR 0.5%), 62 increased and 146 decreased genes (supplemental Table 1). As expected, most of the down-regulated genes (16%, 18% and 20% at P14, P35 and P50, respectively) include those known to be expressed in rod photoreceptors, such as opsin and phosducin. Mutations in many of these genes have been identified in human hereditary retinal degenerations. Later time-points had greater decreases in these genes. Interestingly, many genes in this class included ESTs and genes of unknown or uncertain function.

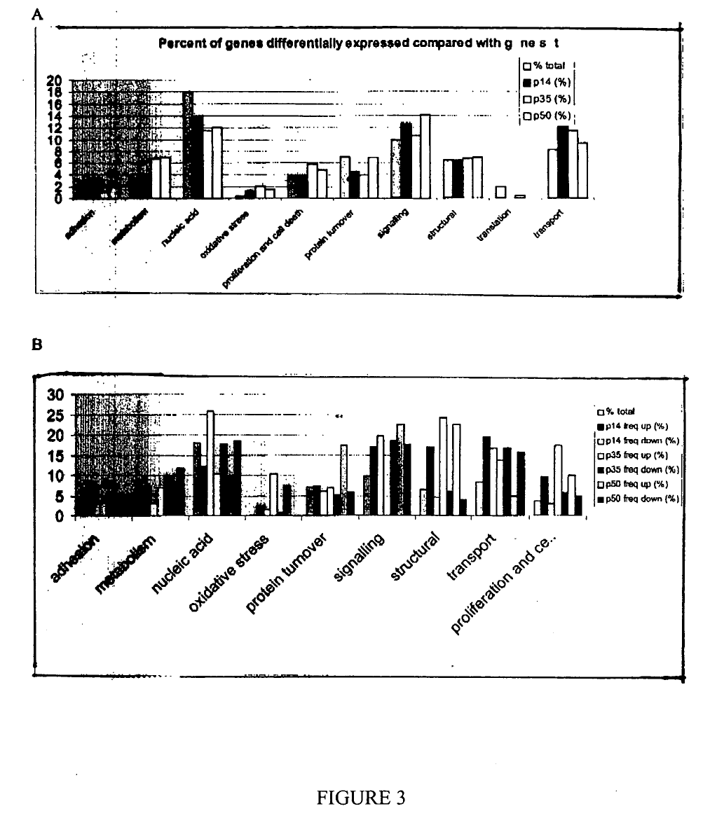

The functional annotation of the differentially expressed genes is shown in Table 1 and FIG. 3. At P14, during rod degeneration, the most highly represented functional classes were genes involved in signaling and transport (showing increased and decreased expression). Genes involved in the cytoskeleton/structure showed mostly increased expression, whereas genes involved in nucleic acid metabolism had decreased expression. At P35, the same functional groups were represented, but signaling genes only showed decreased expression. Additionally, genes involved in oxidative stress and growth and proliferation were observed, and these had predominantly increased expression. At P50, more genes involved in protein modification/degradation were increased than at the other two time-points. Also, fewer transport genes were increased compared with the other two time-points. Signaling, nuclei acid metabolism and structural genes were also represented. Interestingly, although the differentially expressed genes included those known to be involved in cell death in vitro or in vivo, such as GFAP and vimentin (see Discussion), many of the genes we identified had not previously implicated in retina degeneration in the mouse.

Comparing across the three time-points showed that there was little overlap in the genes with increased expression (many decreased genes are shared since they were likely expressed in rod photoreceptors). The difference in regulated genes suggests the involvement of distinct molecular pathways in rod versus cone death. Thus, the array results can distinguish rod degeneration from cone degeneration: 43 genes that are up-regulated during cone degeneration at P50 are not increased during rod degeneration at p14. The most commonly represented genes that are up-regulated during at P50 and differ from genes seen at P14 are involved in cytoskeleton and adhesion function. Specific genes involved in intercellular signaling and protein modification were increased at P50 and not P14. Interestingly, several calcium-activated proteins are upregulated at P50 (eg. calpain, calmodulin). In contrast, genes involved in cellular transport were more highly represented during rod degeneration and not cone degeneration, particularly those involved in modulating ion transport and neuronal transmission (e.g., Ly6, NMDA1 subunit zeta 1).

We next compared the differential expression annotation to the frequencies of the functional classes of the entire array (FIG. 3). This comparison further illustrates the over-representation of certain gene classes at each time-point compared with their frequency in the gene set. Of note is the large representation of genes involved in signaling processes at P14 and P50 compared with the whole array, transport genes at P14 and P35, and metabolism genes at P35 and P50. Also, genes involved in oxidative stress were more prominent during degeneration at P35 (1.9% vs 0.2% in the entire gene set). In contrast, there was very low representation at any time-point of genes involved in translation mechanisms compared with the whole array.

A clustering method was used to infer biological information from the gene expression data by dividing the large data set of gene expression changes into smaller groups to identify genes that are behaving similarly. Standard clustering methods, such as hierachical clustering, self-organizing map (SOM), or k-mean clustering, are based on the distances between pairwise gene expression profiles (using correlation coefficients). This is problematic if only a few time points or experiment conditions being compared, such as the three time-point wild-type/mutant design used in this study. Therefore, a clustering method was developed using t-test statistics, which allowed us to determine genes that clustered based on a statistical significant threshold (see above). Similar to the SAM analysis described above, the time-series t-test clustering method also permitted consideration of measurement variability and allowed calculation of a false discovery rate.

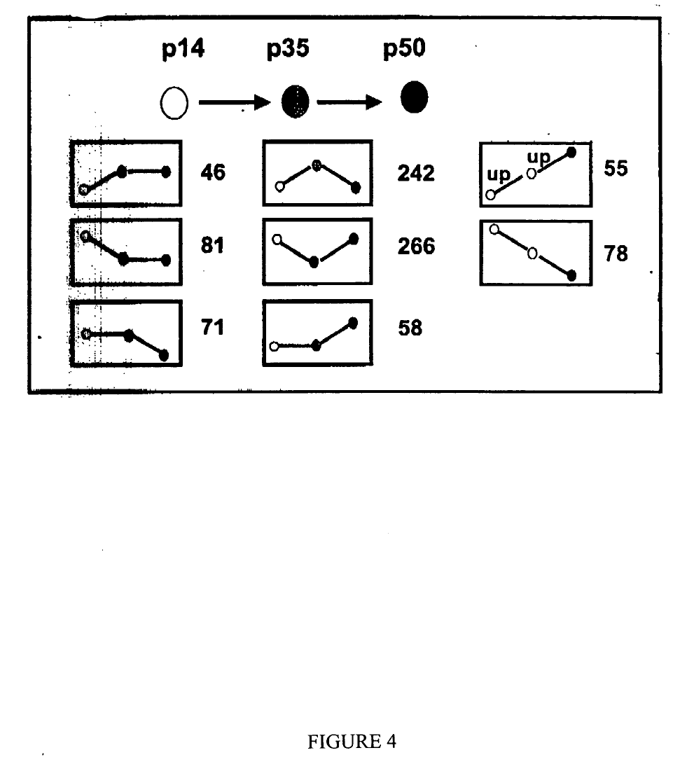

The clustering analysis demonstrated that temporal expression pattern varies among the genes: some genes show continuous increases, others decrease, and others have time-point specific expression changes. Nine different clusters were identified (see FIG. 4, Table 2), based on the difference in expression between P35 and P14 (t2−t1), and P50 and P35 (t3−t2). To assess of the predictability of this clustering method we determined which cluster contained known retinal genes. Indeed, many of the genes in cluster 7 (“down-down”) and 4 (“down-no change”) are known retina-enriched genes. Interestingly, the largest number of genes (786 genes) was grouped into cluster 5 (“down-up”), which contained genes that decreased in the interval following rod degeneration at P35 relative to p14 and then increased during cone degeneration at P50 relative to P35. Since the clustering method relates differential expression between mutant and wildtype using the mutant/wildtype ratios to differential expression at each time-point, the down-up cluster does contain genes that had reduced expression in the mutant compared to wildtype, but just not to the same extent as in the previous time-point. The second largest group, cluster 2 containing 737 genes, was the opposite pattern (“up-down”), showing increased expression after rod degeneration at P35 and decreased expression during cone degeneration at P50. The coordinated expression changes suggest that these clustered genes may participate in similar biological processes.

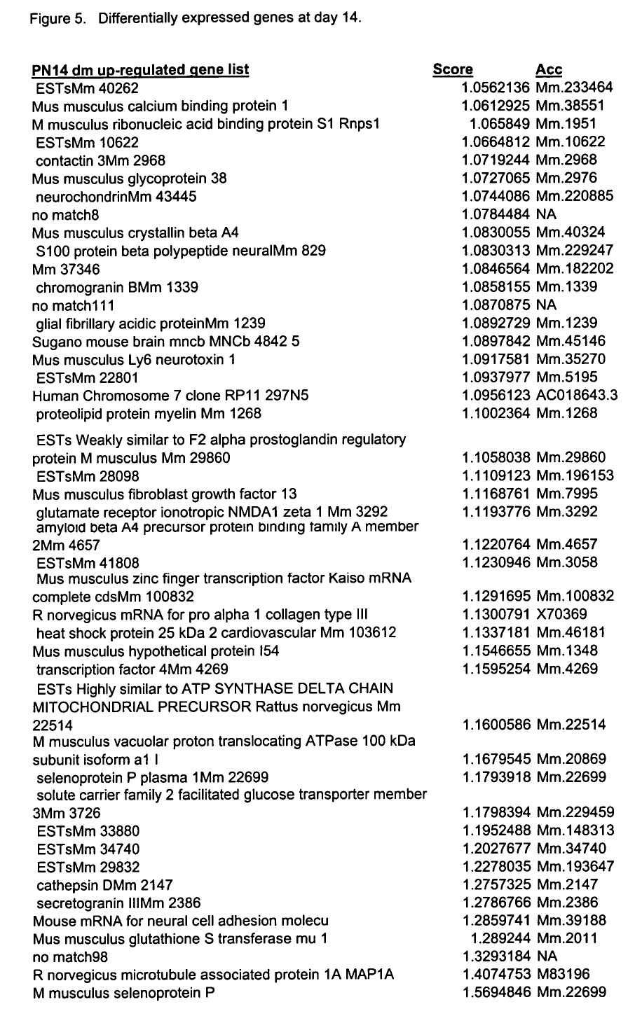

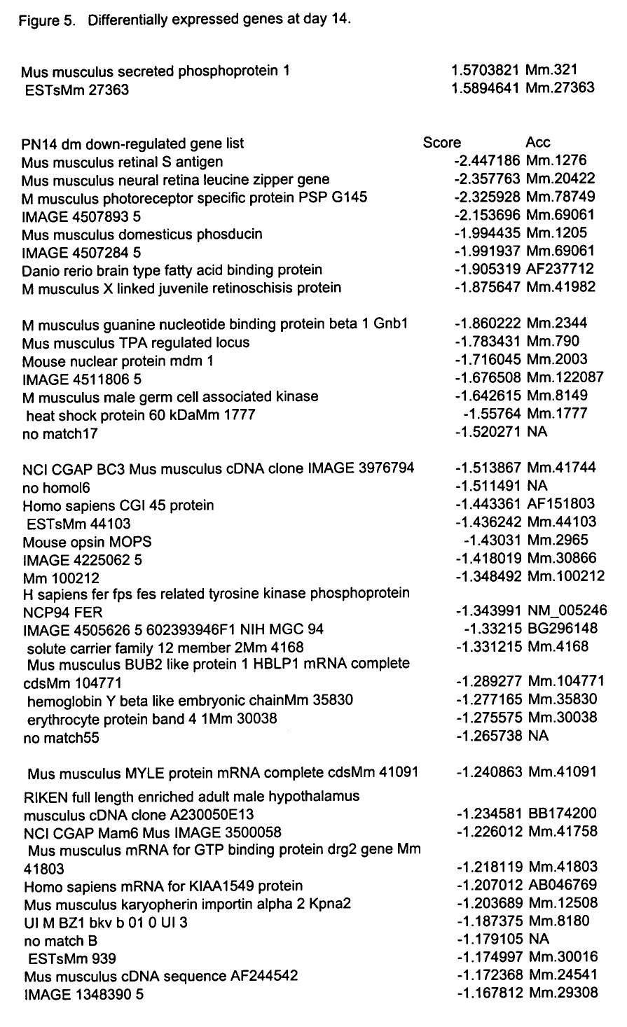

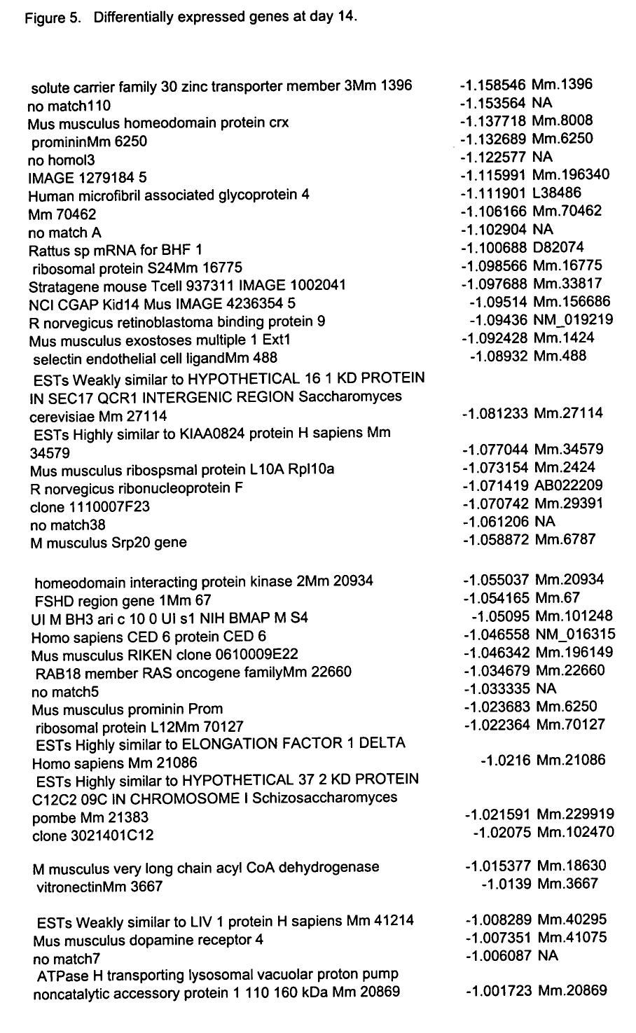

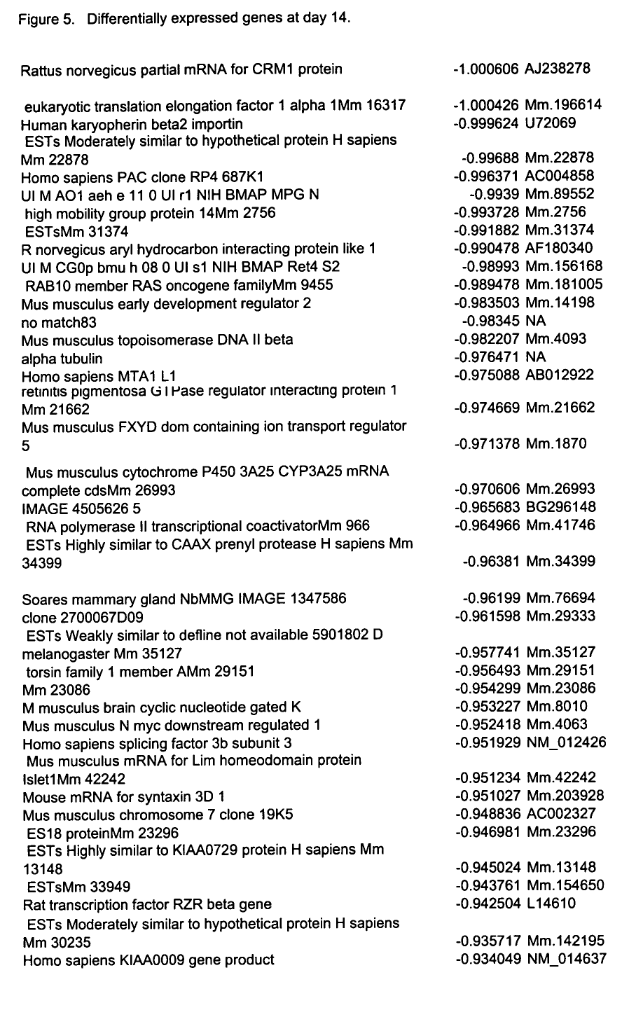

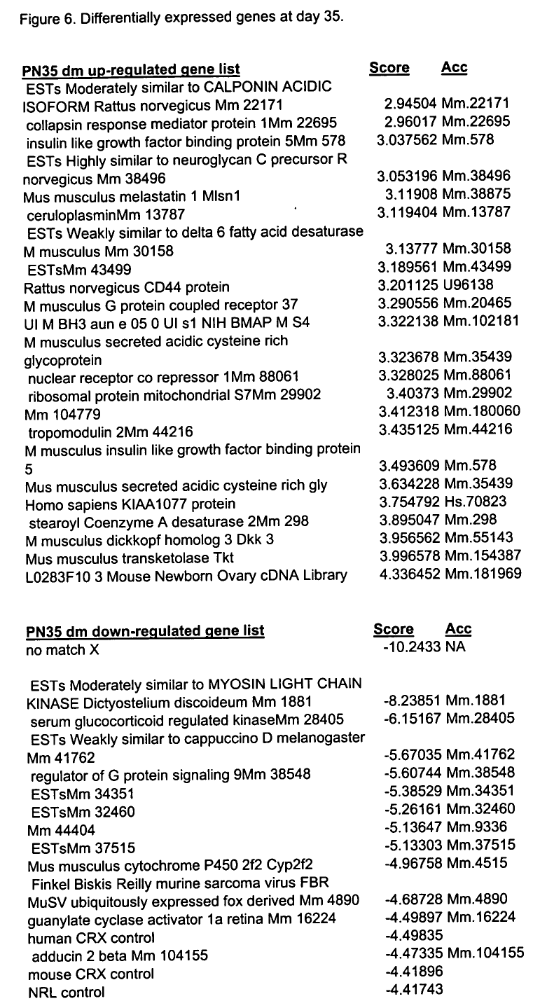

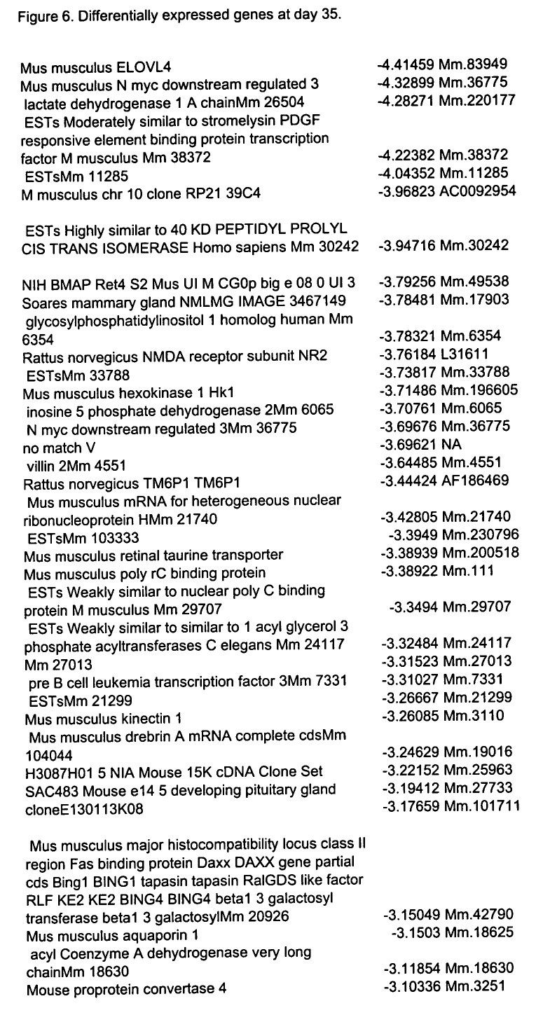

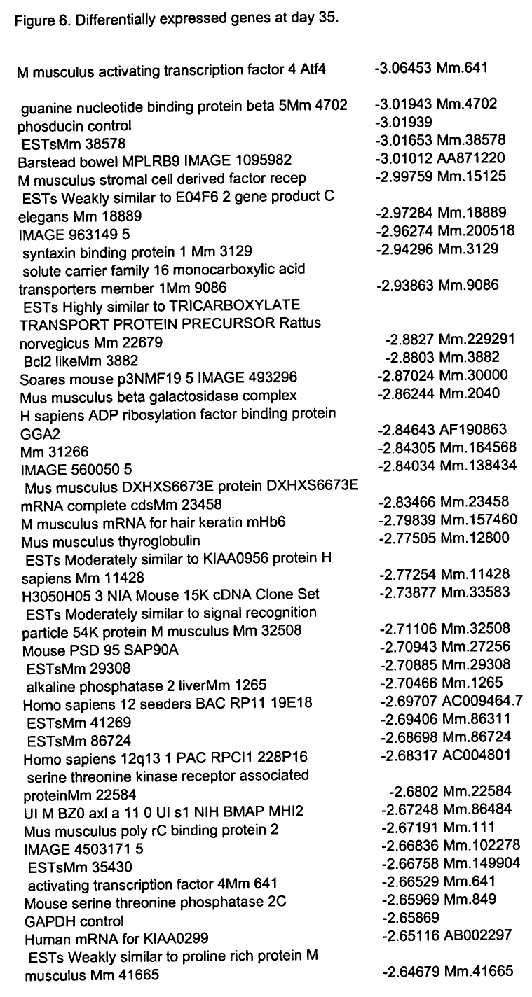

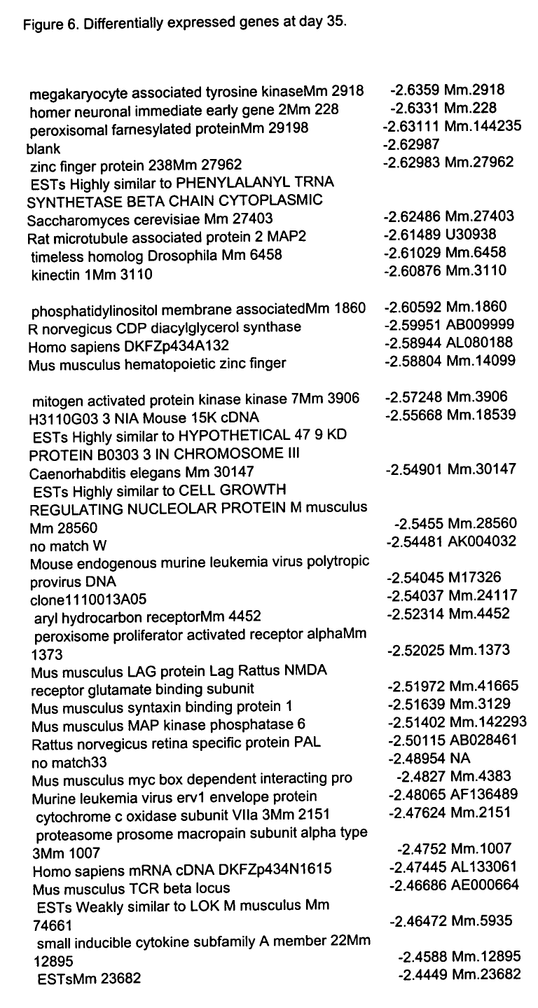

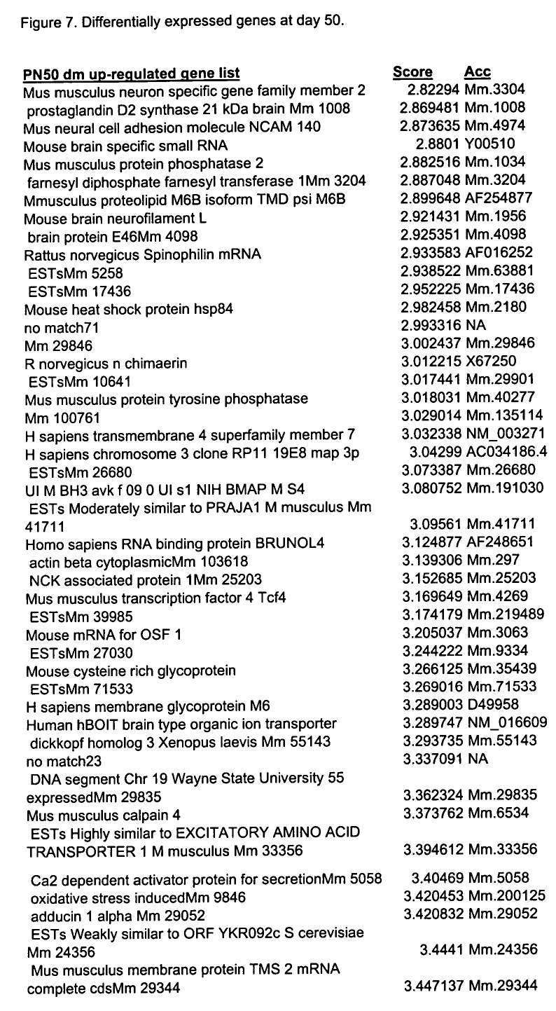

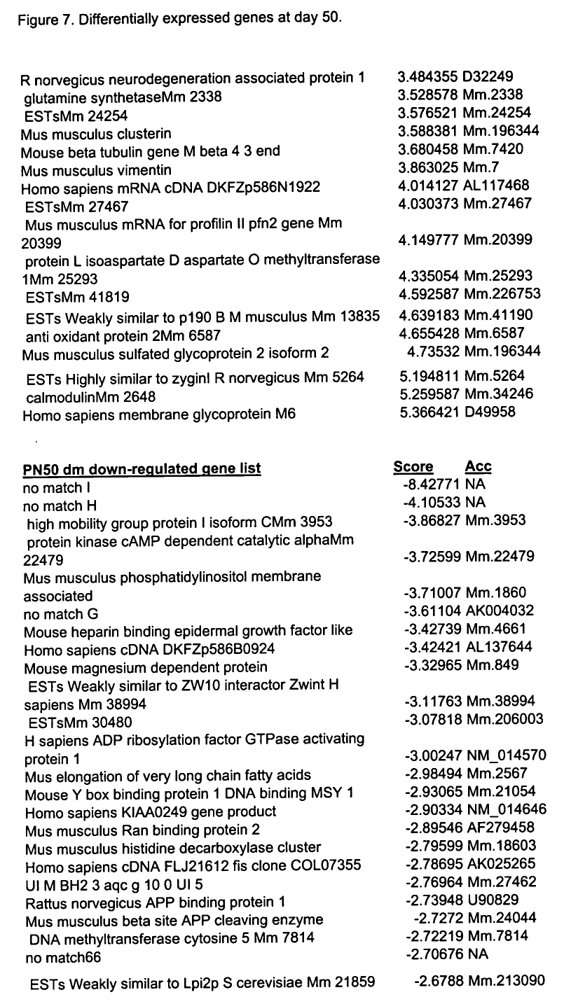

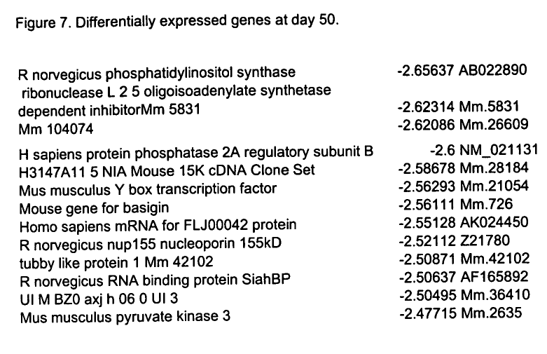

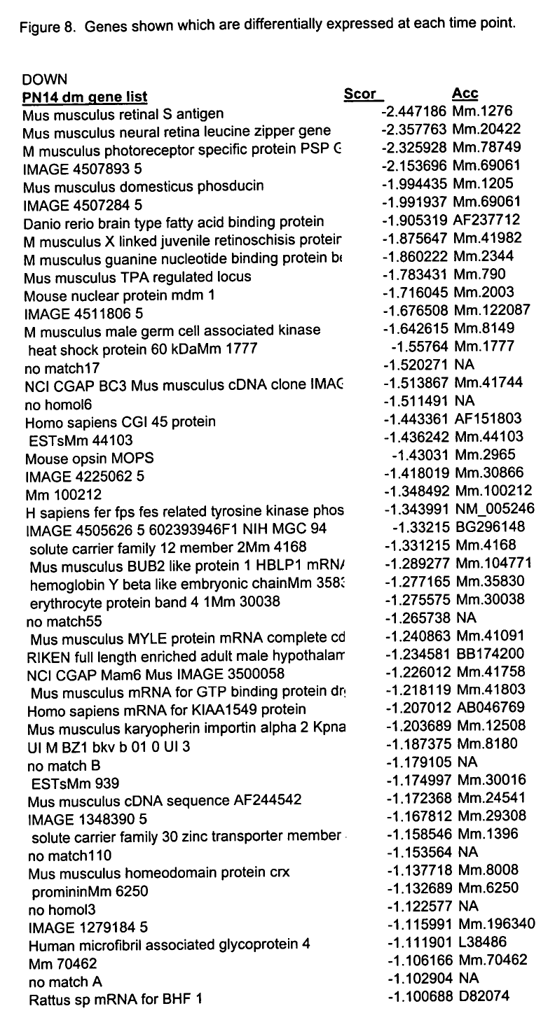

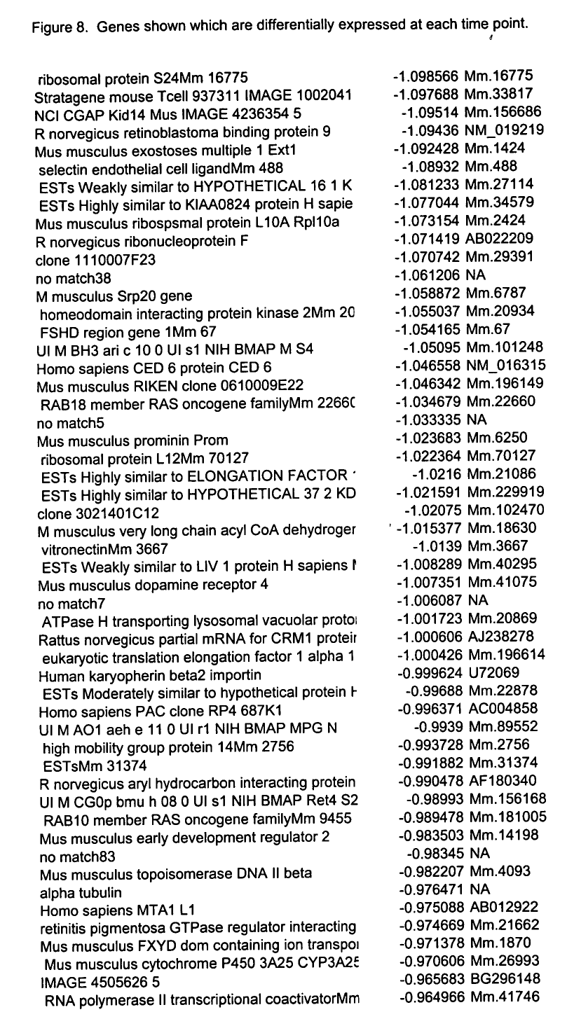

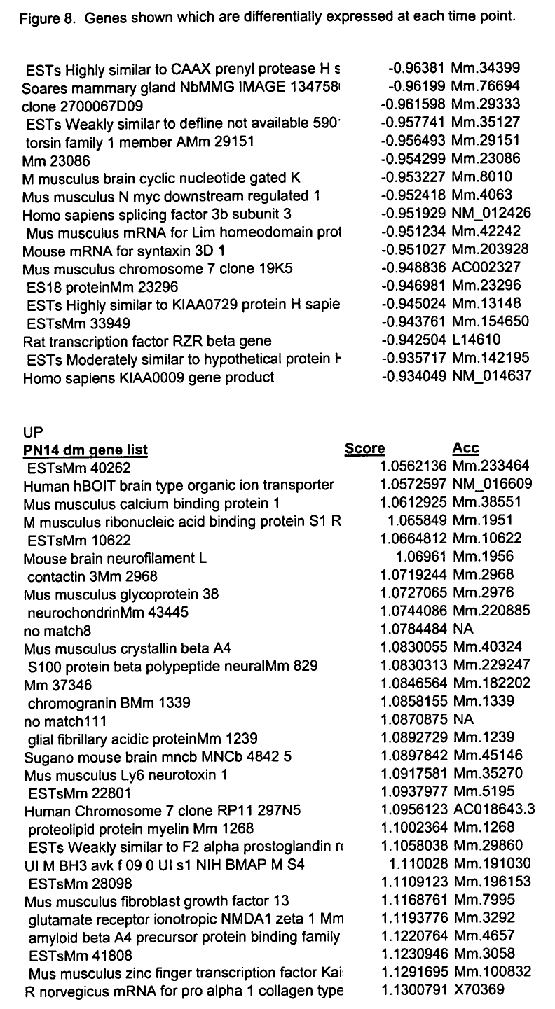

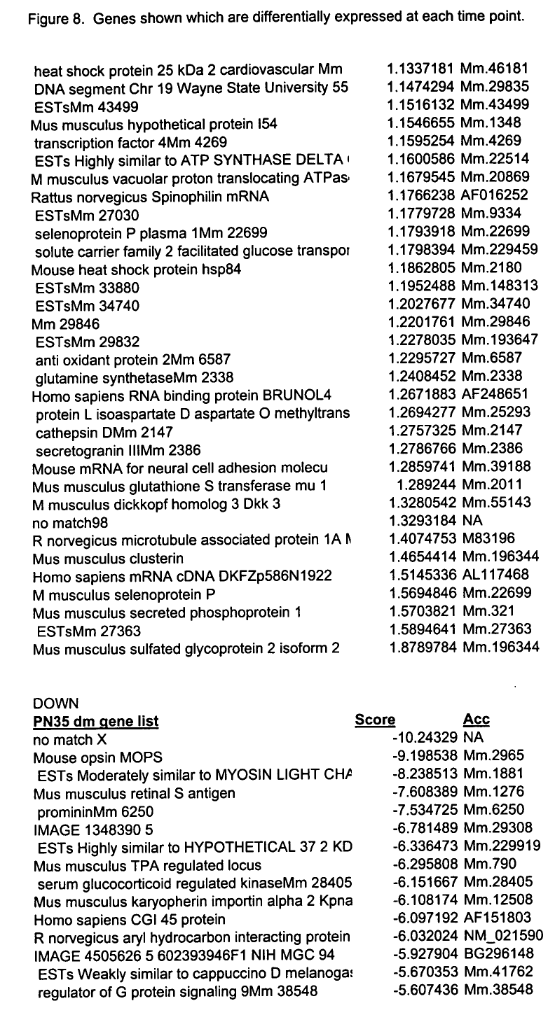

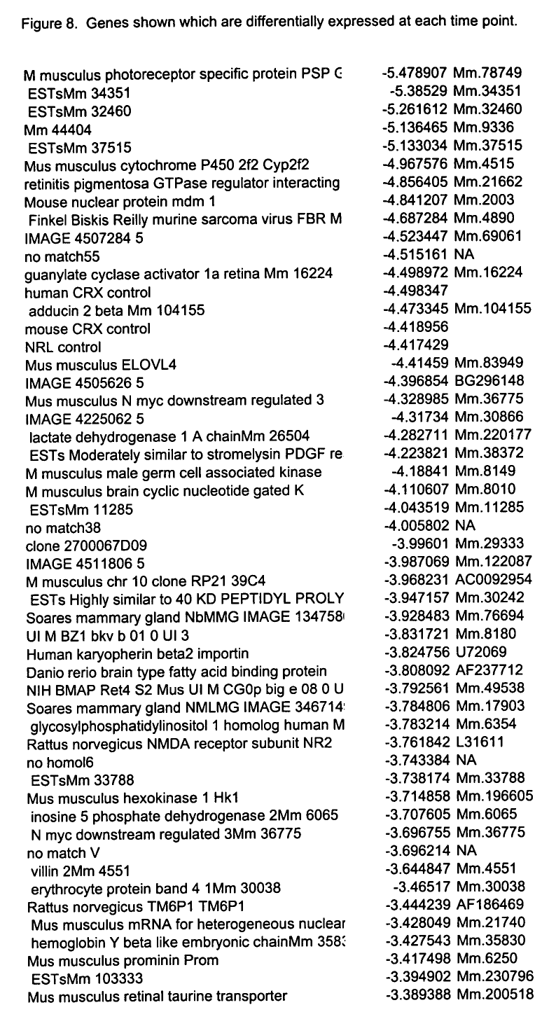

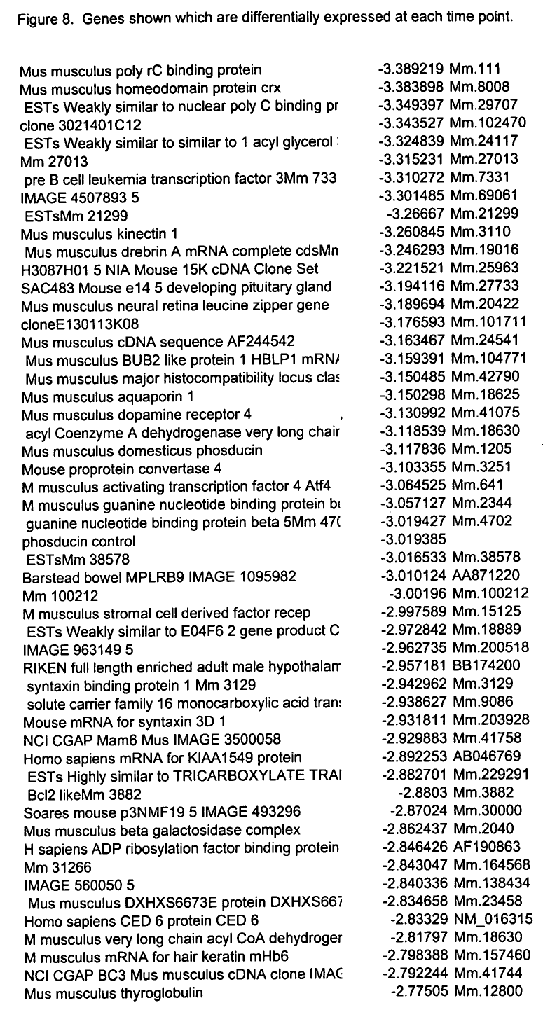

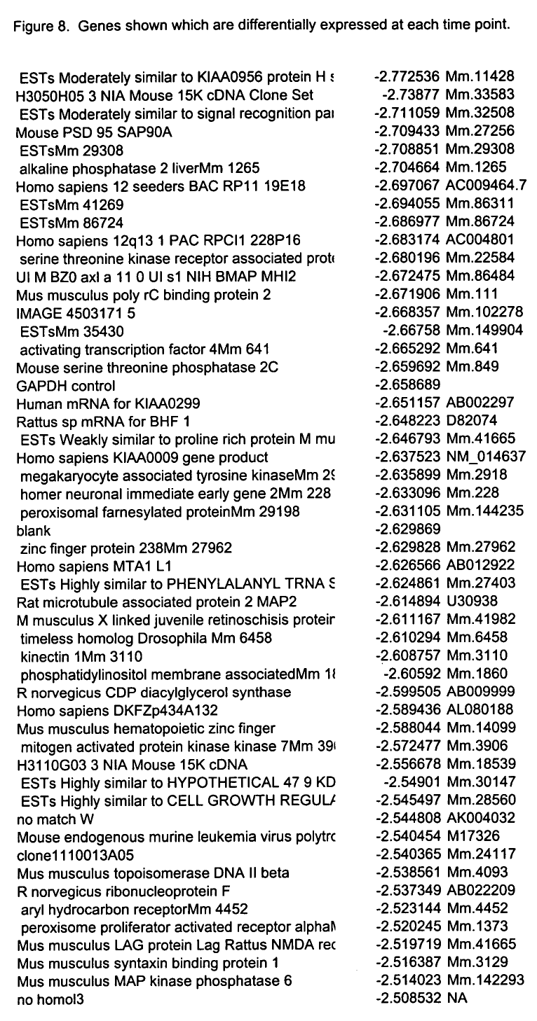

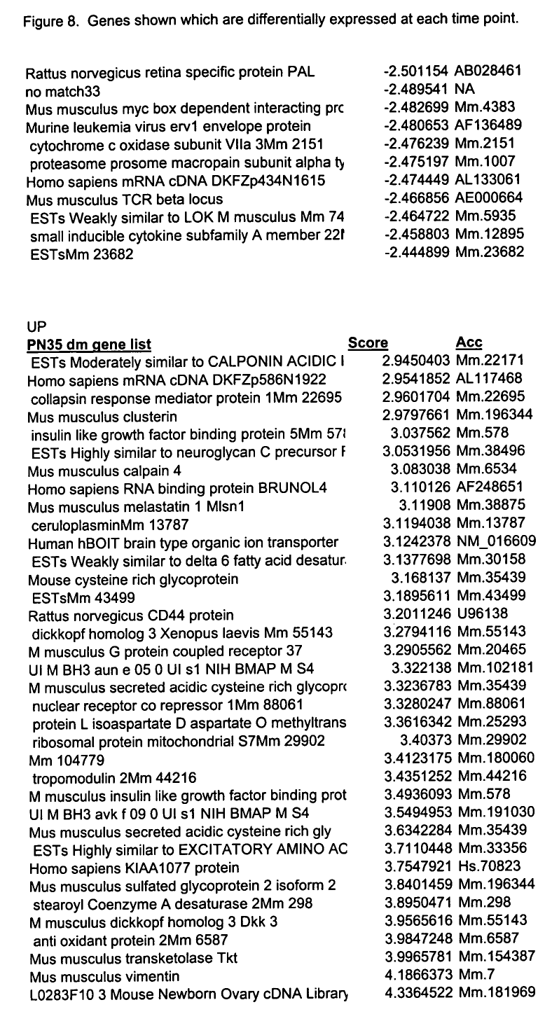

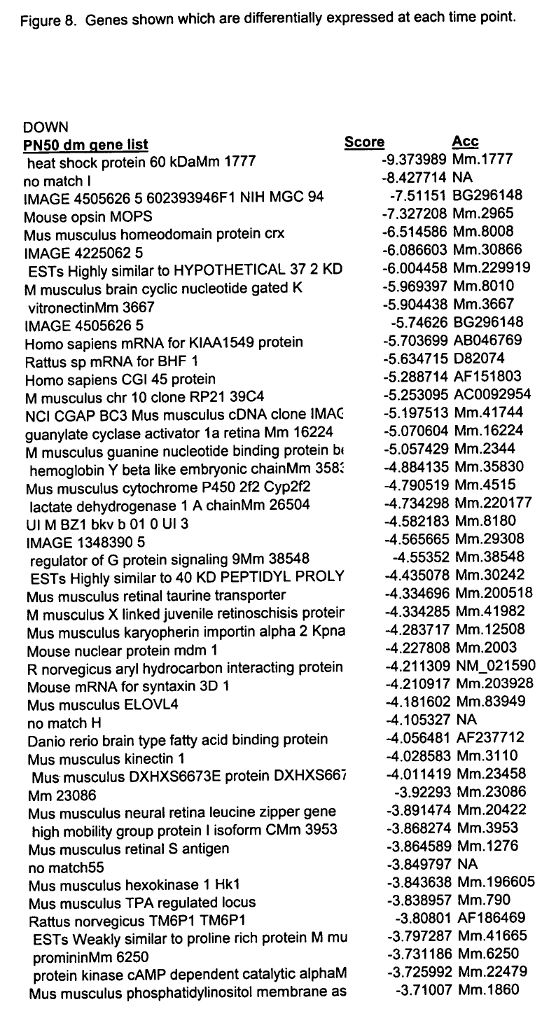

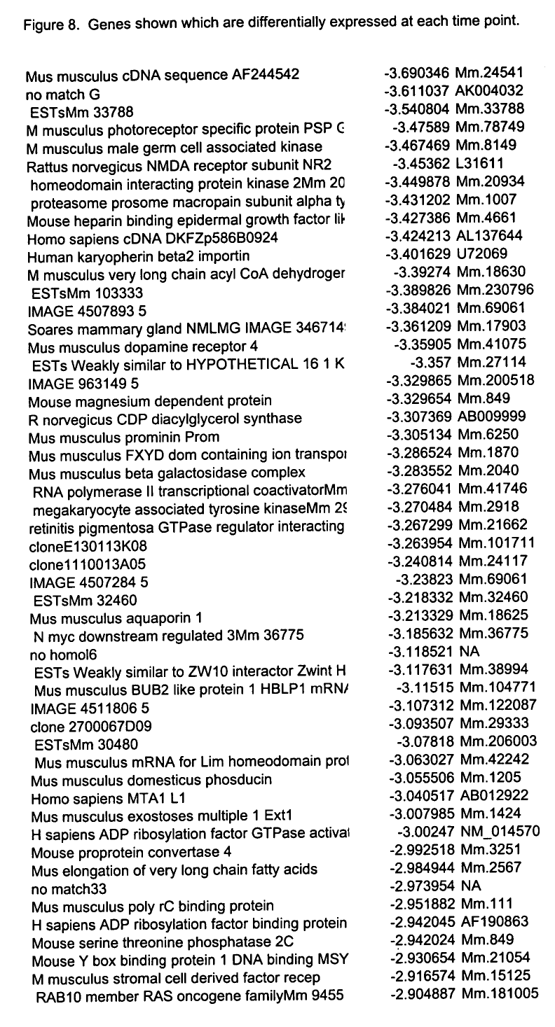

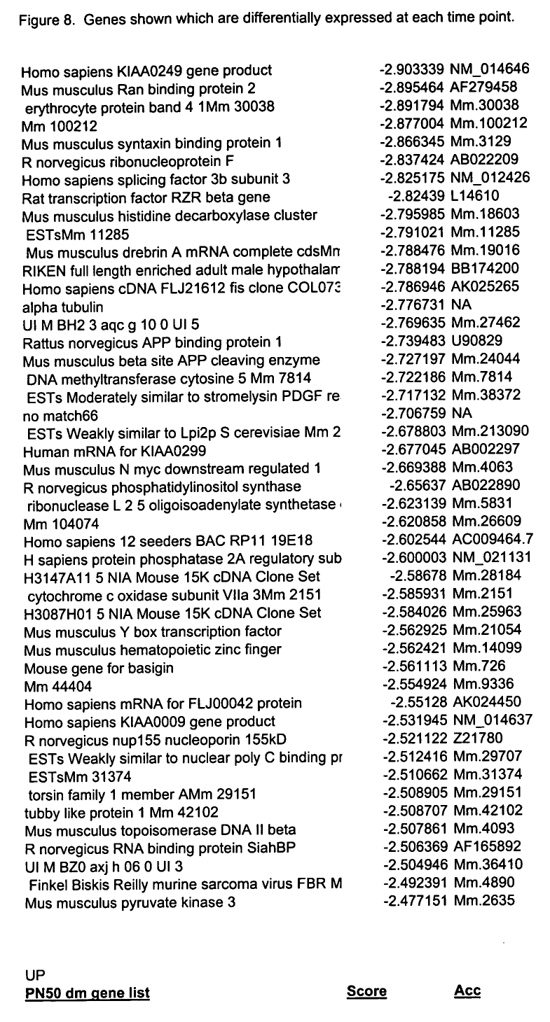

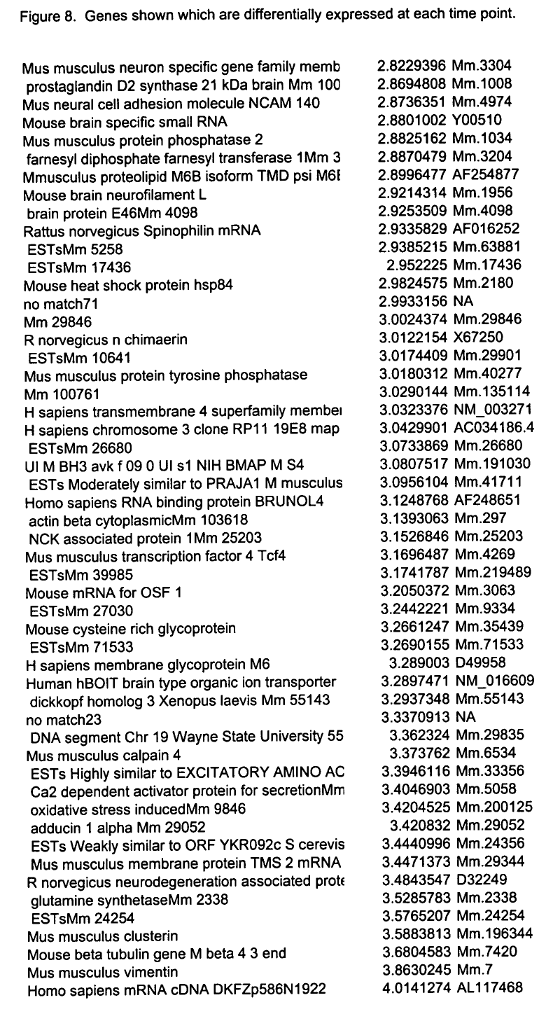

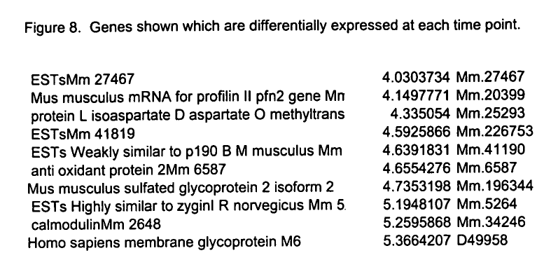

The functional breakdown of the clustered genes revealed several interesting trends (FIG. 5). Genes involved in signaling were prominent in the cluster that represented genes increased in both P35 and P50 (“up-up” and “up-no change”), but genes with roles in protein modification were not. The large “down-up” cluster mentioned above included several genes involved in oxidative stress pathways, such as superoxide dismutase and P450 oxidoreductase. The “up-down” cluster included many crystallin genes.

### EXAMPLE 7

Confirmation of Differential Expression Genes

Quantitative real-time PCR (QPCR) was used as an independent method to verify a subset of our results and to confirm the reliability of the microarray data. We tested seven genes in mutant and wildtype retinas at all three time-points. The genes were chosen to assess a range of expression ratios, including genes that had high differential expression and those with that were unchanged on the arrays. The results of the QPCR analysis are shown in Table 3. In every case, the direction of expression differences for mutant versus wildtype was replicated by QPCR, although the absolute ratios differed between the QPCR and the array; in most cases the QPCR ratio was higher than the microarray ratio, as reported in other studies (refs). Therefore, the replication of the temporal pattern of gene expression changes by QPCR suggests that the microarray results accurately reflect biological changes.

### EXAMPLE 8

Identification and Chromosomal Localization of Novel Retina Genes

A notable feature of our custom retina array is the large proportion (over 30%) of clones that represent novel genes, including ESTs and hypothetical and predicted proteins. These clones will enable identification of genes preferentially or specifically expressed in the mouse retina. Since many of the ESTs and novel genes were clustered in the “down-down” class, which also contains many known photoreceptor-specific genes, this implies that the unknown genes may also be expressed in photoreceptors. ESTs with reduced expression in rd1 mice at P50 may represent genes expressed in rod photoreceptors, whereas ESTs with increased expression P50 may represent genes expressed in non-photoreceptor cells (which have a larger representation in degenerated retinas in the same amount of RNA used).

The clones on the array (known as well as novel) may facilitate the isolation of genes that cause hereditary retinal diseases. Therefore, to generate a list of reasonable candidate genes for retina disease, we determined the chromosomal position of the human counterpart of the genes that were differentially expressed in our analyses. The strategy of combining expression data with the chromosome position of the genes and with known retina disease loci provides a powerful suggestion of a gene's involvement in disease. As shown in Table 4, 42 of the mouse clones in the down-down and down-no change clusters had human homologues that were assigned to the chromosome locations within critical regions implicated in retina disease (Table 4). Interestingly, most of these genes are involved in neuronal signaling. There are also a several novel genes. Further analysis of these candidate genes will include in situ hybridization to determine tissue localization and screens in linked families to identify mutations.

### EXAMPLE 9

Identification of Non-Coding Retina-Enriched Gene

The expression of the RetEST04 (represented by IMAGE 4505626) was decreased up to 5-fold on the array in mutant retinas. Similarly, quantitative real-time PCR showed up to a 20-fold decrease in the rod-less rd1 retinas, suggesting that RetEST04 is normally highly expressed in photoreceptors. A comparison of retina and brain amplification showed 4-fold higher expression of this EST in retina, indicating that it is indeed retina-enriched. Database genome analyses were used for further characterization. RetEST04 belongs to a group of 24 ESTs within intron 16 of the mouse photoreceptor gene RGS9. Four of the ESTs are clustered into Unigene cluster Mm.182462 (“cluster 1”), two are clustered in Mm.100970 (“cluster 2”) and the remaining ESTs fall into 2 large clusters (“cluster 3 and 4”) but are not assigned Unigene Ids (make schematic USCS figure?). The entire group of ESTs forms a 4.7 kb region, with small gaps between the clusters. Further online genome analyses (UCSC, NCBI and Ensemble) suggested that these ESTs represent a non-coding RNA. First, there was no predicted open reading frame or splice sites within this region. Homologous ESTs were identified in mouse, rat and human. Furthermore, there was no indication that these ESTs are part of RGS9 splice-forms in any species. Finally, PCR amplification on retina cDNA was did not result in consistent amplification from RGS9 to the EST clusters, again suggesting that these ESTs are not in the same transcript as the RGS9 (d.n.s.). To our knowledge, this is the first example of a retina-enriched non-coding RNA.

References

The disclosure of each reference cited is expressly incorporated herein.

Bhat, S P, Nagineni, C N (1989) αB subunit of lens-specific protein α-crystallin is present in other ocular and non-ocular tissues Biochem Biophys Res Commun 58,319-325

Kantorow, M, Horwitz, J, Sergeev, Y, Hejtmancik, J F, Piatigorsky, J. (1997) Extralenticular expression, cAMP-dependent, and cAMP-independent phosphorylation of βB2-crystallin [ARVO Abstract] Invest Ophthalmol Vis Sci 38(4),S205Abstract nr 998.

Investigative Ophthalmology and Visual Science. 2000;41 :3056-3060.) Expression of βB2-Crystallin mRNA and Protein in Retina, Brain, and Testis

Kristine S. Magabol, Joseph Horwitz, Joram Piatigorsky and Marc Kantorow, Invest Ophthalmol Vis Sci November 2002;43(11):3575-82

Retinal pigment epithelium is protected against apoptosis by alphaB-crystallin. Alge C S, Priglinger S G, Neubauer A S, Kampik A, Zillig M, Bloemendal H, Welge-Lussen U. J Biol Chem Nov. 16, 2001;276(46):43435-45

Human bcl-2 gene attenuates the ability of rabbit lens epithelial cells against H2O2-induced apoptosis through down-regulation of the alpha B-crystallin gene. Mao Y W, Xiang H, Wang J, Korsmeyer S, Reddan J, Li D W. Biochem Biophys Res Commun Oct. 4, 2002;297(4):722-8

M6a acts as a nerve growth factor-gated Ca(2+) channel in neuronal differentiation. Mukobata S, Hibino T, Sugiyama A, Urano Y, Inatomi A, Kanai Y, Endo H, Tashiro F. Reproduction October 2001;122(4):553-9

Expression of prostaglandin D synthetase during development in the mouse testis. Baker P J, O'Shaughnessy P J. Glia May 2003;42(3):263-74

Hematopoietic prostaglandin D synthase is expressed in microglia in the developing postnatal mouse brain. Mohri I, Eguchi N, Suzuki K, Urade Y, Taniike M. Invest Ophthalmol Vis Sci October 1999;40(11):2724-34

Glial cell line-derived neurotrophic factor induces histologic and functional protection of rod photoreceptors in the rd/rd mouse. Frasson M, Picaud S, Leveillard T, Simonutti M, Mohand-Said S, Dreyfus H, Hicks D, Sabel J. Lewis, G P, Matsumoto, B, Fisher, S K (1995) Changes in the organization of cytoskeletal proteins during retinal degeneration induced by retinal detachment Invest Ophthalmol Vis Sci 36,2404-2416

J Neurobiol November 1996;31(3):283-96

Localization of pleiotrophin and its mRNA in subpopulations of neurons and their corresponding axonal tracts suggests important roles in neural-glial interactions during development and in maturity. Silos-Santiago I, Yeh H J, Gurrieri M A, Guillerman R P, Li Y S, Wolf J, Snider W, Deuel T F. Eur J Neurosci. May 2002;15(9):1433-43.

1. A method of inhibiting neuronal cell death, comprising:
administering to a subject in need thereof an effective amount of an isolated molecule comprising an antibody variable region which specifically binds to a neuronal marker (NM) protein selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP1 1 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6, whereby neuronal cell death is inhibited.

2. The method of claim 1 wherein the subject has retinal cell degeneration.

3. The method of claim 1 wherein the subject has Alzheimer's disease.

4. The method of claim 1 wherein the subject has diabetic retinopathy.

5. The method of claim 1 wherein the subject has Huntington's disease.

6. The method of claim 1 wherein the subject has spinal cord injury.

7. The method of claim 1 wherein the subject has Parkinson's disease.

8. The method of claim 1 wherein the subject has glaucoma.

9. The method of claim 1 wherein the subject has age-related macular degeneration.

10. A method of preventing neuronal cell death in a mammal, comprising:
administering to the mammal a nucleic acid molecule comprising a coding sequence for a neuronal marker (NM) protein selected from the group consisting of: NM Mus musculus retinal S antigen; Mus musculus neural retina leucine zipper gene; M musculus photoreceptor specific protein PSP G145; IMAGE 4507893 5; Mus musculus domesticus phosducin; IMAGE 4507284 5; Danio rerio brain type fatty acid binding protein; M musculus X linked juvenile retinoschisis protein; M musculus guanine nucleotide binding protein beta 1 Gnb 1; Mus musculus TPA regulated locus; Mouse nuclear protein mdm 1; IMAGE 4511806 5; M musculus male germ cell associated kinase; heat shock protein 60 kDaMm 1777; no match17; NCI CGAP BC3 Mus musculus cDNA clone IMAGE 3976794; no homo16; Homo sapiens CGI 45 protein; ESTsMm 44103; Mouse opsin MOPS; IMAGE 4225062 5; Mm 100212; H sapiens fer fps fes related tyrosine kinase phosphoprotein NCP94 FER; IMAGE 4505626 5 602393946F1 NIH MGC 94; solute carrier family 12 member 2Mm 4168; Mus musculus BUB2 like protein 1 HBLP1 mRNA complete cdsMm 104771; hemoglobin Y beta like embryonic chainMm 35830; erythrocyte protein band 4 1Mm 30038; no match55; Mus musculus MYLE protein mRNA complete cdsMm 41091; RIKEN full length enriched adult male hypothalamus musculus cDNA clone A230050E13; NCI CGAP Mam6 Mus IMAGE 3500058; Mus musculus mRNA for GTP binding protein drg2 gene Mm 41803; Homo sapiens mRNA for KIAA1549 protein; Mus musculus karyopherin importin alpha 2 Kpna2; UI M BZ1 bkv b 01 0 UI 3; no match B; ESTsMm 939; Mus musculus cDNA sequence AF244542; IMAGE 1348390 5; solute carrier family 30 zinc transporter member 3Mm 1396; no match110; Mus musculus homeodomain protein crx; promininMm 6250; no homo13; IMAGE 1279184 5; Human microfibril associated glycoprotein 4; Mm 70462; no match A; Rattus sp mRNA for BHF 1; ribosomal protein S24Mm 16775; Stratagene mouse Tcell 937311 IMAGE 1002041; NCI CGAP Kid 14 Mus IMAGE 4236354 5; R norvegicus retinoblastoma binding protein 9; Mus musculus exostoses multiple 1 Ext1; selectin endothelial cell ligandMm 488; ESTs Weakly similar to HYPOTHETICAL 16 1 KD PROTEIN IN SEC17 QCR1 INTERGENIC REGION Saccharomyces cerevisiae Mm 27114; ESTs Highly similar to KIAA0824 protein H sapiens Mm 34579; Mus musculus ribospsmal protein L10A Rp110a; R norvegicus ribonucleoprotein F; clone 11 10007F23; no match38; M musculus Srp20 gene; homeodomain interacting protein kinase 2Mm 20934; FSHD region gene 1Mm 67; UI M BH3 ari c 10 0 UI s1 NIH BMAP M S4; Homo sapiens CED 6 protein CED 6; Mus musculus RIKEN clone 0610009E22; RAB18 member RAS oncogene familyMm 22660; no match5; Mus musculus prominin Prom; ribosomal protein L12Mm 70127; ESTs Highly similar to ELONGATION FACTOR 1 DELTA Homo sapiens Mm 21086; ESTs Highly similar to HYPOTHETICAL 37 2 KD PROTEIN C12C2 09C IN CHROMOSOME I Schizosaccharomyces pombe Mm 21383; clone 3021401C12; M musculus very long chain acyl CoA dehydrogenase; vitronectinMm 3667; ESTs Weakly similar to LIV 1 protein H sapiens Mm 41214; Mus musculus dopamine receptor 4; no match7; ATPase H transporting lysosomal vacuolar proton pump noncatalytic accessory protein 1 110 160 kDa Mm 20869; Rattus norvegicus partial mRNA for CRM1 protein; eukaryotic translation elongation factor 1 alpha 1Mm 16317; Human karyopherin beta2 importin; ESTs Moderately similar to hypothetical protein H sapiens Mm 22878; Homo sapiens PAC clone RP4 687K1; UI M AO1 aeh e 11 0 UI r1 NIH BMAP MPG N; high mobility group protein 14Mm 2756; ESTsMm 31374; R norvegicus aryl hydrocarbon interacting protein like 1; UI M CG0p bmu h 08 0 UI s1 NIH BMAP Ret4 S2; RAB10 member RAS oncogene familyMm 9455; Mus musculus early development regulator 2; no match83; Mus musculus topoisomerase DNA II beta; alpha tubulin; Homo sapiens MTA1 L1; retinitis pigmentosa GTPase regulator interacting protein 1 Mm 21662; Mus musculus FXYD dom containing ion transport regulator 5; Mus musculus cytochrome P450 3A25 CYP3A25 mRNA complete cdsMm 26993; IMAGE 4505626 5; RNA polymerase II transcriptional coactivatorMm 966; ESTs Highly similar to CAAX prenyl protease H sapiens Mm 34399; Soares mammary gland NbMMG IMAGE 1347586; clone 2700067D09; ESTs Weakly similar to defline not available 5901802 D melanogaster Mm 35127; torsin family 1 member AMm 29151; Mm 23086; M musculus brain cyclic nucleotide gated K; Mus musculus N myc downstream regulated 1; Homo sapiens splicing factor 3b subunit 3; Mus musculus mRNA for Lim homeodomain protein Islet1Mm 42242; Mouse mRNA for syntaxin 3D 1; Mus musculus chromosome 7 clone 19K5; ES 18 proteinMm 23296; ESTs Highly similar to KIAA0729 protein H sapiens Mm 13148; ESTsMm 33949; Rat transcription factor RZR beta gene; ESTs Moderately similar to hypothetical protein H sapiens Mm 30235; Homo sapiens KIAA0009 gene product; no match X; ESTs Moderately similar to MYOSIN LIGHT CHAIN KINASE Dictyostelium discoideum Mm 1881; serum glucocorticoid regulated kinaseMm 28405; ESTs Weakly similar to cappuccino D melanogaster Mm 41762; regulator of G protein signaling 9Mm 38548; ESTsMm 34351; ESTsMm 32460; Mm 44404; ESTsMm 37515; Mus musculus cytochrome P450 2f2 Cyp2f2; Finkel Biskis Reilly murine sarcoma virus FBR MuSV ubiquitously expressed fox derived Mm 4890; guanylate cyclase activator 1 a retina Mm 16224; human CRX control; adducin 2 beta Mm 104155; mouse CRX control; NRL control; Mus musculus ELOVL4; Mus musculus N myc downstream regulated 3; lactate dehydrogenase 1 A chainMm 26504; ESTs Moderately similar to stromelysin PDGF responsive element binding protein transcription factor M musculus Mm 38372; ESTsMm 11285; M musculus chr 10 clone RP21 39C4; ESTs Highly similar to 40 KD PEPTIDYL PROLYL CIS TRANS ISOMERASE Homo sapiens Mm 30242; NIH BMAP Ret4 S2 Mus UI M CG0p big e 08 0 UI 3; Soares mammary gland NMLMG IMAGE 3467149; glycosylphosphatidylinositol 1 homolog human Mm 6354; Rattus norvegicus NMDA receptor subunit NR2; ESTsMm 33788; Mus musculus hexokinase 1 Hk1; inosine 5 phosphate dehydrogenase 2Mm 6065; N myc downstream regulated 3Mm 36775; no match V; villin 2Mm 4551; Rattus norvegicus TM6P1 TM6P1; Mus musculus mRNA for heterogeneous nuclear ribonucleoprotein HMm 21740; ESTsMm 103333; Mus musculus retinal taurine transporter; Mus musculus poly rC binding protein; ESTs Weakly similar to nuclear poly C binding protein M musculus Mm 29707; ESTs Weakly similar to similar to 1 acyl glycerol 3 phosphate acyltransferases C elegans Mm 24117; Mm 27013; pre B cell leukemia transcription factor 3Mm 7331; ESTsMm 21299; Mus musculus kinectin 1; Mus musculus drebrin A mRNA complete cdsMm 104044; H3087H01 5 NIA Mouse 15K cDNA Clone Set; SAC483 Mouse e14 5 developing pituitary gland; cloneE130113K08; Mus musculus major histocompatibility locus class II region Fas binding protein Daxx DAXX gene partial cds Bing1 BING1 tapasin tapasin RalGDS like factor RLF KE2 KE2 BING4 BING4 beta1 3 galactosyl transferase beta1 3 galactosylMm 20926; Mus musculus aquaporin 1; acyl Coenzyme A dehydrogenase very long chainMm 18630; Mouse proprotein convertase 4; M musculus activating transcription factor 4 Atf4; guanine nucleotide binding protein beta 5Mm 4702; phosducin control; ESTsMm 38578; Barstead bowel MPLRB9 IMAGE 1095982; M musculus stromal cell derived factor recep; ESTs Weakly similar to E04F6 2 gene product C elegans Mm 18889; IMAGE 963149 5; syntaxin binding protein 1 Mm 3129; solute carrier family 16 monocarboxylic acid transporters member 1Mm 9086; ESTs Highly similar to TRICARBOXYLATE TRANSPORT PROTEIN PRECURSOR Rattus norvegicus Mm 22679; Bcl2 likeMm 3882; Soares mouse p3NMF19 5 IMAGE 493296; Mus musculus beta galactosidase complex; H sapiens ADP ribosylation factor binding protein GGA2; Mm 31266; IMAGE 560050 5; Mus musculus DXHXS6673E protein DXHXS6673E mRNA complete cdsMm 23458; M musculus mRNA for hair keratin mHb6; Mus musculus thyroglobulin; ESTs Moderately similar to KIAA0956 protein H sapiens Mm 11428; H3050H05 3 NIA Mouse 15K cDNA Clone Set; ESTs Moderately similar to signal recognition particle 54K protein M musculus Mm 32508; Mouse PSD 95 SAP90A; ESTsMm 29308; alkaline phosphatase 2 liverMm 1265; Homo sapiens 12 seeders BAC RP11 19E18; ESTsMm 41269; ESTsMm 86724; Homo sapiens 12q13 1 PAC RPCI1 228P16; serine threonine kinase receptor associated proteinMm 22584; UI M BZ0 axl a 11 0 UI s1 NIH BMAP MHI2; Mus musculus poly rC binding protein 2; IMAGE 4503171 5; ESTsMm 35430; activating transcription factor 4Mm 641; Mouse serine threonine phosphatase 2C; GAPDH control; Human mRNA for KIAA0299; ESTs Weakly similar to proline rich protein M musculus Mm 41665; megakaryocyte associated tyrosine kinaseMm 2918; homer neuronal immediate early gene 2Mm 228; peroxisomal farnesylated proteinMm 29198; blank; zinc finger protein 238Mm 27962; ESTs Highly similar to PHENYLALANYL TRNA SYNTHETASE BETA CHAIN CYTOPLASMIC Saccharomyces cerevisiae Mm 27403; Rat microtubule associated protein 2 MAP2; timeless homolog Drosophila Mm 6458; kinectin 1 Mm 3110; phosphatidylinositol membrane associatedMm 1860; R norvegicus CDP diacylglycerol synthase; Homo sapiens DKFZp434A132; Mus musculus hematopoietic zinc finger; mitogen activated protein kinase kinase 7Mm 3906; H3110G03 3 NIA Mouse 15K cDNA; ESTs Highly similar to HYPOTHETICAL 47 9 KD PROTEIN B0303 3 IN CHROMOSOME III Caenorhabditis elegans Mm 30147; ESTs Highly similar to CELL GROWTH REGULATING NUCLEOLAR PROTEIN M musculus Mm 28560; no match W; Mouse endogenous murine leukemia virus polytropic provirus DNA; clone1110013A05; aryl hydrocarbon receptorMm 4452; peroxisome proliferator activated receptor alphaMm 1373; Mus musculus LAG protein Lag Rattus NMDA receptor glutamate binding subunit; Mus musculus syntaxin binding protein 1; Mus musculus MAP kinase phosphatase 6; Rattus norvegicus retina specific protein PAL; no match33; Mus musculus myc box dependent interacting pro; Murine leukemia virus erv1 envelope protein; cytochrome c oxidase subunit VIIa 3Mm 2151; proteasome prosome macropain subunit alpha type 3Mm 1007; Homo sapiens mRNA cDNA DKFZp434N1615; Mus musculus TCR beta locus; ESTs Weakly similar to LOK M musculus Mm 74661; small inducible cytokine subfamily A member 22Mm 12895; ESTsMm 23682; no match I; no match H; high mobility group protein I isoform CMm 3953; protein kinase cAMP dependent catalytic alphaMm 22479; Mus musculus phosphatidylinositol membrane associated; no match G; Mouse heparin binding epidermal growth factor like; Homo sapiens cDNA DKFZp586B0924; Mouse magnesium dependent protein; ESTs Weakly similar to ZW10 interactor Zwint H sapiens Mm 38994; ESTsMm 30480; H sapiens ADP ribosylation factor GTPase activating protein 1; Mus elongation of very long chain fatty acids; Mouse Y box binding protein 1 DNA binding MSY 1; Homo sapiens KIAA0249 gene product; Mus musculus Ran binding protein 2; Mus musculus histidine decarboxylase cluster; Homo sapiens cDNA FLJ21612 fis clone COL07355; UI M BH2 3 aqc g 10 0 UI 5; Rattus norvegicus APP binding protein 1; Mus musculus beta site APP cleaving enzyme; DNA methyltransferase cytosine 5 Mm 7814; no match66; ESTs Weakly similar to Lpi2p S cerevisiae Mm 21859; R norvegicus phosphatidylinositol synthase; ribonuclease L 2 5 oligoisoadenylate synthetase dependent inhibitorMm 5831; Mm 104074; H sapiens protein phosphatase 2A regulatory subunit B; H3147A11 5 NIA Mouse 15K cDNA Clone Set; Mus musculus Y box transcription factor; Mouse gene for basigin; Homo sapiens mRNA for FLJ00042 protein; R norvegicus nup155 nucleoporin 155kD; tubby like protein 1 Mm 42102; R norvegicus RNA binding protein SiahBP; UI M BZ0 axj h 06 0 UI3; and Mus musculus pyruvate kinase 3, whereby neuronal cell death in the mammal is inhibited or prevented.

11. A method of preventing neuronal cell death in a mammal, comprising:
administering to the mammal a purified human neuronal marker (NM) protein selected from the group consisting of: NM Mus musculus retinal S antigen; Mus musculus neural retina leucine zipper gene; M musculus photoreceptor specific protein PSP G145; IMAGE 4507893 5; Mus musculus domesticus phosducin; IMAGE 4507284 5; Danio rerio brain type fatty acid binding protein; M musculus X linked juvenile retinoschisis protein; M musculus guanine nucleotide binding protein beta 1 Gnb1; Mus musculus TPA regulated locus; Mouse nuclear protein mdm 1; IMAGE 4511806 5; M musculus male germ cell associated kinase; heat shock protein 60 kDaMm 1777; no match17; NCI CGAP BC3 Mus musculus cDNA clone IMAGE 3976794; no homo16; Homo sapiens CGI 45 protein; ESTsMm 44103; Mouse opsin MOPS; IMAGE 4225062 5; Mm 100212; H sapiens fer fps fes related tyrosine kinase phosphoprotein NCP94 FER; IMAGE 4505626 5 602393946F1 NIH MGC 94; solute carrier family 12 member 2Mm 4168; Mus musculus BUB2 like protein 1 HBLP1 mRNA complete cdsMm 104771; hemoglobin Y beta like embryonic chainMm 35830; erythrocyte protein band 4 1Mm 30038; no match55; Mus musculus MYLE protein mRNA complete cdsMm 41091; RIKEN full length enriched adult male hypothalamus musculus cDNA clone A230050E13; NCI CGAP Mam6 Mus IMAGE 3500058; Mus musculus mRNA for GTP binding protein drg2 gene Mm 41803; Homo sapiens mRNA for KIAA1549 protein; Mus musculus karyopherin importin alpha 2 Kpna2; UI M BZ1 bkv b 01 0 UI 3; no match B; ESTsMm 939; Mus musculus cDNA sequence AF244542; IMAGE 1348390 5; solute carrier family 30 zinc transporter member 3Mm 1396; no match110; Mus musculus homeodomain protein crx; promininMm 6250; no homo13; IMAGE 1279184 5; Human microfibril associated glycoprotein 4; Mm 70462; no match A; Rattus sp mRNA for BHF 1; ribosomal protein S24Mm 16775; Stratagene mouse Tcell 937311 IMAGE 1002041; NCI CGAP Kid14 Mus IMAGE 4236354 5; R norvegicus retinoblastoma binding protein 9; Mus musculus exostoses multiple 1 Ext1; selectin endothelial cell ligandMm 488; ESTs Weakly similar to HYPOTHETICAL 16 1 KD PROTEIN IN SEC17 QCR1 INTERGENIC REGION Saccharomyces cerevisiae Mm 27114; ESTs Highly similar to KIAA0824 protein H sapiens Mm 34579; Mus musculus ribospsmal protein L10A Rp110a; R norvegicus ribonucleoprotein F; clone 1110007F23; no match38; M musculus Srp20 gene; homeodomain interacting protein kinase 2Mm 20934; FSHD region gene 1Mm 67; UI M BH3 ari c 10 0 UI s1 NIH BMAP M S4; Homo sapiens CED 6 protein CED 6; Mus musculus RIKEN clone 0610009E22; RAB18 member RAS oncogene familyMm 22660; no match5; Mus musculus prominin Prom; ribosomal protein L12Mm 70127; ESTs Highly similar to ELONGATION FACTOR 1 DELTA Homo sapiens Mm 21086; ESTs Highly similar to HYPOTHETICAL 37 2 KD PROTEIN C12C2 09C IN CHROMOSOME I Schizosaccharomyces pombe Mm 21383; clone 3021401C12; M musculus very long chain acyl CoA dehydrogenase; vitronectinim 3667; ESTs Weakly similar to LIV 1 protein H sapiens Mm 41214; Mus musculus dopamine receptor 4; no match7; ATPase H transporting lysosomal vacuolar proton pump noncatalytic accessory protein 1 110 160 kDa Mm 20869; Rattus norvegicus partial mRNA for CRM1 protein; eukaryotic translation elongation factor 1 alpha 1Mm 16317; Human karyopherin beta2 importin; ESTs Moderately similar to hypothetical protein H sapiens Mm 22878; Homo sapiens PAC clone RP4 687K1; UI M AO1 aeh e 11 0 UI r1 NIH BMAP MPG N; high mobility group protein 14Mm 2756; ESTsMm 31374; R norvegicus aryl hydrocarbon interacting protein like 1; UI M CG0p bmu h 08 0 UI s1 NIH BMAP Ret4 S2; RAB10 member RAS oncogene familyMm 9455; Mus musculus early development regulator 2; no match83; Mus musculus topoisomerase DNA II beta; alpha tubulin; Homo sapiens MTA1 L1; retinitis pigmentosa GTPase regulator interacting protein 1 Mm 21662; Mus musculus FXYD dom containing ion transport regulator 5; Mus musculus cytochrome P450 3A25 CYP3A25 mRNA complete cdsMm 26993; IMAGE 4505626 5; RNA polymerase II transcriptional coactivatorMm 966; ESTs Highly similar to CAAX prenyl protease H sapiens Mm 34399; Soares mammary gland NbMMG IMAGE 1347586; clone 2700067D09; ESTs Weakly similar to defline not available 5901802 D melanogaster Mm 35127; torsin family 1 member AMm 29151; Mm 23086; M musculus brain cyclic nucleotide gated K; Mus musculus N myc downstream regulated 1; Homo sapiens splicing factor 3b subunit 3; Mus musculus mRNA for Lim homeodomain protein Islet1Mm 42242; Mouse mRNA for syntaxin 3D 1; Mus musculus chromosome 7 clone 19K5; ES18 proteinMm 23296; ESTs Highly similar to KIAA0729 protein H sapiens Mm 13148; ESTsMm 33949; Rat transcription factor RZR beta gene; ESTs Moderately similar to hypothetical protein H sapiens Mm 30235; Homo sapiens KIAA0009 gene product; no match X; ESTs Moderately similar to MYOSIN LIGHT CHAIN KINASE Dictyostelium discoideum Mm 1881; serum glucocorticoid regulated kinaseMm 28405; ESTs Weakly similar to cappuccino D melanogaster Mm 41762; regulator of G protein signaling 9Mm 38548; ESTsMm 34351; ESTsMm 32460; Mm 44404; ESTsMm 37515; Mus musculus cytochrome P450 2f2 Cyp2f2; Finkel Biskis Reilly murine sarcoma virus FBR MuSV ubiquitously expressed fox derived Mm 4890; guanylate cyclase activator 1a retina Mm 16224; human CRX control; adducin 2 beta Mm 104155; mouse CRX control; NRL control; Mus musculus ELOVL4; Mus musculus N myc downstream regulated 3; lactate dehydrogenase 1 A chainMm 26504; ESTs Moderately similar to stromelysin PDGF responsive element binding protein transcription factor M musculus Mm 38372; ESTsMm 11285; M musculus chr 10 clone RP21 39C4; ESTs Highly similar to 40 KD PEPTIDYL PROLYL CIS TRANS ISOMERASE Homo sapiens Mm 30242; NIH BMAP Ret4 S2 Mus UI M CG0p big e 08 0 UI 3; Soares mammary gland NMLMG IMAGE 3467149; glycosylphosphatidylinositol 1 homolog human Mm 6354; Rattus norvegicus NMDA receptor subunit NR2; ESTsMm 33788; Mus musculus hexokinase 1 Hk1; inosine 5 phosphate dehydrogenase 2Mm 6065; N myc downstream regulated 3Mm 36775; no match V; villin 2Mm 4551; Rattus norvegicus TM6P1 TM6P1; Mus musculus mRNA for heterogeneous nuclear ribonucleoprotein HMm 21740; ESTsMm 103333; Mus musculus retinal taurine transporter; Mus musculus poly rC binding protein; ESTs Weakly similar to nuclear poly C binding protein M musculus Mm 29707; ESTs Weakly similar to similar to 1 acyl glycerol 3 phosphate acyltransferases C elegans Mm 24117; Mm 27013; pre B cell leukemia transcription factor 3Mm 7331; ESTsMm 21299; Mus musculus kinectin 1; Mus musculus drebrin A mRNA complete cdsMm 104044; H3087H01 5 NIA Mouse 15K cDNA Clone Set; SAC483 Mouse e14 5 developing pituitary gland; cloneE13013K08; Mus musculus major histocompatibility locus class II region Fas binding protein Daxx DAXX gene partial cds Bing1 BING1 tapasin tapasin RalGDS like factor RLF KE2 KE2 BING4 BING4 beta1 3 galactosyl transferase beta1 3 galactosylMm 20926; Mus musculus aquaporin 1; acyl Coenzyme A dehydrogenase very long chainMm 18630; Mouse proprotein convertase 4; M musculus activating transcription factor 4 Atf4; guanine nucleotide binding protein beta 5Mm 4702; phosducin control; ESTsMm 38578; Barstead bowel MPLRB9 IMAGE 1095982; M musculus stromal cell derived factor recep; ESTs Weakly similar to E04F6 2 gene product C elegans Mm 18889; IMAGE 963149 5; syntaxin binding protein 1 Mm 3129; solute carrier family 16 monocarboxylic acid transporters member 1Mm 9086; ESTs Highly similar to TRICARBOXYLATE TRANSPORT PROTEIN PRECURSOR Rattus norvegicus Mm 22679; Bcl2 likeMm 3882; Soares mouse p3NMF19 5 IMAGE 493296; Mus musculus beta galactosidase complex; H sapiens ADP ribosylation factor binding protein GGA2; Mm 31266; IMAGE 560050 5; Mus musculus DXHXS6673E protein DXHXS6673E mRNA complete cdsMm 23458; M musculus mRNA for hair keratin mHb6; Mus musculus thyroglobulin; ESTs Moderately similar to KIAA0956 protein H sapiens Mm 11428; H305OH05 3 NIA Mouse 15K cDNA Clone Set; ESTs Moderately similar to signal recognition particle 54K protein M musculus Mm 32508; Mouse PSD 95 SAP90A; ESTsMm 29308; alkaline phosphatase 2 liverMm 1265; Homo sapiens 12 seeders BAC RP11 19E18; ESTsMm 41269; ESTsMm 86724; Homo sapiens 12q13 1 PAC RPC11 228P16; serine threonine kinase receptor associated proteinMm 22584; UI M BZ0 axl a 11 0 UI s1 NIH BMAP MHI2; Mus musculus poly rC binding protein 2; IMAGE 4503171 5; ESTsMm 35430; activating transcription factor 4Mm 641; Mouse serine threonine phosphatase 2C; GAPDH control; Human mRNA for KIAA0299; ESTs Weakly similar to proline rich protein M musculus Mm 41665; megakaryocyte associated tyrosine kinaseMm 2918; homer neuronal immediate early gene 2Mm 228; peroxisomal farnesylated proteinMm 29198; blank; zinc finger protein 238Mm 27962; ESTs Highly similar to PHENYLALANYL TRNA SYNTHETASE BETA CHAIN CYTOPLASMIC Saccharomyces cerevisiae Mm 27403; Rat microtubule associated protein 2 MAP2; timeless homolog Drosophila Mm 6458; kinectin 1Mm 3110; phosphatidylinositol membrane associatedMm 1860; R norvegicus CDP diacylglycerol synthase; Homo sapiens DKFZp434A132; Mus musculus hematopoietic zinc finger; mitogen activated protein kinase kinase 7Mm 3906; H3110G03 3 NIA Mouse 15K cDNA; ESTs Highly similar to HYPOTHETICAL 47 9 KD PROTEIN B0303 3 IN CHROMOSOME III Caenorhabditis elegans Mm 30147; ESTs Highly similar to CELL GROWTH REGULATING NUCLEOLAR PROTEIN M musculus Mm 28560; no match W; Mouse endogenous murine leukemia virus polytropic provirus DNA; clone1110013A05; aryl hydrocarbon receptorMm 4452; peroxisome proliferator activated receptor alphaMm 1373; Mus musculus LAG protein Lag Rattus NMDA receptor glutamate binding subunit; Mus musculus syntaxin binding protein 1; Mus musculus MAP kinase phosphatase 6; Rattus norvegicus retina specific protein PAL; no match33; Mus musculus myc box dependent interacting pro; Murine leukemia virus erv1 envelope protein; cytochrome c oxidase subunit VIIa 3Mm 2151; proteasome prosome macropain subunit alpha type 3Mm 1007; Homo sapiens mRNA cDNA DKFZp434N1615; Mus musculus TCR beta locus; ESTs Weakly similar to LOK M musculus Mm 74661; small inducible cytokine subfamily A member 22Mm 12895; ESTsMm 23682; no match I; no match H; high mobility group protein I isoform CMm 3953; protein kinase cAMP dependent catalytic alphaMm 22479; Mus musculus phosphatidylinositol membrane associated; no match G; Mouse heparin binding epidermal growth factor like; Homo sapiens cDNA DKFZp586B0924; Mouse magnesium dependent protein; ESTs Weakly similar to ZW10 interactor Zwint H sapiens Mm 38994; ESTsMm 30480; H sapiens ADP ribosylation factor GTPase activating protein 1; Mus elongation of very long chain fatty acids; Mouse Y box binding protein 1 DNA binding MSY 1; Homo sapiens KIAA0249 gene product; Mus musculus Ran binding protein 2; Mus musculus histidine decarboxylase cluster; Homo sapiens cDNA FLJ21612 fis clone COL07355; UI M BH2 3 aqc g 10 0 UI 5; Rattus norvegicus APP binding protein 1; Mus musculus beta site APP cleaving enzyme; DNA methyltransferase cytosine 5 Mm 7814; no match66; ESTs Weakly similar to Lpi2p S cerevisiae Mm 21859; R norvegicus phosphatidylinositol synthase; ribonuclease L-2 5 oligoisoadenylate synthetase dependent inhibitorMm 5831; Mm 104074; H sapiens protein phosphatase 2A regulatory subunit B; H3147A11 5 NIA Mouse 15K cDNA Clone Set; Mus musculus Y box transcription factor; Mouse gene for basigin; Homo sapiens mRNA for FLJ00042 protein; R norvegicus nup155 nucleoporin 155kD; tubby like protein 1 Mm 42102; R norvegicus RNA binding protein SiahBP; UI M BZ0 axj h 06 0 UI 3; and Mus musculus pyruvate kinase 3, whereby neuronal cell death in the mammal is inhibited or prevented.

12. The method of claim 10 or 11 wherein the subject has retinal cell degeneration.

13. The method of claim 10 or 11 wherein the subject has Alzheimer's disease.

14. The method of claim 10 or 11 wherein the subject has diabetic retinopathy.

15. The method of claim 10 or 11 wherein the subject has Huntington's disease.

16. The method of claim 10 or 11 wherein the subject has spinal cord injury.

17. The method of claim 10 or 11 wherein the subject has Parkinson's disease.

18. The method of claim 10 or 11 wherein the subject has glaucoma.

19. The method of claim 10 or 11 wherein the subject has age-related macular degeneration.

20. A method of identifying regions of neuronal cell death in a patient, comprising:
administering to a patient a molecule comprising an antibody variable region which specifically binds to a neuronal marker (NM) protein selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6, wherein the molecule is bound to a detectable moiety; and
detecting the detectable moiety in the patient, thereby identifying regions of neuronal cell death.

21. The method of claim 20 wherein the subject has retinal cell degeneration.

22. The method of claim 20 wherein the subject has Alzheimer's disease.

23. The method of claim 20 wherein the subject has diabetic retinopathy.

24. The method of claim 20 wherein the subject has Huntington's disease.

25. The method of claim 20 wherein the subject has spinal cord injury.

26. The method of claim 20 wherein the subject has Parkinson's disease.

27. The method of claim 20 wherein the subject has glaucoma.

28. The method of claim 20 wherein the subject has age-related macular degeneration.

29. A method of screening for neuronal cell death in a patient, comprising:
contacting a body fluid collected from the patient with a molecule comprising an antibody variable region which specifically binds to a neuronal marker (NM) protein selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP 11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e&05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6, wherein detection of cross-reactive material in the body fluid with the molecule indicates neuronal cell death in the patient.

30. A method of promoting neuronal cell death in a patient, comprising:
administering to a patient in need of neuronal cell death a neuronal marker (NM) protein selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6, whereby neuronal cell death in the patient is stimulated.

31. The method of claim 30 wherein the patient has a neuronal tumor.

32. A method of promoting neuronal cell death in a patient, comprising:
administering to a patient in need of neuronal cell death a nucleic acid molecule encoding a neuronal marker (NM) protein selected from the group consisting of ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type II; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6, whereby the NM protein is expressed and neuronal cell death in the patient is stimulated.

33. The method of claim 32 wherein the patient has a neuronal tumor.

34. A method of screening for neuronal cell death in a patient, comprising:
detecting a neuronal marker (NM) protein selected from the group consisting of ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP1 1 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP 11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6, in a body fluid collected from the patient, wherein detection of the NM protein indicates neuronal cell death in the patient.

35. A method of screening for neuronal cell death in a patient, comprising:
detecting in a body fluid collected from the patient a nucleic acid encoding a neuronal marker (NM) protein selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein I54; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6, wherein detection of the NM protein indicates neuronal cell death in the patient.

36. A method to identify candidate drugs for treating neuronal cell death, comprising:
contacting cells which express one or more neuronal marker (NM) genes selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn12 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6, with a test compound;
determining expression of said one or more NM genes by hybridization of mRNA of said cells to a nucleic acid probe which is complementary to said mRNA; and
identifying a test compound as a candidate drug for treating neuronal cell death if it decreases expression of said one or more NM genes.

37. The method of claim 36 wherein the cells are retinal cells.

38. The method of claim 36 wherein the cells are recombinant host cells which are transfected with an expression construct which encodes said one or more NMs.

39. A method to identify candidate drugs for treating neuronal cell death, comprising:
contacting cells which express one or more neuronal marker (NM) proteins selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F 10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6, with a test compound;
determining amount of said one or more NM proteins in said cells; and
identifying a test compound as a candidate drug for treating tumors if it decreases the amount of one more NM proteins in said cells.

40. The method of claim 39 wherein the cells are retinal cells.

41. The method of claim 39 wherein the cells are recombinant host cells which are transfected with an expression construct which encodes said one or more NMs.

42. A method to identify candidate drugs for treating neuronal cell death, comprising:
contacting cells which express one or more neuronal marker (NM) proteins selected from the group consisting of: ESTsMm 40262; Mus musculus calcium binding protein 1; M musculus ribonucleic acid binding protein S1 Rnps1; ESTsMm 10622; contactin 3Mm 2968; Mus musculus glycoprotein 38; neurochondrinMm 43445; no match8; Mus musculus crystallin beta A4; S100 protein beta polypeptide neuralMm 829; Mm 37346; chromogranin BMm 1339; no match111; glial fibrillary acidic proteinMm 1239; Sugano mouse brain mncb MNCb 4842 5; Mus musculus Ly6 neurotoxin 1; ESTsMm 22801; Human Chromosome 7 clone RP11 297N5; proteolipid protein myelin Mm 1268; ESTs Weakly similar to F2 alpha prostoglandin regulatory protein M musculus Mm 29860; ESTsMm 28098; Mus musculus fibroblast growth factor 13; glutamate receptor ionotropic NMDA1 zeta 1 Mm 3292; amyloid beta A4 precursor protein binding family A member 2Mm 4657; ESTsMm 41808; Mus musculus zinc finger transcription factor Kaiso mRNA complete cdsMm 100832; R norvegicus mRNA for pro alpha 1 collagen type III; heat shock protein 25 kDa 2 cardiovascular Mm 103612; Mus musculus hypothetical protein 154; transcription factor 4Mm 4269; ESTs Highly similar to ATP SYNTHASE DELTA CHAIN MITOCHONDRIAL PRECURSOR Rattus norvegicus Mm 22514; M musculus vacuolar proton translocating ATPase 100 kDa subunit isoform a1 I; selenoprotein P plasma 1Mm 22699; solute carrier family 2 facilitated glucose transporter member 3Mm 3726; ESTsMm 33880; ESTsMm 34740; ESTsMm 29832; cathepsin DMm 2147; secretogranin IIIMm 2386; Mouse mRNA for neural cell adhesion molecu; Mus musculus glutathione S transferase mu 1; no match98; R norvegicus microtubule associated protein 1 A MAP1A; M musculus selenoprotein P; Mus musculus secreted phosphoprotein 1; ESTsMm 27363; ESTs Moderately similar to CALPONIN ACIDIC ISOFORM Rattus norvegicus Mm 22171; collapsin response mediator protein 1Mm 22695; insulin like growth factor binding protein 5Mm 578; ESTs Highly similar to neuroglycan C precursor R norvegicus Mm 38496; Mus musculus melastatin 1 Mlsn1; ceruloplasminMm 13787; ESTs Weakly similar to delta 6 fatty acid desaturase M musculus Mm 30158; ESTsMm 43499; Rattus norvegicus CD44 protein; M musculus G protein coupled receptor 37; UI M BH3 aun e 05 0 UI s1 NIH BMAP M S4; M musculus secreted acidic cysteine rich glycoprotein; nuclear receptor co repressor 1Mm 88061; ribosomal protein mitochondrial S7Mm 29902; Mm 104779; tropomodulin 2Mm 44216; M musculus insulin like growth factor binding protein 5; Mus musculus secreted acidic cysteine rich gly; Homo sapiens KIAA1077 protein; stearoyl Coenzyme A desaturase 2Mm 298; M musculus dickkopf homolog 3 Dkk 3; Mus musculus transketolase Tkt; L0283F 10 3 Mouse Newborn Ovary cDNA Library; Mus musculus neuron specific gene family member 2; prostaglandin D2 synthase 21 kDa brain Mm 1008; Mus neural cell adhesion molecule NCAM 140; Mouse brain specific small RNA; Mus musculus protein phosphatase 2; farnesyl diphosphate farnesyl transferase 1Mm 3204; M musculus proteolipid M6B isoform TMD psi M6B; Mouse brain neurofilament L; brain protein E46Mm 4098; Rattus norvegicus Spinophilin mRNA; ESTsMm 5258; ESTsMm 17436; Mouse heat shock protein hsp84; no match71; Mm 29846; R norvegicus n chimaerin; ESTsMm 10641; Mus musculus protein tyrosine phosphatase; Mm 100761; H sapiens transmembrane 4 superfamily member 7; H sapiens chromosome 3 clone RP11 19E8 map 3p; ESTsMm 26680; UI M BH3 avk f 09 0 UI s1 NIH BMAP M S4; ESTs Moderately similar to PRAJA 1 M musculus Mm 41711; Homo sapiens RNA binding protein BRUNOL4; actin beta cytoplasmicMm 103618; NCK associated protein 1Mm 25203; Mus musculus transcription factor 4 Tcf4; ESTsMm 39985; Mouse mRNA for OSF 1; ESTsMm 27030; Mouse cysteine rich glycoprotein; ESTsMm 71533; H sapiens membrane glycoprotein M6; Human hBOIT brain type organic ion transporter; dickkopf homolog 3 Xenopus laevis Mm 55143; no match23; DNA segment Chr 19 Wayne State University 55 expressedMm 29835; Mus musculus calpain 4; ESTs Highly similar to EXCITATORY AMINO ACID TRANSPORTER 1 M musculus Mm 33356; Ca2 dependent activator protein for secretionMm 5058; oxidative stress inducedMm 9846; adducin 1 alpha Mm 29052; ESTs Weakly similar to ORF YKR092c S cerevisiae Mm 24356; Mus musculus membrane protein TMS 2 mRNA complete cdsMm 29344; R norvegicus neurodegeneration associated protein 1; glutamine synthetaseMm 2338; ESTsMm 24254; Mus musculus clusterin; Mouse beta tubulin gene M beta 4 3 end; Mus musculus vimentin; Homo sapiens mRNA cDNA DKFZp586N1922; ESTsMm 27467; Mus musculus mRNA for profilin II pfn2 gene Mm 20399; protein L isoaspartate D aspartate O methyltransferase 1Mm 25293; ESTsMm 41819; ESTs Weakly similar to p190 B M musculus Mm 13835; anti oxidant protein 2Mm 6587; Mus musculus sulfated glycoprotein 2 isoform 2; ESTs Highly similar to zyginI R norvegicus Mm 5264; calmodulinMm 2648; and Homo sapiens membrane glycoprotein M6, with a test compound;
determining activity of said one or more NM proteins in said cells; and
identifying a test compound as a candidate drug for treating neuronal cell death if it decreases the activity of one more NM proteins in said cells.

43. The method of claim 42 wherein the cells are retinal cells.

44. The method of claim 42 wherein the cells are recombinant host cells which are transfected with an expression construct which encodes said one or more NMs.

45. A method to identify candidate drugs for treating neuronal cell death, comprising:
contacting cells which express one or more neuronal marker (NM) genes selected from the group consisting of Mus musculus retinal S antigen; Mus musculus neural retina leucine zipper gene; M musculus photoreceptor specific protein PSP G145; IMAGE 4507893 5; Mus musculus domesticus phosducin; IMAGE 4507284 5; Danio rerio brain type fatty acid binding protein; M musculus X linked juvenile retinoschisis protein; M musculus guanine nucleotide binding protein beta 1 Gnb1; Mus musculus TPA regulated locus; Mouse nuclear protein mdm 1; IMAGE 4511806 5; M musculus male germ cell associated kinase; heat shock protein 60 kDaMm 1777; no match17; NCI CGAP BC3 Mus musculus cDNA clone IMAGE 3976794; no homo16; Homo sapiens CGI 45 protein; ESTsMm 44103; Mouse opsin MOPS; IMAGE 4225062 5; Mm 100212; H sapiens fer fps fes related tyrosine kinase phosphoprotein NCP94 FER; IMAGE 4505626 5 602393946F1 NIH MGC 94; solute carrier family 12 member 2Mm 4168; Mus musculus BUB2 like protein 1 HBLP1 mRNA complete cdsMm 104771; hemoglobin Y beta like embryonic chainMm 35830; erythrocyte protein band 4 1Mm 30038; no match55; Mus musculus MYLE protein mRNA complete cdsMm 41091; RIKEN full length enriched adult male hypothalamus musculus cDNA clone A230050E13; NCI CGAP Mam6 Mus IMAGE 3500058; Mus musculus mRNA for GTP binding protein drg2 gene Mm 41803; Homo sapiens mRNA for KIAA1549 protein; Mus musculus karyopherin importin alpha 2 Kpna2; UI M BZ1 bkv b 01 0 UI 3; no match B; ESTsMm 939; Mus musculus cDNA sequence AF244542; IMAGE 1348390 5; solute carrier family 30 zinc transporter member 3Mm 1396; no match110; Mus musculus homeodomain protein crx; promininMm 6250; no homo13; IMAGE 1279184 5; Human microfibril associated glycoprotein 4; Mm 70462; no match A; Rattus sp mRNA for BHF 1; ribosomal protein S24Mm 16775; Stratagene mouse Tcell 937311 IMAGE 1002041; NCI CGAP Kid14 Mus IMAGE 4236354 5; R norvegicus retinoblastoma binding protein 9; Mus musculus exostoses multiple 1 Ext1; selectin endothelial cell ligandMm 488; ESTs Weakly similar to HYPOTHETICAL 16 1 KD PROTEIN IN SEC17 QCR1 INTERGENIC REGION Saccharomyces cerevisiae Mm 27114; ESTs Highly similar to KIAA0824 protein H sapiens Mm 34579; Mus musculus ribospsmal protein L10A Rp110a; R norvegicus ribonucleoprotein F; clone 1110007F23; no match38; M musculus Srp20 gene; homeodomain interacting protein kinase 2Mm 20934; FSHD region gene 1Mm 67; UI M BH3 ari c 10 0 UI s1 NIH BMAP M S4; Homo sapiens CED 6 protein CED 6; Mus musculus RIKEN clone 0610009E22; RAB18 member RAS oncogene familyMm 22660; no match5; Mus musculus prominin Prom; ribosomal protein L12Mm 70127; ESTs Highly similar to ELONGATION FACTOR 1 DELTA Homo sapiens Mm 21086; ESTs Highly similar to HYPOTHETICAL 37 2 KD PROTEIN C12C2 09C IN CHROMOSOME I Schizosaccharomyces pombe Mm 21383; clone 3021401C12; M musculus very long chain acyl CoA dehydrogenase; vitronectinMm 3667; ESTs Weakly similar to LIV 1 protein H sapiens Mm 41214; Mus musculus dopamine receptor 4; no match7; ATPase H transporting lysosomal vacuolar proton pump noncatalytic accessory protein 1 110 160.kDa Mm 20869; Rattus norvegicus partial mRNA for CRM1 protein; eukaryotic translation elongation factor 1 alpha 1Mm 16317; Human karyopherin beta2 importin; ESTs Moderately similar to hypothetical protein H sapiens Mm 22878; Homo sapiens PAC clone RP4 687K1; UI M AO1 aeh e 11 0 UI r1 NIH BMAP MPG N; high mobility group protein 14Mm 2756; ESTsMm 31374; R norvegicus aryl hydrocarbon interacting protein like 1; UI M CG0p bmu h 08 0 UI s1 NIH BMAP Ret4 S2; RAB10 member RAS oncogene familyMm 9455; Mus musculus early development regulator 2; no match83; Mus musculus topoisomerase DNA II beta; alpha tubulin; Homo sapiens MTA1 L1; retinitis pigmentosa GTPase regulator interacting protein 1 Mm 21662; Mus musculus FXYD dom containing ion transport regulator 5; Mus musculus cytochrome P450 3A25 CYP3A25 mRNA complete cdsMm 26993; IMAGE 4505626 5; RNA polymerase II transcriptional coactivatorMm 966; ESTs Highly similar to CAAX prenyl protease H sapiens Mm 34399; Soares mammary gland NbMMG IMAGE 1347586; clone 2700067D09; ESTs Weakly similar to defline not available 5901802 D melanogaster Mm 35127; torsin family 1 member AMm 29151; Mm 23086; M musculus brain cyclic nucleotide gated K; Mus musculus N myc downstream regulated 1; Homo sapiens splicing factor 3b subunit 3; Mus musculus mRNA for Lim homeodomain protein Islet1Mm 42242; Mouse mRNA for syntaxin 3D 1; Mus musculus chromosome 7 clone 19K5; ES18 proteinMm 23296; ESTs Highly similar to KIAA0729 protein H sapiens Mm 13148; ESTsMm 33949; Rat transcription factor RZR beta gene; ESTs Moderately similar to hypothetical protein H sapiens Mm 30235; Homo sapiens KIAA0009 gene product; no match X; ESTs Moderately similar to MYOSIN LIGHT CHAIN KINASE Dictyostelium discoideum Mm 1881; serum glucocorticoid regulated kinaseMm 28405; ESTs Weakly similar to cappuccino D melanogaster Mm 41762; regulator of G protein signaling 9Mm 38548; ESTsMm 34351; ESTsMm 32460; Mm 44404; ESTsMm 37515; Mus musculus cytochrome P450 2f2 Cyp2f2; Finkel Biskis Reilly murine sarcoma virus FBR MuSV ubiquitously expressed fox derived Mm 4890; guanylate cyclase activator 1 a retina Mm 16224; human CRX control; adducin 2 beta Mm 104155; mouse CRX control; NRL control; Mus musculus ELOVL4; Mus musculus N myc downstream regulated 3; lactate dehydrogenase 1 A chainMm 26504; ESTs Moderately similar to stromelysin PDGF responsive element binding protein transcription factor M musculus Mm 38372; ESTsMm 11285; M musculus chr 10 clone RP21 39C4; ESTs Highly similar to 40 KD PEPTIDYL PROLYL CIS TRANS ISOMERASE Homo sapiens Mm 30242; NIH BMAP Ret4 S2 Mus UI M CG0p big e 08 0 UI 3; Soares mammary gland NMLMG IMAGE 3467149; glycosylphosphatidylinositol 1 homolog human Mm 6354; Rattus norvegicus NMDA receptor subunit NR2; ESTsMm 33788; Mus musculus hexokinase 1 Hk1; inosine 5 phosphate dehydrogenase 2Mm 6065; N myc downstream regulated 3Mm 36775; no match V; villin 2Mm 4551; Rattus norvegicus TM6P1 TM6P1; Mus musculus mRNA for heterogeneous nuclear ribonucleoprotein HMm 21740; ESTsMm 103333; Mus musculus retinal taurine transporter; Mus musculus poly rC binding protein; ESTs Weakly similar to nuclear poly C binding protein M musculus Mm 29707; ESTs Weakly similar to similar to 1 acyl glycerol 3 phosphate acyltransferases C elegans Mm 24117; Mm 27013; pre B cell leukemia transcription factor 3Mm 7331; ESTsMm 21299; Mus musculus kinectin 1; Mus musculus drebrin A mRNA complete cdsMm 104044; H3087H01 5 NIA Mouse 15K cDNA Clone Set; SAC483 Mouse e14 5 developing pituitary gland; cloneE130113K08; Mus musculus major histocompatibility locus class II region Fas binding protein Daxx DAXX gene partial cds Bing1 BING1 tapasin tapasin RalGDS like factor RLF KE2 KE2 BING4 BING4 beta1 3 galactosyl transferase beta1 3 galactosylMm 20926; Mus musculus aquaporin 1; acyl Coenzyme A dehydrogenase very long chainMm 18630; Mouse proprotein convertase 4; M musculus activating transcription factor 4 Atf4; guanine nucleotide binding protein beta 5Mm 4702; phosducin control; ESTsMm 38578; Barstead bowel MPLRB9 IMAGE 1095982; M musculus stromal cell derived factor recep; ESTs Weakly similar to E04F6 2 gene product C elegans Mm 18889; IMAGE 963149 5; syntaxin binding protein 1 Mm 3129; solute carrier family 16 monocarboxylic acid transporters member 1Mm 9086; ESTs Highly similar to TRICARBOXYLATE TRANSPORT PROTEIN PRECURSOR Rattus norvegicus Mm 22679; Bcl2 likeMm 3882; Soares mouse p3NMF19 5 IMAGE 493296; Mus musculus beta galactosidase complex; H sapiens ADP ribosylation factor binding protein GGA2; Mm 31266; IMAGE 560050 5; Mus musculus DXHXS6673E protein DXHXS6673E mRNA complete cdsMm 23458; M musculus mRNA for hair keratin mHb6; Mus musculus thyroglobulin; ESTs Moderately similar to KIAA0956 protein H sapiens Mm 11428; H3050H05 3 NIA Mouse 15K cDNA Clone Set; ESTs Moderately similar to signal recognition particle 54K protein M musculus Mm 32508; Mouse PSD 95 SAP90A; ESTsMm 29308; alkaline phosphatase 2 liverMm 1265; Homo sapiens 12 seeders BAC RP11 19E18; ESTsMm 41269; ESTsMm 86724; Homo sapiens 12q13 1 PAC RPCI1 228P16; serine threonine kinase receptor associated proteinMm 22584; UI M BZ0 axl a 11 0 UI s1 NIH BMAP MHI2; Mus musculus poly rC binding protein 2; IMAGE 4503171 5; ESTsMm 35430; activating transcription factor 4Mm 641; Mouse serine threonine phosphatase 2C; GAPDH control; Human mRNA for KIAA0299; ESTs Weakly similar to proline rich protein M musculus Mm 41665; megakaryocyte associated tyrosine kinaseMm 2918; homer neuronal immediate early gene 2Mm 228; peroxisomal farnesylated proteinMm 29198; blank; zinc finger protein 238Mm 27962; ESTs Highly similar to PHENYLALANYL TRNA SYNTHETASE BETA CHAIN CYTOPLASMIC Saccharomyces cerevisiae Mm 27403; Rat microtubule associated protein 2 MAP2; timeless homolog Drosophila Mm 6458; kinectin 1Mm 3110; phosphatidylinositol membrane associatedMm 1860; R norvegicus CDP diacylglycerol synthase; Homo sapiens DKFZp434A132; Mus musculus hematopoietic zinc finger; mitogen activated protein kinase kinase 7Mm 3906; H3110G03 3 NIA Mouse 15K cDNA; ESTs Highly similar to HYPOTHETICAL 47 9 KD PROTEIN B0303 3 IN CHROMOSOME III Caenorhabditis elegans Mm 30147; ESTs Highly similar to CELL GROWTH REGULATING NUCLEOLAR PROTEIN M musculus Mm 28560; no match W; Mouse endogenous murine leukemia virus polytropic provirus DNA; clone1110013A05; aryl hydrocarbon receptorMm 4452; peroxisome proliferator activated receptor alphaMm 1373; Mus musculus LAG protein Lag Rattus NMDA receptor glutamate binding subunit; Mus musculus syntaxin binding protein 1; Mus musculus MAP kinase phosphatase 6; Rattus norvegicus retina specific protein PAL; no match33; Mus musculus myc box dependent interacting pro; Murine leukemia virus erv1 envelope protein; cytochrome c oxidase subunit Vila 3Mm 2151; proteasome prosome macropain subunit alpha type 3Mm 1007; Homo sapiens mRNA cDNA DKFZp434N1615; Mus musculus TCR beta locus; ESTs Weakly similar to LOK M musculus Mm 74661; small inducible cytokine subfamily A member 22Mm 12895; ESTsMm 23682; no match I; no match H; high mobility group protein I isoform CMm 3953; protein kinase cAMP dependent catalytic alphaMm 22479; Mus musculus phosphatidylinositol membrane associated; no match G; Mouse heparin binding epidermal growth factor like; Homo sapiens cDNA DKFZp586B0924; Mouse magnesium dependent protein; ESTs Weakly similar to ZW10 interactor Zwint H sapiens Mm 38994; ESTsMm 30480; H sapiens ADP ribosylation factor GTPase activating protein 1; Mus elongation of very long chain fatty acids; Mouse Y box binding protein 1 DNA binding MSY 1; Homo sapiens KIAA0249 gene product; Mus musculus Ran binding protein 2; Mus musculus histidine decarboxylase cluster; Homo sapiens cDNA FLJ21612 fis clone COL07355; UI M BH2 3 aqc g 10 0 UI 5; Rattus norvegicus APP binding protein 1; Mus musculus beta site APP cleaving enzyme; DNA methyltransferase cytosine 5 Mm 7814; no match66; ESTs Weakly similar to Lpi2p S cerevisiae Mm 21859; R norvegicus phosphatidylinositol synthase; ribonuclease L 2 5 oligoisoadenylate synthetase dependent inhibitorMm 5831; Mm 104074; H sapiens protein phosphatase 2A regulatory subunit B; H3147A11 5 NIA Mouse 15K cDNA Clone Set; Mus musculus Y box transcription factor; Mouse gene for basigin; Homo sapiens mRNA for FLJ00042 protein; R norvegicus nup155 nucleoporin 155kD; tubby like protein 1 Mm 42102; R norvegicus RNA binding protein SiahBP; UI M BZ0 axj h 06 0 UI 3; and Mus musculus pyruvate kinase 3, with a test compound;
determining expression of said one or more N\M genes by hybridization of mRNA of said cells to a nucleic acid probe which is complementary to said mRNA; and
identifying a test compound as a candidate drug for treating neuronal cell death if it increases expression of said one or more NM genes.

46. The method of claim 45 wherein the cells are retinal cells.

47. The method of claim 45 wherein the cells are recombinant host cells which are transfected with an expression construct which encodes said one or more NMs.

48. A method to identify candidate drugs for treating neuronal cell death, comprising:
contacting cells which express one or more neuronal marker (NM) proteins selected from the group consisting of: Mus musculus retinal S antigen; Mus musculus neural retina leucine zipper gene; M musculus photoreceptor specific protein PSP G145; IMAGE 4507893 5; Mus musculus domesticus phosducin; IMAGE 4507284 5; Danio rerio brain type fatty acid binding protein; M musculus X linked juvenile retinoschisis protein; M musculus guanine nucleotide binding protein beta 1 Gnb1; Mus musculus TPA regulated locus; Mouse nuclear protein mdm 1; IMAGE 4511806 5; M musculus male germ cell associated kinase; heat shock protein 60 kDaMm 1777; no match17; NCI CGAP BC3 Mus musculus cDNA clone IMAGE 3976794; no homo16; Homo sapiens CGI 45 protein; ESTsMm 44103; Mouse opsin MOPS; IMAGE 4225062 5; Mm 100212; H sapiens fer fps fes related tyrosine kinase phosphoprotein NCP94 FER; IMAGE 4505626 5 602393946F1 NIH MGC 94; solute carrier family 12 member 2Mm 4168; Mus musculus BUB2 like protein 1 HBLP1 mRNA complete cdsMm 104771; hemoglobin Y beta like embryonic chainMm 35830; erythrocyte protein band 4 1Mm 30038; no match55; Mus musculus MYLE protein mRNA complete cdsMm 41091; RIKEN full length enriched adult male hypothalamus musculus cDNA clone A230050E13; NCI CGAP Mam6 Mus IMAGE 3500058; Mus musculus mRNA for GTP binding protein drg2 gene Mm 41803; Homo sapiens mRNA for KIAA1549 protein; Mus musculus karyopherin importin alpha 2 Kpna2; UI M BZ1 bkv b 01 0 UI 3; no match B; ESTsMm 939; Mus musculus cDNA sequence AF244542; IMAGE 1348390 5; solute carrier family 30 zinc transporter member 3Mm 1396; no match110; Mus musculus homeodomain protein crx; promininMm 6250; no homo13; IMAGE 1279184 5; Human microfibril associated glycoprotein 4; Mm 70462; no match A; Rattus sp mRNA for BHF 1; ribosomal protein S24Mm 16775; Stratagene mouse Tcell 937311 IMAGE 1002041; NCI CGAP Kid14 Mus IMAGE 4236354 5; R norvegicus retinoblastoma binding protein 9; Mus musculus exostoses multiple 1 Ext1; selectin endothelial cell ligandMm 488; ESTs Weakly similar to HYPOTHETICAL 16 1 KD PROTEIN IN SEC17 QCR1 INTERGENIC REGION Saccharomyces cerevisiae Mm 27114; ESTs Highly similar to KIAA0824 protein H sapiens Mm 34579; Mus musculus ribospsmal protein L10A Rp110a; R norvegicus ribonucleoprotein F; clone 1110007F23; no match38; M musculus Srp20 gene; homeodomain interacting protein kinase 2Mm 20934; FSHD region gene 1Mm 67; UI M BH3 ari c 10 0 UI s1 NIH BMAP M S4; Homo sapiens CED 6 protein CED 6; Mus musculus RIKEN clone 0610009E22; RAB18 member RAS oncogene familyMm 22660; no match5; Mus musculus prominin Prom; ribosomal protein L12Mm 70127; ESTs Highly similar to ELONGATION FACTOR 1 DELTA Homo sapiens Mm 21086; ESTs Highly similar to HYPOTHETICAL 37 2 KD PROTEIN C12C2 09C IN CHROMOSOME I Schizosaccharomyces pombe Mm 21383; clone 3021401C12; M musculus very long chain acyl CoA dehydrogenase; vitronectinMm 3667; ESTs Weakly similar to LIV 1 protein H sapiens Mm 41214; Mus musculus dopamine receptor 4; no match7; ATPase H transporting lysosomal vacuolar proton pump noncatalytic accessory protein 1 110 160 kDa Mm 20869; Rattus norvegicus partial mRNA for CRM1 protein; eukaryotic translation elongation factor 1 alpha 1Mm 16317; Human karyopherin beta2 importin; ESTs Moderately similar to hypothetical protein H sapiens Mm 22878; Homo sapiens PAC clone RP4 687K1; UI M AO1 aeh e 11 0 UI r1 NIH BMAP MPG N; high mobility group protein 14Mm 2756; ESTsMm 31374; R norvegicus aryl hydrocarbon interacting protein like 1; UI M CG0p bmu h 08 0 UI s1 NIH BMAP Ret4 S2; RAB10 member RAS oncogene familyMm 9455; Mus musculus early development regulator 2; no match83; Mus musculus topoisomerase DNA II beta; alpha tubulin; Homo sapiens MTA1 L1; retinitis pigmentosa GTPase regulator interacting protein 1 Mm 21662; Mus musculus FXYD dom containing ion transport regulator 5; Mus musculus cytochrome P450 3A25 CYP3A25 mRNA complete cdsMm 26993; IMAGE 4505626 5; RNA polymerase II transcriptional coactivatorMm 966; ESTs Highly similar to CAAX prenyl protease H sapiens Mm 34399; Soares mammary gland NbMMG IMAGE 1347586; clone 2700067D09; ESTs Weakly similar to defline not available 5901802 D melanogaster Mm 35127; torsin family 1 member AMm 29151; Mm 23086; M musculus brain cyclic nucleotide gated K; Mus musculus N myc downstream regulated 1; Homo sapiens splicing factor 3b subunit 3; Mus musculus mRNA for Lim homeodomain protein Islet1Mm 42242; Mouse mRNA for syntaxin 3D 1; Mus musculus chromosome 7 clone 19K5; ES18 proteinMm 23296; ESTs Highly similar to KIAA0729 protein H sapiens Mm 13148; ESTsMm 33949; Rat transcription factor RZR beta gene; ESTs Moderately similar to hypothetical protein H sapiens Mm 30235; Homo sapiens KIAA0009 gene product; no match X; ESTs Moderately similar to MYOSIN LIGHT CHAIN KINASE Dictyostelium discoideum Mm 1881; serum glucocorticoid regulated kinaseMm 28405; ESTs Weakly similar to cappuccino D melanogaster Mm 41762; regulator of G protein signaling 9Mm 38548; ESTsMm 34351; ESTsMm 32460; Mm 44404; ESTsMm 37515; Mus musculus cytochrome P450 2f2 Cyp2f2; Finkel Biskis Reilly murine sarcoma virus FBR MuSV ubiquitously expressed fox derived Mm 4890; guanylate cyclase activator 1 a retina Mm 16224; human CRX control; adducin 2 beta Mm 104155; mouse CRX control; NRL control; Mus musculus ELOVL4; Mus musculus N myc downstream regulated 3; lactate dehydrogenase 1 A chainMm 26504; ESTs Moderately similar to stromelysin PDGF responsive element binding protein transcription factor M musculus Mm 38372; ESTsMm 11285; M musculus chr 10 clone RP21 39C4; ESTs Highly similar to 40 KD PEPTIDYL PROLYL CIS TRANS ISOMERASE Homo sapiens Mm 30242; NIH BMAP Ret4 S2 Mus UI M CG0p big e 08 0 UI 3; Soares mammary gland NMLMG IMAGE 3467149; glycosylphosphatidylinositol 1 homolog human Mm 6354; Rattus norvegicus NMDA receptor subunit NR2; ESTsMm 33788; Mus musculus hexokinase 1 Hk1; inosine 5 phosphate dehydrogenase 2Mm 6065; N myc downstream regulated 3Mm 36775; no match V; villin 2Mm 4551; Rattus norvegicus TM6P1 TM6P1; Mus musculus mRNA for heterogeneous nuclear ribonucleoprotein HMm 21740; ESTsMm 103333; Mus musculus retinal taurine transporter; Mus musculus poly rC binding protein; ESTs Weakly similar to nuclear poly C binding protein M musculus Mm 29707; ESTs Weakly similar to similar to 1 acyl glycerol 3 phosphate acyltransferases C elegans Mm 24117; Mm 27013; pre B cell leukemia transcription factor 3Mm 7331; ESTsMm 21299; Mus musculus kinectin 1; Mus musculus drebrin A mRNA complete cdsMm 104044; H3087H01 5 NIA Mouse 15K cDNA Clone Set; SAC483 Mouse e14 5 developing pituitary gland; cloneE130113K08; Mus musculus major histocompatibility locus class II region Fas binding protein Daxx DAXX gene partial cds Bing1 BING1 tapasin tapasin RalGDS like factor RLF KE2 KE2 BING4 BING4 beta1 3 galactosyl transferase beta1 3 galactosylMm 20926; Mus musculus aquaporin 1; acyl Coenzyme A dehydrogenase very long chainMm 18630; Mouse proprotein convertase 4; M musculus activating transcription factor 4 Atf4; guanine nucleotide binding protein beta 5Mm 4702; phosducin control; ESTsMm 38578; Barstead bowel MPLRB9 IMAGE 1095982; M musculus stromal cell derived factor recep; ESTs Weakly similar to E04F6 2 gene product C elegans Mm 18889; IMAGE 963149 5; syntaxin binding protein 1 Mm 3129; solute carrier family 16 monocarboxylic acid transporters member 1Mm 9086; ESTs Highly similar to TRICARBOXYLATE TRANSPORT PROTEIN PRECURSOR Rattus norvegicus Mm 22679; Bcl2 likeMm 3882; Soares mouse p3NMF19 5 IMAGE 493296; Mus musculus beta galactosidase complex; H sapiens ADP ribosylation factor binding protein GGA2; Mm 31266; IMAGE 560050 5; Mus musculus DXHXS6673E protein DXHXS6673E mRNA complete cdsMm 23458; M musculus mRNA for hair keratin mHb6; Mus musculus thyroglobulin; ESTs Moderately similar to KIAA0956 protein H sapiens Mm 11428; H3050H05 3 NIA Mouse 15K cDNA Clone Set; ESTs Moderately similar to signal recognition particle 54K protein M musculus Mm 32508; Mouse PSD 95 SAP90A; ESTsMm 29308; alkaline phosphatase 2 liverMm 1265; Homo sapiens 12 seeders BAC RP11 19E18; ESTsMm 41269; ESTsMm 86724; Homo sapiens 12q13 1 PAC RPCI1 228P16; serine threonine kinase receptor associated proteinMm 22584; UI M BZ0 axl a 11 0 UI s1 NIH BMAP MHI2; Mus musculus poly rC binding protein 2; IMAGE 4503171 5; ESTsMm 35430; activating transcription factor 4Mm 641; Mouse serine threonine phosphatase 2C; GAPDH control; Human mRNA for KIAA0299; ESTs Weakly similar to proline rich protein M musculus Mm 41665; megakaryocyte associated tyrosine kinaseMm 2918; homer neuronal immediate early gene 2Mm 228; peroxisomal farnesylated proteinMm 29198; blank; zinc finger protein 238Mm 27962; ESTs Highly similar to PHENYLALANYL TRNA SYNTHETASE BETA CHAIN CYTOPLASMIC Saccharomyces cerevisiae Mm 27403; Rat microtubule associated protein 2 MAP2; timeless homolog Drosophila Mm 6458; kinectin 1Mm 3110; phosphatidylinositol membrane associatedMm 1860; R norvegicus CDP diacylglycerol synthase; Homo sapiens DKFZp434A132; Mus musculus hematopoietic zinc finger; mitogen activated protein kinase kinase 7Mm 3906; H3110G03 3 NIA Mouse 15K cDNA; ESTs Highly similar to HYPOTHETICAL 47 9 KD PROTEIN B0303 3 IN CHROMOSOME III Caenorhabditis elegans Mm 30147; ESTs Highly similar to CELL GROWTH REGULATING NUCLEOLAR PROTEIN M musculus Mm 28560; no match W; Mouse endogenous murine leukemia virus polytropic provirus DNA; clone1110013A05; aryl hydrocarbon receptorMm 4452; peroxisome proliferator activated receptor alphaMm 1373; Mus musculus LAG protein Lag Rattus NMDA receptor glutamate binding subunit; Mus musculus syntaxin binding protein 1; Mus musculus MAP kinase phosphatase 6; Rattus norvegicus retina specific protein PAL; no match33; Mus musculus myc box dependent interacting pro; Murine leukemia virus erv1 envelope protein; cytochrome c oxidase subunit Vila 3Mm 2151; proteasome prosome macropain subunit alpha type 3Mm 1007; Homo sapiens mRNA cDNA DKFZp434N1615; Mus musculus TCR beta locus; ESTs Weakly similar to LOK M musculus Mm 74661; small inducible cytokine subfamily A member 22Mm 12895; ESTsMm 23682; no match I; no match H; high mobility group protein I isoform CMm 3953; protein kinase cAMP dependent catalytic alphaMm 22479; Mus musculus phosphatidylinositol membrane associated; no match G; Mouse heparin binding epidermal growth factor like; Homo sapiens cDNA DKFZp586B0924; Mouse magnesium dependent protein; ESTs Weakly similar to ZW10 interactor Zwint H sapiens Mm 38994; ESTsMm 30480; H sapiens ADP ribosylation factor GTPase activating protein 1; Mus elongation of very long chain fatty acids; Mouse Y box binding protein 1 DNA binding MSY 1; Homo sapiens KIAA0249 gene product; Mus musculus Ran binding protein 2; Mus musculus histidine decarboxylase cluster; Homo sapiens cDNA FLJ21612 fis clone COL07355; UI M BH2 3 aqc g 10 0 UI 5; Rattus norvegicus APP binding protein 1; Mus musculus beta site APP cleaving enzyme; DNA methyltransferase cytosine 5 Mm 7814; no match66; ESTs Weakly similar to Lpi2p S cerevisiae Mm 21859; R norvegicus phosphatidylinositol synthase; ribonuclease L 2 5 oligoisoadenylate synthetase dependent inhibitorMm 5831; Mm 104074; H sapiens protein phosphatase 2A regulatory subunit B; H3147A1 1 5 NIA Mouse 15K cDNA Clone Set; Mus musculus Y box transcription factor; Mouse gene for basigin; Homo sapiens mRNA for FLJ00042 protein; R norvegicus nup155 nucleoporin 155kD; tubby like protein 1 Mm 42102; R norvegicus RNA binding protein SiahBP; UI M BZ0 axj h 06 0 UI 3; and Mus musculus pyruvate kinase 3, with a test compound;
determining amount of said one or more NM proteins in said cells; and
identifying a test compound as a candidate drug for treating neuronal cell death if it increases the amount of one more NM proteins in said cells.

49. The method of claim 48 wherein the cells are retinal cells.

50. The method of claim 48 wherein the cells are recombinant host cells which are transfected with an expression construct which encodes said one or more NMs.

51. A method to identify candidate drugs for treating neuronal cell death, comprising:
contacting cells which express one or more neuronal marker (NM) proteins selected from the group consisting of: Mus musculus retinal S antigen; Mus musculus neural retina leucine zipper gene; M musculus photoreceptor specific protein PSP G145; IMAGE 4507893 5; Mus musculus domesticus phosducin; IMAGE 4507284 5; Danio rerio brain type fatty acid binding protein; M musculus X linked juvenile retinoschisis protein; M musculus guanine nucleotide binding protein beta 1 Gnb1; Mus musculus TPA regulated locus; Mouse nuclear protein mdm 1; IMAGE 4511806 5; M musculus male germ cell associated kinase; heat shock protein 60 kDaMm 1777; no match17; NCI CGAP BC3 Mus musculus cDNA clone IMAGE 3976794; no homo16; Homo sapiens CGI 45 protein; ESTsMm 44103; Mouse opsin MOPS; IMAGE 4225062 5; Mm 100212; H sapiens fer fps fes related tyrosine kinase phosphoprotein NCP94 FER; IMAGE 4505626 5 602393946F1 NIH MGC 94; solute carrier family 12 member 2Mm 4168; Mus musculus BUB2 like protein 1 HBLP1 mRNA complete cdsMm 104771; hemoglobin Y beta like embryonic chainMm 35830; erythrocyte protein band 4 1Mm 30038; no match55; Mus musculus MYLE protein mRNA complete cdsMm 41091; RIKEN full length enriched adult male hypothalamus musculus cDNA clone A230050E13; NCI CGAP Mam6 Mus IMAGE 3500058; Mus musculus mRNA for GTP binding protein drg2 gene Mm 41803; Homo sapiens mRNA for KIAA1549 protein; Mus musculus karyopherin importin alpha 2 Kpna2; UI M BZ1 bkv b 01 0 UI 3; no match B; ESTsMm 939; Mus musculus cDNA sequence AF244542; IMAGE 1348390 5; solute carrier family 30 zinc transporter member 3Mm 1396; no match110; Mus musculus homeodomain protein crx; promininMm 6250; no homo13; IMAGE 1279184 5; Human microfibril associated glycoprotein 4; Mm 70462; no match A; Rattus sp mRNA for BHF 1; ribosomal protein S24Mm 16775; Stratagene mouse Tcell 937311 IMAGE 1002041; NCI CGAP Kid14 Mus IMAGE 4236354 5; R norvegicus retinoblastoma binding protein 9; Mus musculus exostoses multiple 1 Ext1; selectin endothelial cell ligandMm 488; ESTs Weakly similar to HYPOTHETICAL 16 1 KD PROTEIN IN SEC17 QCR1 INTERGENIC REGION Saccharomyces cerevisiae Mm 27114; ESTs Highly similar to KLAA0824 protein H sapiens Mm 34579; Mus musculus ribospsmal protein L10A Rp110a; R norvegicus ribonucleoprotein F; clone 1110007F23; no match38; M musculus Srp20 gene; homeodomain interacting protein kinase 2Mm 20934; FSHD region gene 1Mm 67; UI M BH3 ari c 10 0 UI s1 NIH BMAP M S4; Homo sapiens CED 6 protein CED 6; Mus musculus RIKEN clone 0610009E22; RAB18 member RAS oncogene familyMm 22660; no match5; Mus musculus prominin Prom; ribosomal protein L12Mm 70127; ESTs Highly similar to ELONGATION FACTOR 1 DELTA Homo sapiens Mm 21086; ESTs Highly similar to HYPOTHETICAL 37 2 KD PROTEIN C12C2 09C IN CHROMOSOME I Schizosaccharomyces pombe Mm 21383; clone 3021401C12; M musculus very long chain acyl CoA dehydrogenase; vitronectinim 3667; ESTs Weakly similar to LIV 1 protein H sapiens Mm 41214; Mus musculus dopamine receptor 4; no match7; ATPase H transporting lysosomal vacuolar proton pump noncatalytic accessory protein 1 110 160 kDa Mm 20869; Rattus norvegicus partial mRNA for CRM1 protein; eukaryotic translation elongation factor 1 alpha 1 Mm 16317; Human karyopherin beta2 importin; ESTs Moderately similar to hypothetical protein H sapiens Mm 22878; Homo sapiens PAC clone RP4 687K1; UI M AO1 aeh e 11 0 UI r1 NIH BMAP MPG N; high mobility group protein 14Mm 2756; ESTsMm 31374; R norvegicus aryl hydrocarbon interacting protein like 1; UI M CG0p bmu h 08 0 UI s1 NIH BMAP Ret4 S2; RAB10 member RAS oncogene familyMm 9455; Mus musculus early development regulator 2; no match83; Mus musculus topoisomerase DNA II beta; alpha tubulin; Homo sapiens MTA1 L1; retinitis pigmentosa GTPase regulator interacting protein 1 Mm 21662; Mus musculus FXYD dom containing ion transport regulator 5; Mus musculus cytochrome P450 3A25 CYP3A25 mRNA complete cdsMm 26993; IMAGE 4505626 5; RNA polymerase II transcriptional coactivatorMm 966; ESTs Highly similar to CAAX prenyl protease H sapiens Mm 34399; Soares mammary gland NbMMG IMAGE 1347586; clone 2700067D09; ESTs Weakly similar to defline not available 5901802 D melanogaster Mm 35127; torsin family 1 member AMm 29151; Mm 23086; M musculus brain cyclic nucleotide gated K; Mus musculus N myc downstream regulated 1; Homo sapiens splicing factor 3b subunit 3; Mus musculus mRNA for Lim homeodomain protein Islet1Mm 42242; Mouse mRNA for syntaxin 3D 1; Mus musculus chromosome 7 clone 19K5; ES18 proteinMm 23296; ESTs Highly similar to KIAA0729 protein H sapiens Mm 13148; ESTsMm 33949; Rat transcription factor RZR beta gene; ESTs Moderately similar to hypothetical protein H sapiens Mm 30235; Homo sapiens KIAA0009 gene product; no match X; ESTs Moderately similar to MYOSIN LIGHT CHAIN KINASE Dictyostelium discoideum Mm 1881; serum glucocorticoid regulated kinaseMm 28405; ESTs Weakly similar to cappuccino D melanogaster Mm 41762; regulator of G protein signaling 9Mm 38548; ESTsMm 34351; ESTsMm 32460; Mm 44404; ESTsMm 37515; Mus musculus cytochrome P450 2f2 Cyp2f2; Finkel Biskis Reilly murine sarcoma virus FBR MuSV ubiquitously expressed fox derived Mm 4890; guanylate cyclase activator 1a retina Mm 16224; human CRX control; adducin 2 beta Mm 104155; mouse CRX control; NRL control; Mus musculus ELOVL4; Mus musculus N myc downstream regulated 3; lactate dehydrogenase 1 A chainMm 26504; ESTs Moderately similar to stromelysin PDGF responsive element binding protein transcription factor M musculus Mm 38372; ESTsMm 11285; M musculus chr 10 clone RP21 39C4; ESTs Highly similar to 40 KD PEPTIDYL PROLYL CIS TRANS ISOMERASE Homo sapiens Mm 30242; NIH BMAP Ret4 S2 Mus UI M CG0p big e 08 0 UI 3; Soares mammary gland NMLMG IMAGE 3467149; glycosylphosphatidylinositol 1 homolog human Mm 6354; Rattus norvegicus NMDA receptor subunit NR2; ESTsMm 33788; Mus musculus hexokinase 1 Hk1; inosine 5 phosphate dehydrogenase 2Mm 6065; N myc downstream regulated 3Mm 36775; no match V; villin 2Mm 4551; Rattus norvegicus TM6P 1 TM6P 1; Mus musculus mRNA for heterogeneous nuclear ribonucleoprotein HMm 21740; ESTsMm 103333; Mus musculus retinal taurine transporter; Mus musculus poly rC binding protein; ESTs Weakly similar to nuclear poly C binding protein M musculus Mm 29707; ESTs Weakly similar to similar to 1 acyl glycerol 3 phosphate acyltransferases C elegans Mm 24117; Mm 27013; pre B cell leukemia transcription factor 3Mm 7331; ESTsMm 21299; Mus musculus kinectin 1; Mus musculus drebrin A mRNA complete cdsMm 104044; H3087H01 5 NIA Mouse 15K cDNA Clone Set; SAC483 Mouse e14 5 developing pituitary gland; cloneE130113K08; Mus musculus major histocompatibility locus class II region Fas binding protein Daxx DAXX gene partial cds Bing1 BING1 tapasin tapasin RalGDS like factor RLF KE2 KE2 BING4 BING4 beta1 3 galactosyl transferase beta1 3 galactosylMm 20926; Mus musculus aquaporin 1; acyl Coenzyme A dehydrogenase very long chainMm 18630; Mouse proprotein convertase 4; M musculus activating transcription factor 4 Atf4; guanine nucleotide binding protein beta 5Mm 4702; phosducin control; ESTsMm 38578; Barstead bowel MPLRB9 IMAGE 1095982; M musculus stromal cell derived factor recep; ESTs Weakly similar to E04F6 2 gene product C elegans Mm 18889; IMAGE 963149 5; syntaxin binding protein 1 Mm 3129; solute carrier family 16 monocarboxylic acid transporters member 1Mm 9086; ESTs Highly similar to TRICARBOXYLATE TRANSPORT PROTEIN PRECURSOR Rattus norvegicus Mm 22679; Bcl2 likeMm 3882; Soares mouse p3NMF19 5 IMAGE 493296; Mus musculus beta galactosidase complex; H sapiens ADP ribosylation factor binding protein GGA2; Mm 31266; IMAGE 560050 5; Mus musculus DXHXS6673E protein DXHXS6673E mRNA complete cdsMm 23458; M musculus mRNA for hair keratin mHb6; Mus musculus thyroglobulin; ESTs Moderately similar to KIAA0956 protein H sapiens Mm 11428; H3050H05 3 NIA Mouse 15K cDNA Clone Set; ESTs Moderately similar to signal recognition particle 54K protein M musculus Mm 32508; Mouse PSD 95 SAP90A; ESTsMm 29308; alkaline phosphatase 2 liverMm 1265; Homo sapiens 12 seeders BAC RP11 19E18; ESTsMm 41269; ESTsMm 86724; Homo sapiens 12q13 1 PAC RPCI1 228P16; serine threonine kinase receptor associated proteinMm 22584; UI M BZ0 axl a 11 0 UI s1 NIH BMAP MHI2; Mus musculus poly rC binding protein 2; IMAGE 4503171 5; ESTsMm 35430; activating transcription factor 4Mm 641; Mouse serine threonine phosphatase 2C; GAPDH control; Human mRNA for KIAA0299; ESTs Weakly similar to proline rich protein M musculus Mm 41665; megakaryocyte associated tyrosine kinaseMm 2918; homer neuronal immediate early gene 2Mm 228; peroxisomal farnesylated proteinMm 29198; blank; zinc finger protein 238Mm 27962; ESTs Highly similar to PHENYLALANYL TRNA SYNTHETASE BETA CHAIN CYTOPLASMIC Saccharomyces cerevisiae Mm 27403; Rat microtubule associated protein 2 MAP2; timeless homolog Drosophila Mm 6458; kinectin 1Mm 3110; phosphatidylinositol membrane associatedMm 1860; R norvegicus CDP diacylglycerol synthase; Homo sapiens DKFZp434A132; Mus musculus hematopoietic zinc finger; mitogen activated protein kinase kinase 7Mm 3906; H3110G03 3 NIA Mouse 15K cDNA; ESTs Highly similar to HYPOTHETICAL 47 9 KD PROTEIN B0303 3 IN CHROMOSOME III Caenorhabditis elegans Mm 30147; ESTs Highly similar to CELL GROWTH REGULATING NUCLEOLAR PROTEIN M musculus Mm 28560; no match W; Mouse endogenous murine leukemia virus polytropic provirus DNA; clone1110013A05; aryl hydrocarbon receptorMm 4452; peroxisome proliferator activated receptor alphaMm 1373; Mus musculus LAG protein Lag Rattus NMDA receptor glutamate binding subunit; Mus musculus syntaxin binding protein 1; Mus musculus MAP kinase phosphatase 6; Rattus norvegicus retina specific protein PAL; no match33; Mus musculus myc box dependent interacting pro; Murine leukemia virus erv1 envelope protein; cytochrome c oxidase subunit VIIa 3Mm 2151; proteasome prosome macropain subunit alpha type 3Mm 1007; Homo sapiens mRNA cDNA DKFZp434N1615; Mus musculus TCR beta locus; ESTs Weakly similar to LOK M musculus Mm 74661; small inducible cytokine subfamily A member 22Mm 12895; ESTsMm 23682; no match I; no match H; high mobility group protein I isoform CMm 3953; protein kinase cAMP dependent catalytic alphaMm 22479; Mus musculus phosphatidylinositol membrane associated; no match G; Mouse heparin binding epidermal growth factor like; Homo sapiens cDNA DKFZp586B0924; Mouse magnesium dependent protein; ESTs Weakly similar to ZW10 interactor Zwint H sapiens Mm 38994; ESTsMm 30480; H sapiens ADP ribosylation factor GTPase activating protein 1; Mus elongation of very long chain fatty acids; Mouse Y box binding protein 1 DNA binding MSY 1; Homo sapiens KIAA0249 gene product; Mus musculus Ran binding protein 2; Mus musculus histidine decarboxylase cluster; Homo sapiens cDNA FLJ21612 fis clone COL07355; UI M BH2 3 aqc g 10 0 UI 5; Rattus norvegicus APP binding protein 1; Mus musculus beta site APP cleaving enzyme; DNA methyltransferase cytosine 5 Mm 7814; no match66; ESTs Weakly similar to Lpi2p S cerevisiae Mm 21859; R norvegicus phosphatidylinositol synthase; ribonuclease L 2 5 oligoisoadenylate synthetase dependent inhibitorMm 5831; Mm 104074; H sapiens protein phosphatase 2A regulatory subunit B; H3147A11 5 NIA Mouse 15K cDNA Clone Set; Mus musculus Y box transcription factor; Mouse gene for basigin; Homo sapiens mRNA for FLJ00042 protein; R norvegicus nup155 nucleoporin 155kD; tubby like protein 1 Mm 42102; R norvegicus RNA binding protein SiahBP; UI M BZ0 axj h 06 0 UI 3; and Mus musculus pyruvate kinase 3, with a test compound;
determining activity of said one or more NM proteins in said cells; and
identifying a test compound as a candidate drug for treating neuronal cell death if it increases the activity of one more NM proteins in said cells.

52. The method of claim 51 wherein the cells are retinal cells.

53. The method of claim 51 wherein the cells are recombinant host cells which are transfected with an expression construct which encodes said one or more NMs.


SEQUENCE LISTING (16 sequences)

SEQ ID NO: 1 - DNA, 18 bp, Mus musculus
atctgctgcatctgcttg

SEQ ID NO: 2 - DNA, 20 bp, Mus musculus
cgacctggaagtccaactac

SEQ ID NO: 3 - DNA, 19 bp, Mus musculus
attacgaccacgacagctc

SEQ ID NO: 4 - DNA, 19 bp, Mus musculus
ttcgtcacgtttgtcactg

SEQ ID NO: 5 - DNA, 18 bp, Mus musculus
agctgacaggcacaaagc

SEQ ID NO: 6 - DNA, 18 bp, Mus musculus
cagtaaagaatcccgaga

SEQ ID NO: 7 - DNA, 19 bp, Mus musculus
gaaactgcacgatgaagag

SEQ ID NO: 8 - DNA, 20 bp, Mus musculus
taggtggcgatctcaatgtc

SEQ ID NO: 9 - DNA, 19 bp, Mus musculus
atggcaggagcatgaaatg

SEQ ID NO: 10 - DNA, 20 bp, Mus musculus
tagtagtggtgatcatggtg

SEQ ID NO: 11 - DNA, 21 bp, Mus musculus
agacagtcattacatctgtag

SEQ ID NO: 12 - DNA, 19 bp, Mus musculus
gtgatgagatccagcagct

SEQ ID NO: 13 - DNA, 19 bp, Mus musculus
aatgacaggtggcttgaac

SEQ ID NO: 14 - DNA, 20 bp, Mus musculus
gtgtataagcgatactacga

SEQ ID NO: 15 - DNA, 19 bp, Mus musculus
cagggttcaggtttggttc

SEQ ID NO: 16 - DNA, 19 bp, Mus musculus
catctgtggagggtcttgg